SEQUENCE LISTING (4 sequences)

SEQ ID NO: 1 - DNA, 25 bp, synthetic construct
  source 1..25: mol_type = other DNA
caccgcaggccgcggccgatgagat

SEQ ID NO: 2 - DNA, 25 bp, synthetic construct
  source 1..25: mol_type = other DNA
aaacatctcatcggccgcggcctgc

SEQ ID NO: 3 - DNA, 25 bp, synthetic construct
  source 1..25: mol_type = other DNA
caccgacaggtgttcccgttgtccc

SEQ ID NO: 4 - DNA, 25 bp, synthetic construct
  source 1..25: mol_type = other DNA
aaacgggacaacgggaacacctgtc

(19) United States
(12) Patent Application Publication
(10) Pub. No.: US 2026/0014129 A1
(43) Pub. Date: Jan. 15, 2026

(54) MITOCHONDRIAL PYRUVATE METABOLISM INHIBITORS FOR TREATING CHRONIC MYELOID LEUKEMIA

(71) Applicant: The University Court of the University of Glasgow, Strathclyde (GB)

(72) Inventors: Gudmundur Vignir HELGASON, Strathclyde (GB); Kevin Michael RATTIGAN, Strathclyde (GB)

(73) Assignee: The University Court of the University of Glasgow, Strathclyde, GB (GB)

(21) Appl. No.: 18/995,488

(22) PCT Filed: Jul. 17, 2023
(86) PCT No.: PCT/EP2023/069839

§ 371 Date: Jan. 16, 2025

(30) Foreign Application Priority Data
Jul. 18, 2022 (GB) 2210503.5

(51) Int. Cl. A61K 31/4439; A61K 31/426; A61K 31/496; A61K 31/5025; A61K 31/506; A61P 35/02

(52) U.S. Cl. CPC A61K 31/4439; A61K 31/426; A61K 31/496; A61K 31/5025; A61K 31/506; A61P 35/02

(57) ABSTRACT

The invention relates to the treatment of chronic myeloid leukemia (CML). In particular it relates to the treatment of CML with inhibitors of mitochondrial pyruvate transport, which are able to target leukemic stem cells (LSCs) which are resistant to therapy with tyrosine kinase inhibitors (TKIs). Combination therapies with BCR-ABL kinase inhibitors are also described.

This application claims priority from GB 2210503.5, filed 18 Jul. 2022, the contents and elements of which are herein incorporated by reference for all purposes.

### FIELD OF THE INVENTION

The invention relates to the treatment of chronic myeloid leukemia (CML). In particular it relates to the finding that inhibitors of mitochondrial pyruvate transport may help to target leukemic stem cells (LSCs) which are resistant to therapy with tyrosine kinase inhibitors (TKIs).

### BACKGROUND

Deregulation of oxidative metabolism has been reported in multiple types of leukaemia (1-3). Other deregulated metabolic pathways include lipid metabolism, branch chained amino acid metabolism and glutamine metabolism (4-7). Several studies have examined how metabolic pathways may contribute to resistance to standard therapeutics such as cytarabine or venetoclax, and found it is driven by pathways such as oxidative metabolism, lipid metabolism, nicotinamide metabolism or cellular reduction-oxidation (redox) alterations (5, 8-11). While the tricarboxylic acid (TCA) cycle generates NADH and FADH₂ that are used to generate ATP through oxidative phosphorylation within the electron transport chain, intermediates of the cycle are also used for anabolic purposes such as nucleotide synthesis (via aspartate) or fatty acid synthesis (via citrate), necessitating the replenishment of intermediates through input of additional carbons.

Anaplerosis is a metabolic process that refills the TCA cycle with carbons to compensate for those extracted from the cycle (cataplerosis) for biosynthetic processes such as nucleotide or fatty acid synthesis. While fatty acid oxidation only supplies 2 carbons to the TCA cycle, glutamine can contribute 4 or 5 carbons (via glutamine oxidation or reductive carboxylation, respectively). During glycolysis, six-carbon glucose is converted to two three-carbon pyruvate molecules. Glucose-derived pyruvate provides 2 carbons to the TCA cycle when pyruvate is metabolised by pyruvate dehydrogenase (PDH; catalysis pyruvate to acetyl CoA reaction), but adds 3 carbons via pyruvate carboxylase (PC; catalysis carboxylation of pyruvate to form oxaloacetate), with both reactions upregulated in primary leukaemia cells (12).

Chronic myeloid leukaemia (CML) is a myeloproliferative disorder that is caused by a reciprocal translocation between chromosomes 9 and 22 t(9;22)(q34;q11) that leads to the formation of the Philadelphia chromosome (13, 14). This translocation generates a constitutively active BCR-ABL oncogenic fusion protein (15). Most CML patients present in chronic phase before inexorably progressing to the more aggressive accelerated phase or lethal blast phase if left untreated (16). Due to the lack of genetic complexity when compared with other types of leukaemia, and its well-defined leukaemic stem cell (LSC) population, chronic phase CML is a paradigm for targeted therapy and thus a model disease to examine how LSCs respond to anti-cancer treatments.

While the use of tyrosine kinase inhibitors (TKIs) such as imatinib (Gleevec) have significantly prolonged patient survival (Druker et al., 2006), the failure of TKIs to eradicate disease-initiating LSCs means that treatment discontinuation is unsuccessful for the majority of patients (17, 18). Additionally, as continual treatment enables more patients to survive longer without progressing to the fatal blast phase, the prevalence of the disease is increasing and CML is expected to become the most common leukaemia type within 30 years (19). Several explanations for the TKI resistance of LSCs have been reported, including BCR-ABL kinase domain mutations, higher levels of BCR-ABL protein, BCR-ABL independent pathways and contribution of the bone marrow niche (20-24).

Therapies which target the LSC population would provide a number of advantages, including an increased chance of progressing to therapy-free remission, and reduced risk of developing resistance to TKIs.

### SUMMARY OF THE INVENTION

The inventors have found that agents which inhibit mitochondrial pyruvate metabolism, such as inhibitors of pyruvate transport into mitochondria, are capable of targeting the leukemic stem cell (LSC) population in chronic myeloid leukemia (CML). In particular, they are capable of sensitising the LSC population to tyrosine kinase inhibitors (TKIs), as well as directly inhibiting expansion of (e.g. reducing) the LSC population.

Pyruvate transport into mitochondria is mediated by the mitochondrial pyruvate carrier (MPC). Thus an agent which inhibits mitochondrial pyruvate transport may be designated as an MPC inhibitor.

The invention provides an MPC inhibitor for use in the treatment of chronic myeloid leukemia (CML), wherein the MPC inhibitor is a compound of Formula (I) or a pharmaceutically acceptable salt thereof:

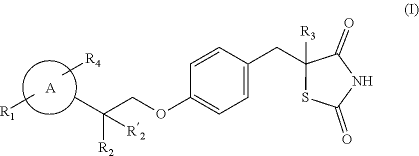

wherein
   each of R₁ and R₄ is independently selected from —H, halo, C₁₋₆ alkyl, and C₁₋₆ alkoxy, wherein the C₁₋₆ alkyl and C₁₋₆ alkoxy are optionally substituted with 1-3 of halo;
   R′₂ is —H and R₂ is selected from halo, hydroxy, optionally substituted C₁₋₆ alkyl, —OC(O)R^{A}, —O(SO₂)NH₂, —OCH(Rₘ)OC(O)Rₙ, —OCH(Rₘ)OP(O)(ORₙ)₂, —OP(O)(ORₙ)₂, and

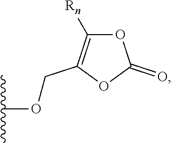

Wherein R^{A} is selected from optionally substituted C₁₋₁₂ alkyl, optionally substituted C₁₋₁₂ aryl and optionally substituted C₅₋₁₂ heteroaryl, each Rₘ is independently optionally substituted C₁₋₁₂ alkyl, each Rₙ is independently selected from optionally substituted C₁₋₁₂ alkyl, optionally substituted C₃₋₈ cycloalkyl, and optionally substituted phenyl; or R₂ and R′₂ together form oxo;
R₃ is —H or optionally substituted C₁₋₃ alkyl; and
A is a phenyl, pyridin-2-yl, pyridin-3-yl, or pyridin-4-yl.

The use may reside in the specific targeting of the LSC population in CML, e.g. in order to directly inhibit expansion of the LSC population, and/or to sensitise LSCs to TKI treatment.

The term “inhibiting expansion” is used to encompass slowing of the rate of increase of the stem cell population as compared to the rate of increase in the absence of treatment, and to holding the population size steady, but also actively reducing the size of the stem cell population. Without wishing to be bound by theory, the MPC inhibitor may exert its effect on the LSC population by inhibiting LSC proliferation, inducing LSC differentiation, and/or inducing LSC death (e.g. by apoptosis), independently of its sensitising effect to TKIs.

Typically, the subject to be treated will also be receiving treatment with a TKI which is an inhibitor of the BCR-ABL kinase. Thus the MPC inhibitor may be for administration in conjunction with a BCR-ABL kinase inhibitor.

Thus the invention provides an MPC inhibitor for use in
(i) inhibiting expansion of the LSC population in CML; and/or
(ii) sensitising LSCs to treatment with a BCR-ABL kinase inhibitor in CML;
wherein the MPC inhibitor is a compound of Formula (I) or a pharmaceutically acceptable salt thereof:

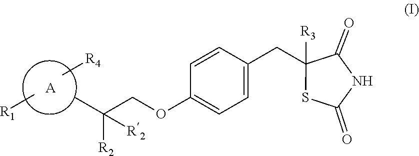

wherein
   each of R₁ and R₄ is independently selected from —H, halo, C₁₋₆ alkyl, and C₁₋₆ alkoxy, wherein the C₁₋₆ alkyl and C₁₋₆ alkoxy are optionally substituted with 1-3 of halo;
   R′₂ is —H and R₂ is selected from halo, hydroxy, optionally substituted C₁₋₆ alkyl, —OC(O)R^{A}, —O(SO₂)NH₂, —OCH(Rₘ)OC(O)Rₙ, —OCH(Rₘ)OP(O)(ORₙ)₂, —OP(O)(ORₙ)₂, and

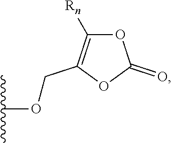

wherein R^{A} is selected from optionally substituted C₁₋₁₂ alkyl, optionally substituted C₆₋₁₂ aryl and optionally substituted C₅₋₁₂ heteroaryl, each Rₘ is independently optionally substituted C₁₋₁₂ alkyl, each Rₙ is independently selected from optionally substituted C₁₋₁₂ alkyl, optionally substituted C₃₋₈ cycloalkyl, and optionally substituted phenyl; or R₂ and R′₂ together form oxo;
R₃ is —H or optionally substituted C₁₋₃ alkyl; and
A is a phenyl, pyridin-2-yl, pyridin-3-yl, or pyridin-4-yl.

The invention further provides a combination therapy comprising
(a) an MPC inhibitor; and
(b) a BCR-ABL kinase inhibitor;
for treatment of CML;
wherein the MPC inhibitor is a compound of Formula (I) or a pharmaceutically acceptable salt thereof:

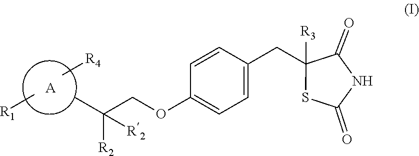

wherein
   each of R₁ and R₄ is independently selected from —H, halo, C₁₋₆ alkyl, and C₁₋₆ alkoxy, wherein the C₁₋₆ alkyl and C₁₋₆ alkoxy are optionally substituted with 1-3 of halo;
   R′₂ is —H and R₂ is selected from halo, hydroxy, optionally substituted C₁₋₆ alkyl, —OC(O)R^{A}, —O(SO₂)NH₂, —OCH(Rₘ)OC(O)Rₙ, —OCH(Rₘ)OP(O)(ORₙ)₂, —OP(O)(ORₙ)₂, and

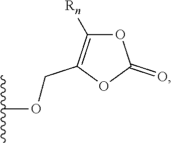

wherein R^{A} is selected from optionally substituted C₁₋₁₂ alkyl, optionally substituted C₆₋₁₂ aryl and optionally substituted C₅₋₁₂ heteroaryl, each Rₘ is independently optionally substituted C₁₋₁₂ alkyl, each Rₙ is independently selected from optionally substituted C₁₋₁₂ alkyl, optionally substituted C₃₋₈ cycloalkyl, and optionally substituted phenyl; or R₂ and R′₂ together form oxo;
R₃ is —H or optionally substituted C₁₋₃ alkyl; and
A is a phenyl, pyridin-2-yl, pyridin-3-yl, or pyridin-4-yl.

In the combination therapy, the MPC inhibitor may be administered for
(i) inhibiting expansion of the LSC population; and/or
(ii) sensitising LSCs to treatment with a BCR-ABL kinase inhibitor.

The invention further provides an MPC inhibitor for use in the treatment of chronic myeloid leukemia (CML), wherein the MPC inhibitor is for administration in combination with a BCR-ABL kinase inhibitor, and wherein the MPC inhibitor is a compound of Formula (I) or a pharmaceutically acceptable salt thereof:

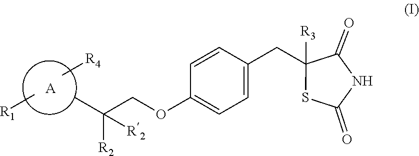

wherein
   each of R₁ and R₄ is independently selected from —H, halo, C₁₋₆ alkyl, and C₁₋₆ alkoxy, wherein the C₁₋₆ alkyl and C₁₋₆ alkoxy are optionally substituted with 1-3 of halo;
   R′₂ is —H and R₂ is selected from halo, hydroxy, optionally substituted C₁₋₆ alkyl, —OC(O)R^{A}, —O(SO₂)NH₂, —OCH(Rₘ)OC(O)Rₙ, —OCH(Rₘ)OP(O)(ORₙ)₂, —OP(O)(ORₙ)₂, and

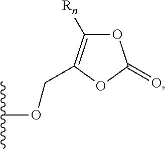

wherein R^{A} is selected from optionally substituted C₁₋₁₂ alkyl, optionally substituted C₆₋₁₂ aryl and optionally substituted C₅₋₁₂ heteroaryl, each Rₘ is independently optionally substituted C₁₋₁₂ alkyl, each Rₙ is independently selected from optionally substituted C₁₋₁₂ alkyl, optionally substituted C₃₋₈ cycloalkyl, and optionally substituted phenyl; or R₂ and R′₂ together form oxo;
R₃ is —H or optionally substituted C₁₋₃ alkyl; and
A is a phenyl, pyridin-2-yl, pyridin-3-yl, or pyridin-4-yl.

The invention further provides an MPC inhibitor, for use in
(i) inhibiting expansion of the LSC population in CML; and/or
(ii) sensitising LSCs to treatment with a BCR-ABL kinase inhibitor in CML;
wherein the MPC inhibitor is for administration in combination with a BCR-ABL kinase inhibitor, and
wherein the MPC inhibitor is a compound of Formula (I) or a pharmaceutically acceptable salt thereof:

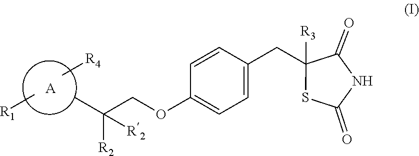

wherein
   each of R₁ and R₄ is independently selected from —H, halo, C₁₋₆ alkyl, and C₁₋₆ alkoxy, wherein the C₁₋₆ alkyl and C₁₋₆ alkoxy are optionally substituted with 1-3 of halo;
   R′₂ is —H and R₂ is selected from halo, hydroxy, optionally substituted C₁₋₆ alkyl, —OC(O)R^{A}, —O(SO₂)NH₂, —OCH(Rₘ)OC(O)Rₙ, —OCH(Rₘ)OP(O)(ORₙ)₂, —OP(O)(ORₙ)₂, and

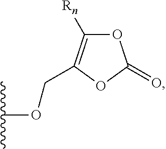

wherein R^{A} is selected from optionally substituted C₁₋₁₂ alkyl, optionally substituted C₆₋₁₂ aryl and optionally substituted C₅₋₁₂ heteroaryl, each Rₘ is independently optionally substituted C₁₋₁₂ alkyl, each Rₙ is independently selected from optionally substituted C₁₋₁₂ alkyl, optionally substituted C₃₋₈ cycloalkyl, and optionally substituted phenyl; or R₂ and R′₂ together form oxo;
R₃ is —H or optionally substituted C₁₋₃ alkyl; and
A is a phenyl, pyridin-2-yl, pyridin-3-yl, or pyridin-4-yl.

The invention further provides a BCR-ABL kinase inhibitor for use in the treatment of CML, wherein the BCR-ABL kinase inhibitor is for administration in combination with an MPC inhibitor of Formula (I) or a pharmaceutically acceptable salt thereof:

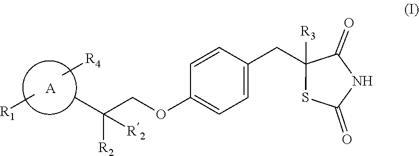

wherein
   each of R₁ and R₄ is independently selected from —H, halo, C₁₋₆ alkyl, and C₁₋₆ alkoxy, wherein the C₁₋₆ alkyl and C₁₋₆ alkoxy are optionally substituted with 1-3 of halo;
   R′₂ is —H and R₂ is selected from halo, hydroxy, optionally substituted C₁₋₆ alkyl, —OC(O)R^{A}, —O(SO₂)NH₂, —OCH(Rₘ)OC(O)Rₙ, —OCH(Rₘ)OP(O)(ORₙ)₂, —OP(O)(ORₙ)₂, and

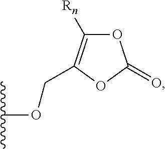

wherein R^{A} is selected from optionally substituted C₁₋₁₂ alkyl, optionally substituted C₆₋₁₂ aryl and optionally substituted C₅₋₁₂ heteroaryl, each Rₘ is independently optionally substituted C₁₋₁₂ alkyl, each Rₙ is independently selected from optionally substituted C₁₋₁₂ alkyl, optionally substituted C₃₋₈ cycloalkyl, and optionally substituted phenyl; or R₂ and R′₂ together form oxo;
R₃ is —H or optionally substituted C₁₋₃ alkyl; and
A is a phenyl, pyridin-2-yl, pyridin-3-yl, or pyridin-4-yl.

The MPC inhibitor may be administered for
(i) inhibiting LSC proliferation; and/or
(ii) sensitising LSCs to treatment with a BCR-ABL kinase inhibitor.

In all aspects in which two active agents are administered (i.e. an MPC inhibitor and a BCR-ABL inhibitor), they may be administered to a subject simultaneously or substantially simultaneously, e.g. within 1 hour of each other. Alternatively they may be administered at different times, e.g. spaced by at least 1 hour, at least 6 hours, at least 12 hours, or at least 24 hours. Still further alternatively, they may be administered according to different administration regimes.

Where they are for administration together, they may be formulated together in the same composition, or in separate compositions. Desirably, they may each be formulated for oral administration, whether together or separately.

The invention further provides a method of treating CML in a subject, comprising administering an MPC inhibitor to the subject, wherein the MPC inhibitor is a compound of Formula (I) or a pharmaceutically acceptable salt thereof:

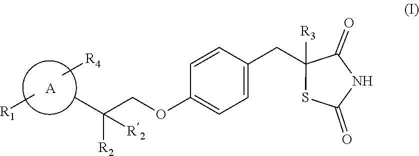

wherein
   each of R₁ and R₄ is independently selected from —H, halo, C₁₋₆ alkyl, and C₁₋₆ alkoxy, wherein the C₁₋₆ alkyl and C₁₋₆ alkoxy are optionally substituted with 1-3 of halo;
   R′₂ is —H and R₂ is selected from halo, hydroxy, optionally substituted C₁₋₆ alkyl, —OC(O)R^{A}, —O(SO₂)NH₂, —OCH(Rₘ)OC(O)Rₙ, —OCH(Rₘ)OP(O)(ORₙ)₂, —OP(O)(ORₙ)₂, and

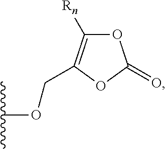

wherein R^{A} is selected from optionally substituted C₁₋₁₂ alkyl, optionally substituted C₆₋₁₂ aryl and optionally substituted C₅₋₁₂ heteroaryl, each Rₘ is independently optionally substituted C₁₋₁₂ alkyl, each Rₙ is independently selected from optionally substituted C₁₋₁₂ alkyl, optionally substituted C₃₋₈ cycloalkyl, and optionally substituted phenyl; or R₂ and R′₂ together form oxo;
R₃ is —H or optionally substituted C₁₋₃ alkyl; and
A is a phenyl, pyridin-2-yl, pyridin-3-yl, or pyridin-4-yl an MPC

The subject may also be receiving treatment with a BCR-ABL kinase inhibitor. Thus the method may further comprise administering a BCR-ABL kinas inhibitor to the subject.

The invention further provides a method of:
(i) inhibiting expansion of the LSC population in a subject with CML; and/or
(ii) sensitising LSCs to treatment with a BCR-ABL kinase inhibitor in a subject with CML;
comprising administering an MPC inhibitor to the subject;
wherein the MPC inhibitor is a compound of Formula (I) or a pharmaceutically acceptable salt thereof:

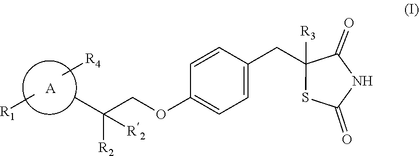

wherein
   each of R₁ and R₄ is independently selected from —H, halo, C₁₋₆ alkyl, and C₁₋₆ alkoxy, wherein the C₁₋₆ alkyl and C₁₋₆ alkoxy are optionally substituted with 1-3 of halo;
   R′₂ is —H and R₂ is selected from halo, hydroxy, optionally substituted C₁₋₆ alkyl, —OC(O)R^{A}, —O(SO₂)NH₂, —OCH(Rₘ)OC(O)Rₙ, —OCH(Rₘ)OP(O)(ORₙ)₂, —OP(O)(ORₙ)₂, and

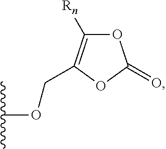

wherein R^{A} is selected from optionally substituted C₁₋₁₂ alkyl, optionally substituted C₆₋₁₂ aryl and optionally substituted C₅₋₁₂ heteroaryl, each Rₘ is independently optionally substituted C₁₋₁₂ alkyl, each Rₙ is independently selected from optionally substituted C₁₋₁₂ alkyl, optionally substituted C₃₋₈ cycloalkyl, and optionally substituted phenyl; or R₂ and R′₂ together form oxo;
R₃ is —H or optionally substituted C₁₋₃ alkyl; and
A is a phenyl, pyridin-2-yl, pyridin-3-yl, or pyridin-4-yl.

The invention further provides the use of an MPC inhibitor in the manufacture of a medicament for use in the treatment of CML, wherein the MPC inhibitor is a compound of Formula (I) or a pharmaceutically acceptable salt thereof:

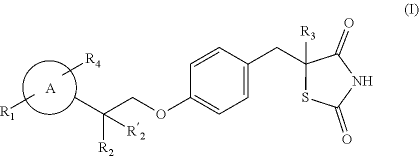

wherein
   each of R₁ and R₄ is independently selected from —H, halo, C₁₋₆ alkyl, and C₁₋₆ alkoxy, wherein the C₁₋₆ alkyl and C₁₋₆ alkoxy are optionally substituted with 1-3 of halo;
   R′₂ is —H and R₂ is selected from halo, hydroxy, optionally substituted C₁₋₆ alkyl, —OC(O)R^{A}, —O(SO₂)NH₂, —OCH(Rₘ)OC(O)Rₙ, —OCH(Rₘ)OP(O)(ORₙ)₂, —OP(O)(ORₙ)₂, and

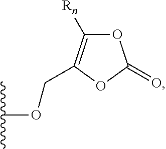

wherein R^{A} is selected from optionally substituted C₁₋₁₂ alkyl, optionally substituted C₆₋₁₂ aryl and optionally substituted C₅₋₁₂ heteroaryl, each Rₘ is independently optionally substituted C₁₋₁₂ alkyl, each Rₙ is independently selected from optionally substituted C₁₋₁₂ alkyl, optionally substituted C₃₋₈ cycloalkyl, and optionally substituted phenyl; or R₂ and R′₂ together form oxo;
R₃ is —H or optionally substituted C₁₋₃ alkyl; and
A is a phenyl, pyridin-2-yl, pyridin-3-yl, or pyridin-4-yl.

The subject to be treated may also be receiving treatment with a BCR-ABL kinase inhibitor. Thus the medicament may be for use in conjunction with a BCR-ABL kinase inhibitor.

The invention further provides the use of
(i) an MPC inhibitor; and
(ii) a BCR-ABL kinase inhibitor;
in the manufacture of a medicament for use in the treatment of CML;
wherein the MPC inhibitor is a compound of Formula (I) or a pharmaceutically acceptable salt thereof:

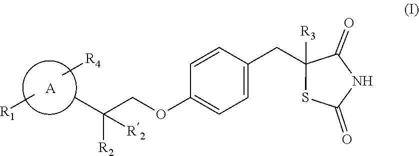

wherein
   each of R₁ and R₄ is independently selected from —H, halo, C₁₋₆ alkyl, and C₁₋₆ alkoxy, wherein the C₁₋₆ alkyl and C₁₋₆ alkoxy are optionally substituted with 1-3 of halo;
   R′₂ is —H and R₂ is selected from halo, hydroxy, optionally substituted C₁₋₆ alkyl, —OC(O)R^{A}, —O(SO₂)NH₂, —OCH(Rₘ)OC(O)Rₙ, —OCH(Rₘ)OP(O)(ORₙ)₂, —OP(O)(ORₙ)₂, and

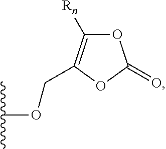

wherein R^{A} is selected from optionally substituted C₁₋₁₂ alkyl, optionally substituted C₆₋₁₂ aryl and optionally substituted C₅₋₁₂ heteroaryl, each Rₘ is independently optionally substituted C₁₋₁₂ alkyl, each Rₙ is independently selected from optionally substituted C₁₋₁₂ alkyl, optionally substituted C₃₋₈ cycloalkyl, and optionally substituted phenyl; or R₂ and R′₂ together form oxo;
R₃ is —H or optionally substituted C₁₋₃ alkyl; and
A is a phenyl, pyridin-2-yl, pyridin-3-yl, or pyridin-4-yl;
and wherein the MPC inhibitor and BCR-ABL kinase inhibitor are for administration together or separately.

The invention further provides the use of an MPC inhibitor in the manufacture of a medicament for:
(i) inhibiting expansion of the LSC population in CML; and/or
(ii) sensitising LSCs to treatment with a BCR-ABL kinase inhibitor in CML;
wherein the MPC inhibitor is a compound of Formula (I) or a pharmaceutically acceptable salt thereof:

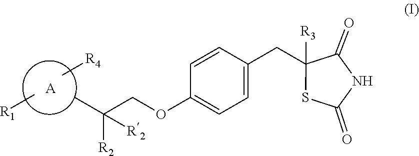

wherein
   each of R₁ and R₄ is independently selected from —H, halo, C₁₋₆ alkyl, and C₁₋₆ alkoxy, wherein the C₁₋₆ alkyl and C₁₋₆ alkoxy are optionally substituted with 1-3 of halo;
   R′₂ is —H and R₂ is selected from halo, hydroxy, optionally substituted C₁₋₆ alkyl, —OC(O)R^{A}, —O(SO₂)NH₂, —OCH(Rₘ)OC(O)Rₙ, —OCH(Rₘ)OP(O)(ORₙ)₂, —OP(O)(ORₙ)₂, and

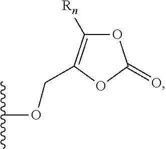

wherein R^{A} is selected from optionally substituted C₁₋₁₂ alkyl, optionally substituted C₆₋₁₂ aryl and optionally substituted C₅₋₁₂ heteroaryl, each Rₘ is independently optionally substituted C₁₋₁₂ alkyl, each Rₙ is independently selected from optionally substituted C₁₋₁₂ alkyl, optionally substituted C₃₋₈ cycloalkyl, and optionally substituted phenyl; or R₂ and R′₂ together form oxo;
R₃ is —H or optionally substituted C₁₋₃ alkyl; and
A is a phenyl, pyridin-2-yl, pyridin-3-yl, or pyridin-4-yl.

The medicament may be for use in conjunction with a BCR-ABL kinase inhibitor.

The invention further provides the use of an BCR-ABL kinase inhibitor in the manufacture of a medicament for use in the treatment of CML, wherein the BCR-ABL kinase inhibitor is for use in conjunction with an MPC inhibitor, and wherein the MPC inhibitor is a compound of Formula (I) or a pharmaceutically acceptable salt thereof:

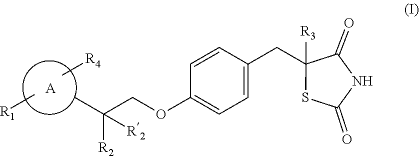

wherein
   each of R₁ and R₄ is independently selected from —H, halo, C₁₋₆ alkyl, and C₁₋₆ alkoxy, wherein the C₁₋₆ alkyl and C₁₋₆ alkoxy are optionally substituted with 1-3 of halo;
   R′₂ is —H and R₂ is selected from halo, hydroxy, optionally substituted C₁₋₆ alkyl, —OC(O)R^{A}, —O(SO₂)NH₂, —OCH(Rₘ)OC(O)Rₙ, —OCH(Rₘ)OP(O)(ORₙ)₂, —OP(O)(ORₙ)₂, and

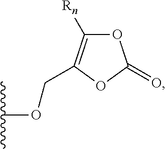

wherein R^{A} is selected from optionally substituted C₁₋₁₂ alkyl, optionally substituted C₆₋₁₂ aryl and optionally substituted C₅₋₁₂ heteroaryl, each Rₘ is independently optionally substituted C₁₋₁₂ alkyl, each Rₙ is independently selected from optionally substituted C₁₋₁₂ alkyl, optionally substituted C₃₋₈ cycloalkyl, and optionally substituted phenyl; or R₂ and R′₂ together form oxo;
R₃ is —H or optionally substituted C₁₋₃ alkyl; and
A is a phenyl, pyridin-2-yl, pyridin-3-yl, or pyridin-4-yl.

The BCR-ABL inhibitor may be an active site-binding inhibitor, i.e. an inhibitor which binds at or adjacent the active site of the kinase. It may inhibit binding of ATP to the ATP-binding site of the kinase, e.g. by competitive inhibition, typically by binding to the kinase at or near the ATP-binding site.

Examples of active site-binding inhibitors include imatinib (ST1571), nilotinib (AMN107), dasatinib (BMS-354825), bosutinib (SKI-606), ponatinib (AP24534) and bafetinib (NS-187).

Other BCR-ABL inhibitors have different mechanisms of action, such as the so-called STAMP (specifically targeting the ABL myristoyl pocket) inhibitors, which bind to the myristoyl binding pocket of ABL1.

Examples of STAMP inhibitors include asciminib (ABL001).

The MPC inhibitor is not an agonist of peroxisome proliferator-activated receptor gamma (PPAR-gamma), or has lower PPAR-gamma agonist activity than pioglitazone and/or rosiglitazone. For example, it may have at least a 10-fold reduced potency at PPAR-gamma as compared to pioglitazone and less than 50% of the full activation produced by rosiglitazone. Preferably the MPC inhibitor has no, or substantially no, PPAR-gamma agonist activity.

The MPC inhibitor is a compound of Formula (I).

In some embodiments of Formula (I):
R′₂ is —H and R₂ is selected from hydroxy and —OC(O)R^{A}; or
R₂ and R′₂ together form oxo.

It may be preferable that R₂ and R′₂ together form oxo.

In some embodiments:
R₁ is selected from C₁₋₆ alkyl optionally substituted with 1-3 of halo and C₁₋₆ alkoxy optionally substituted with 1-3 of halo; and
R₄ is —H.

In some embodiments, R₃ is —H.

The MPC inhibitor may be a compound of formula (IIa′) or (IIb′), or a pharmaceutically acceptable salt thereof:

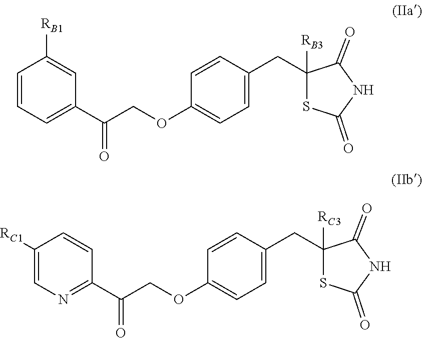

wherein
   R_{B1} and R_{C1} are independently selected from —H, halo, C₁₋₆ alkyl, and C₁₋₆ alkoxy, wherein the C₁₋₆ alkyl and C₁₋₆ alkoxy are optionally substituted with 1-3 of halo; and
   R_{B3} and R_{C3} are independently selected from —H and optionally substituted C₁₋₃ alkyl.

For example, the compound of Formula (I) may be selected from Compound A and Compound B:

| | |
|---|---|
| 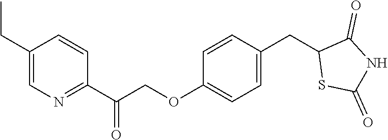 [SMILES: CCc1ccc(C(=O)COc2ccc(CC3SC(=O)NC3=O)cc2)nc1] | Compound A (MSDC-0160) |
| 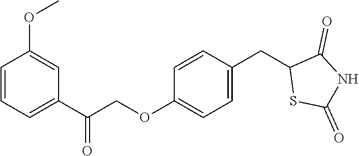 [SMILES: COc1cccc(C(=O)COc2ccc(CC3SC(=O)NC3=O)cc2)c1] | Compound B (MSDC-0602) |

The invention includes the combination of the aspects and preferred features described except where such a combination is clearly impermissible or expressly avoided.

### SUMMARY OF THE FIGURES

Embodiments and experiments illustrating the principles of the invention will now be discussed with reference to the accompanying figures.

FIG. 1. Human CML LSCs have increased glucose metabolism compared to HSCs

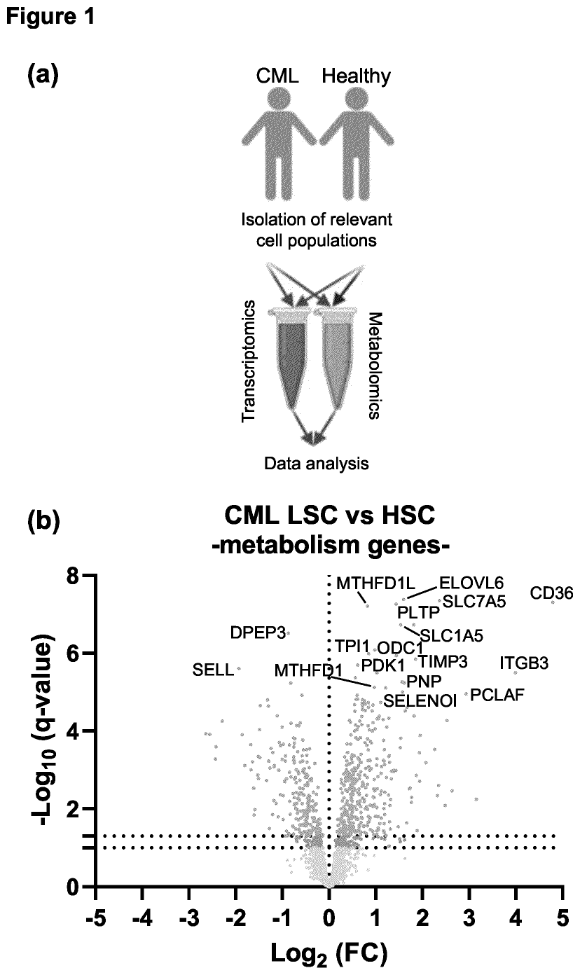

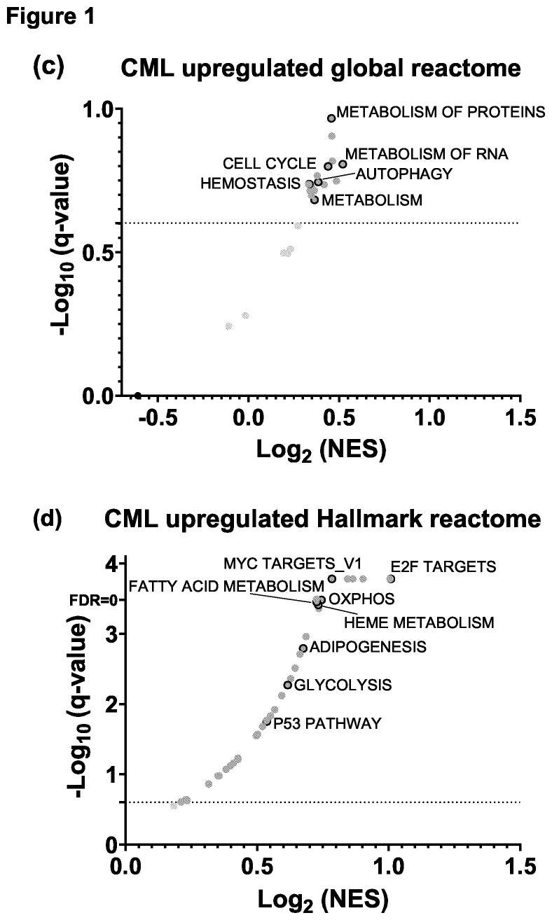

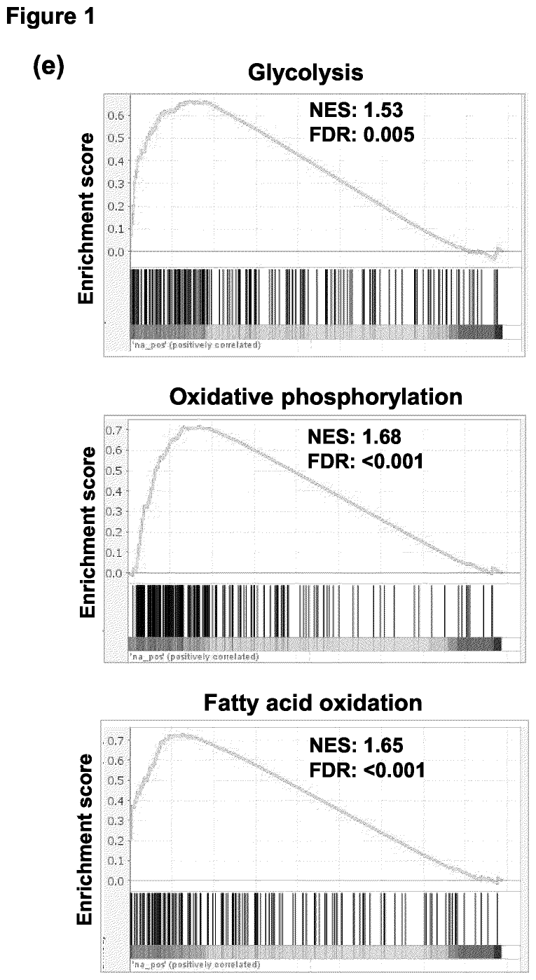

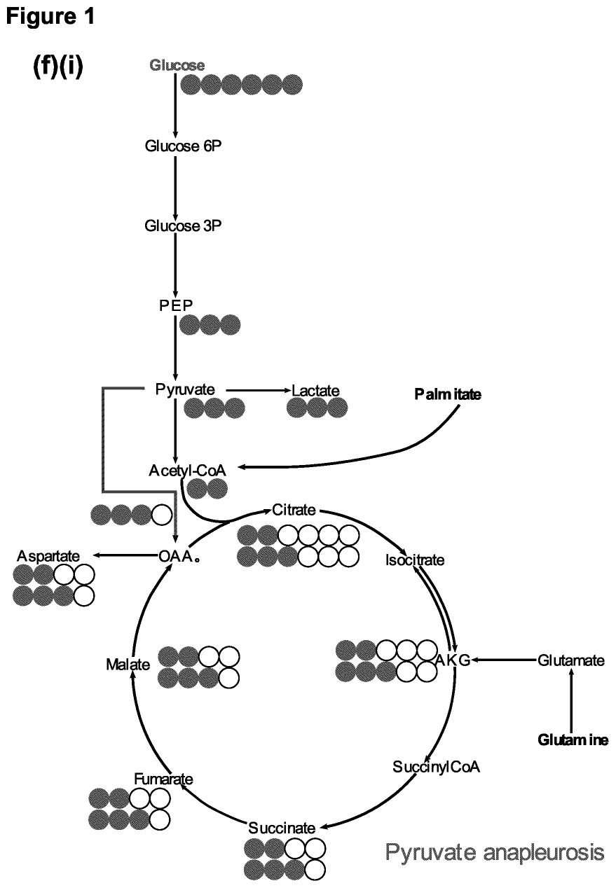

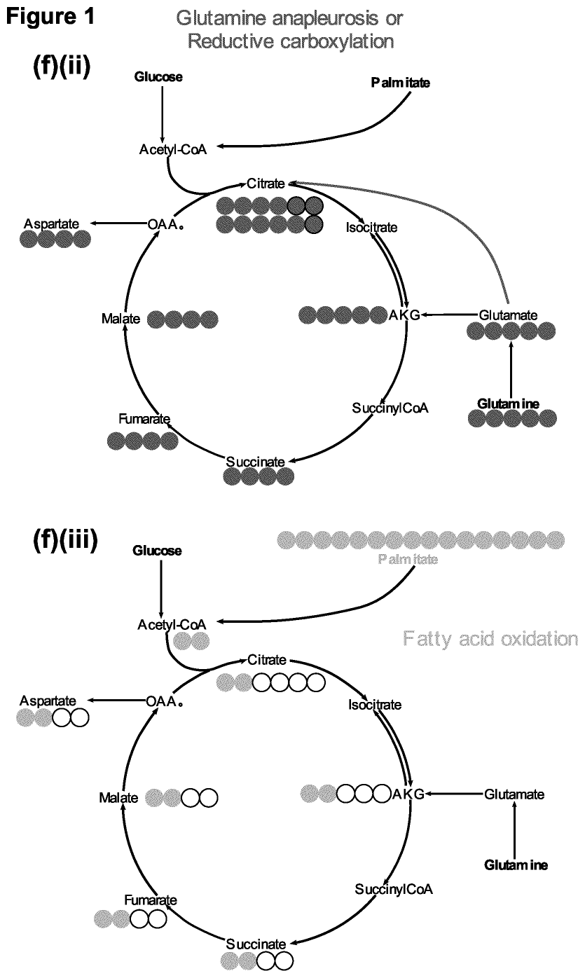

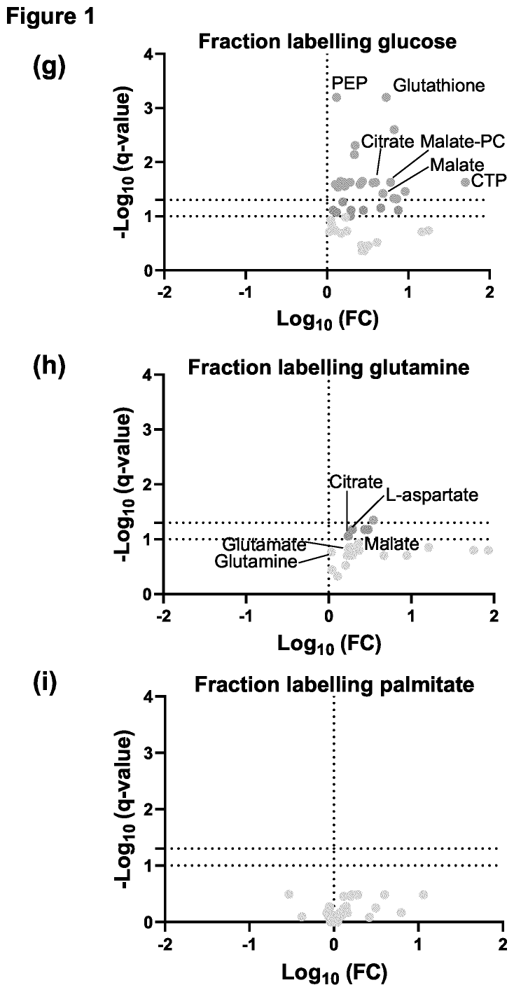

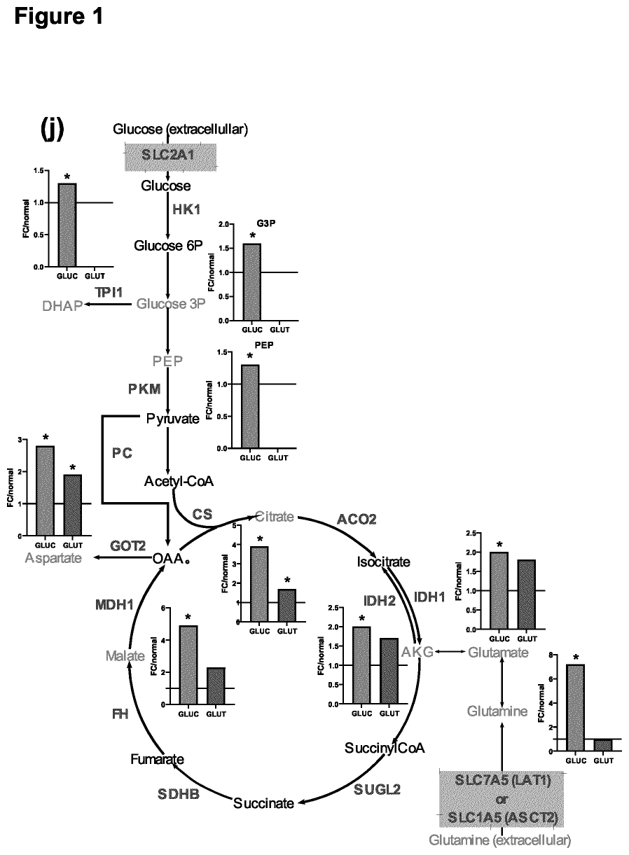

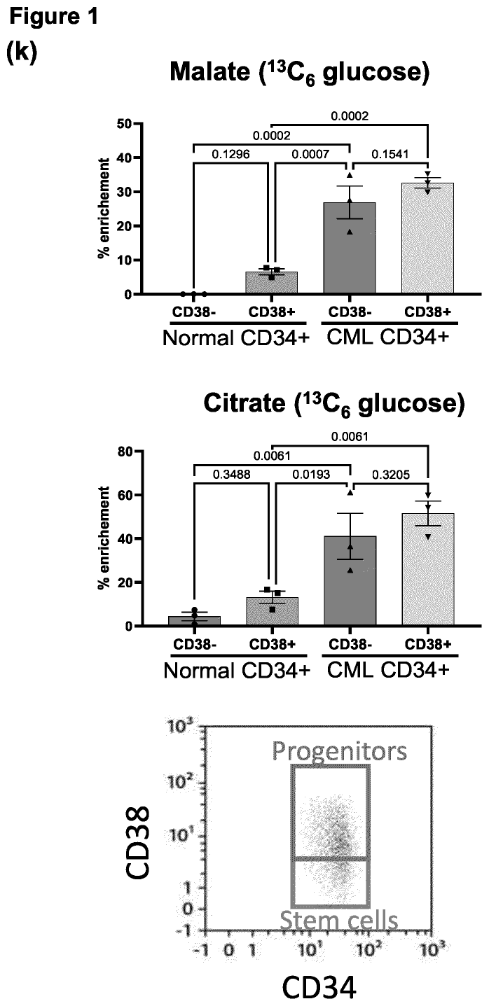

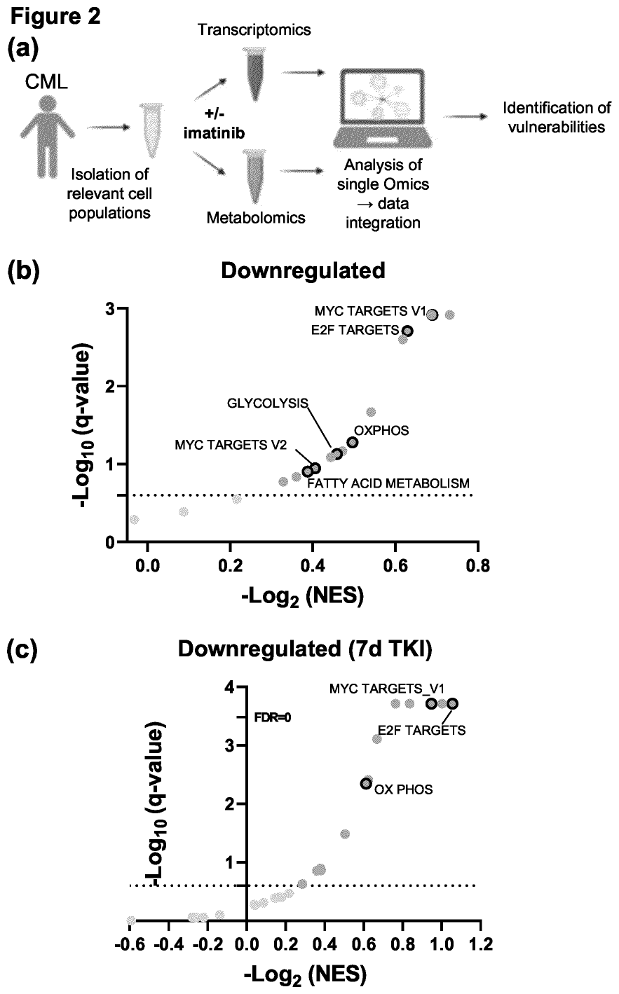

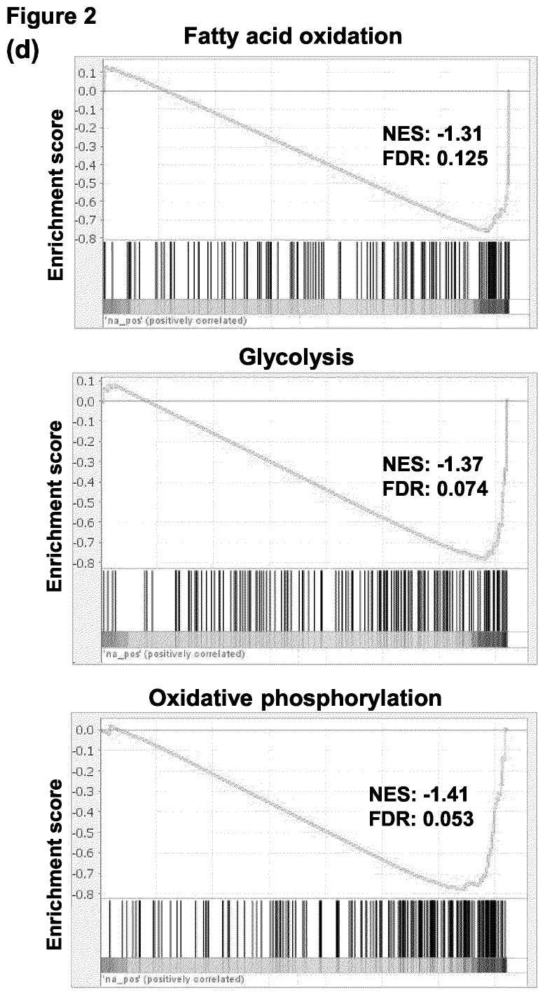

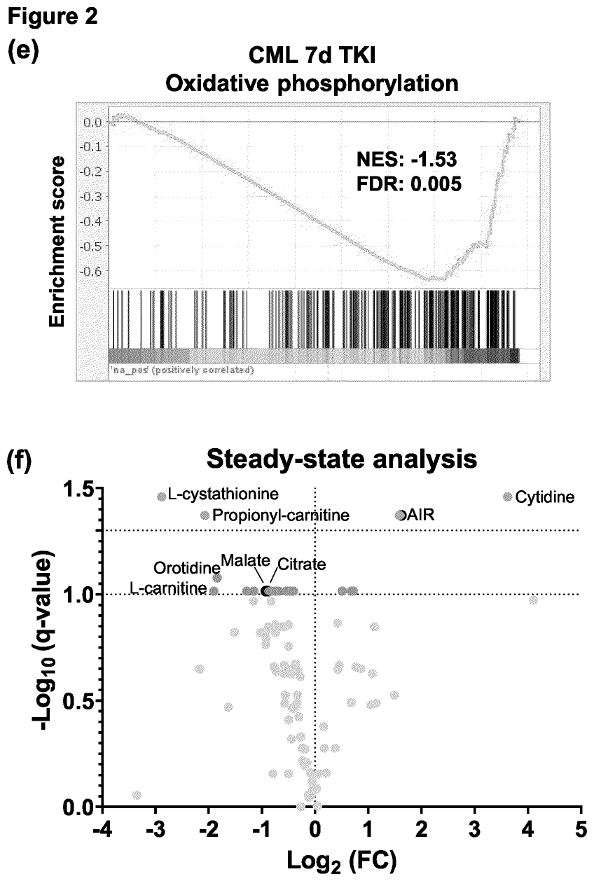

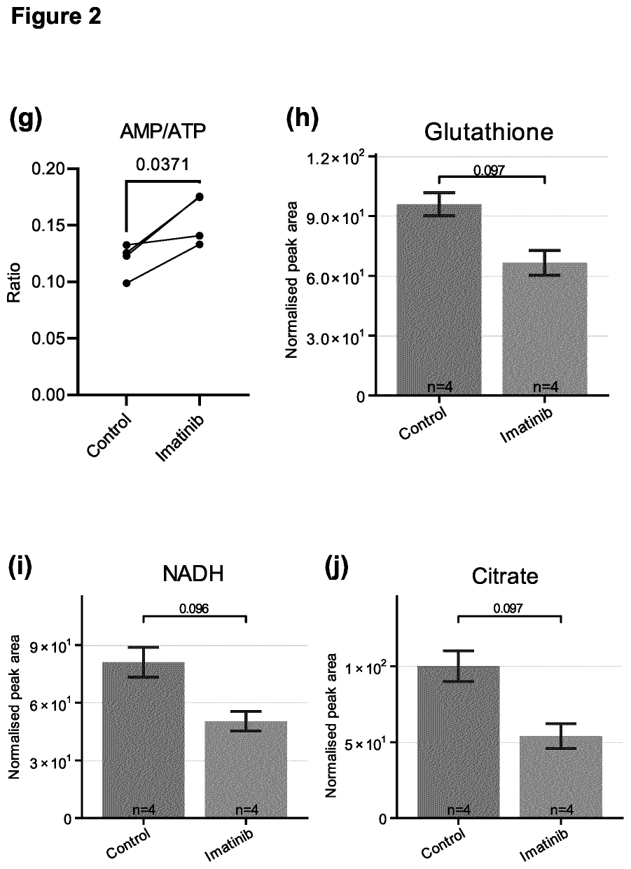

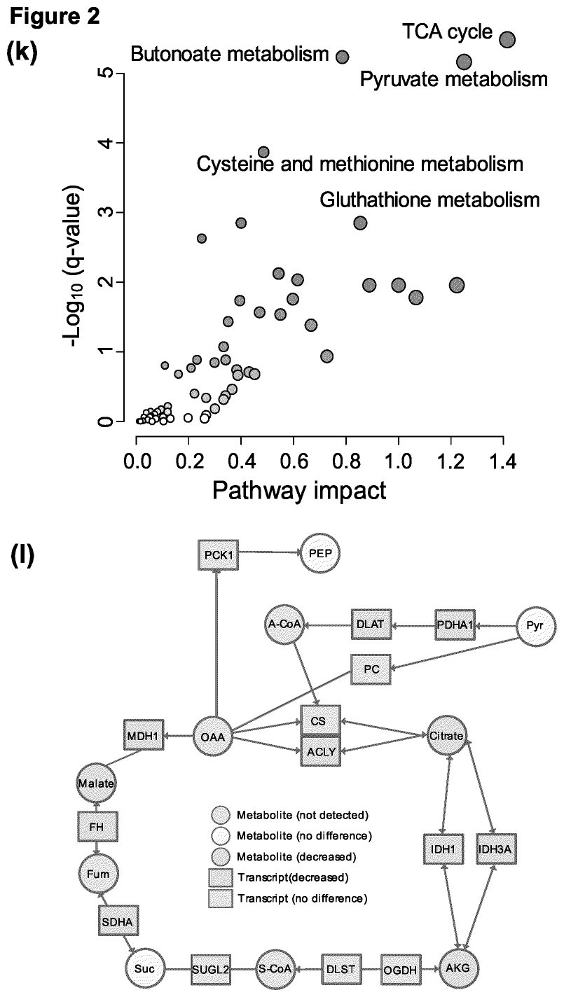

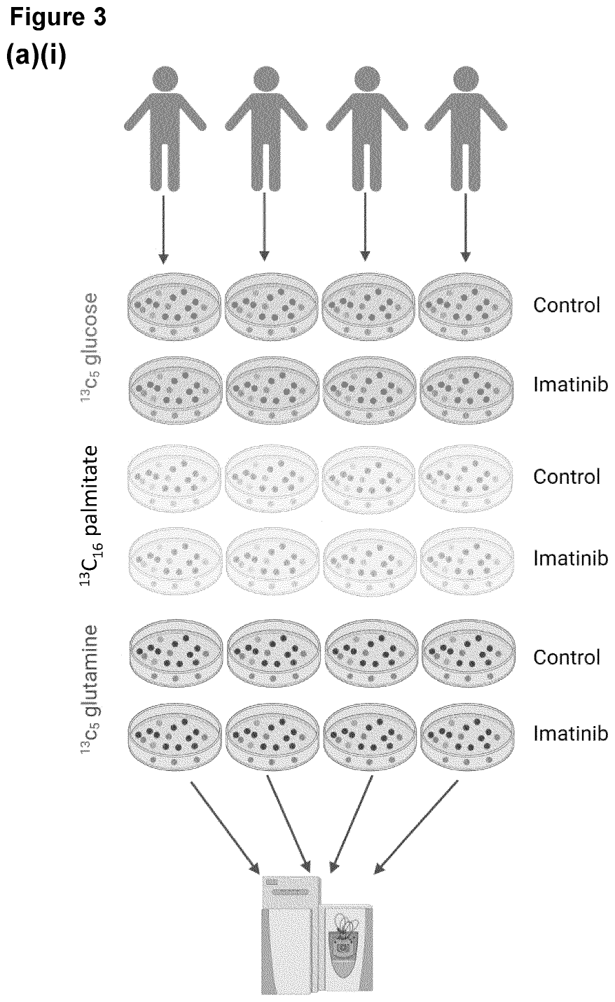

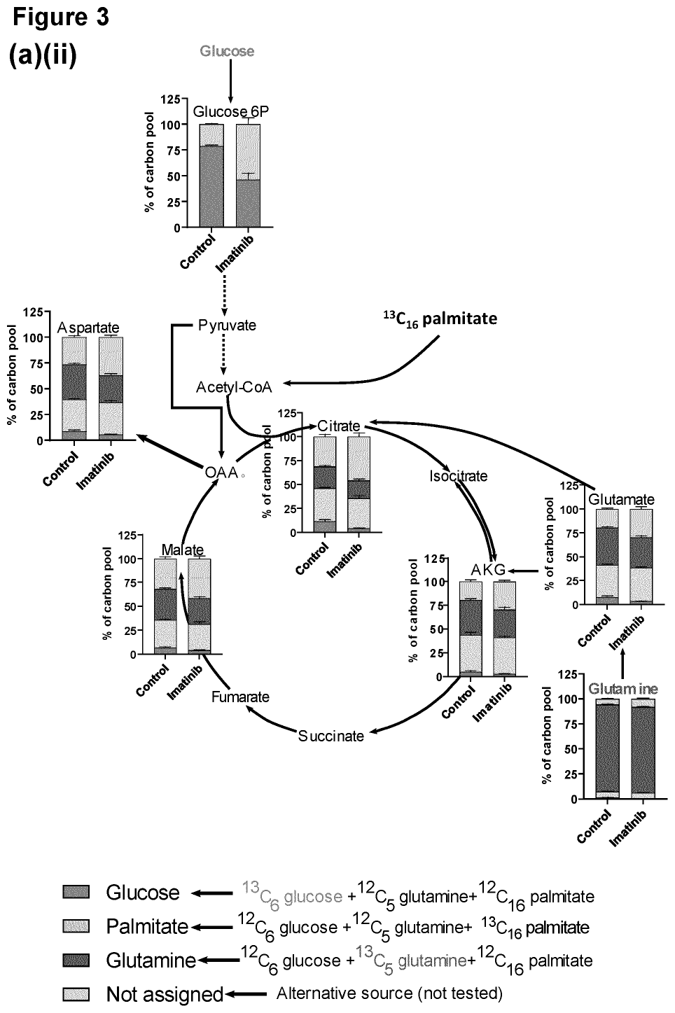

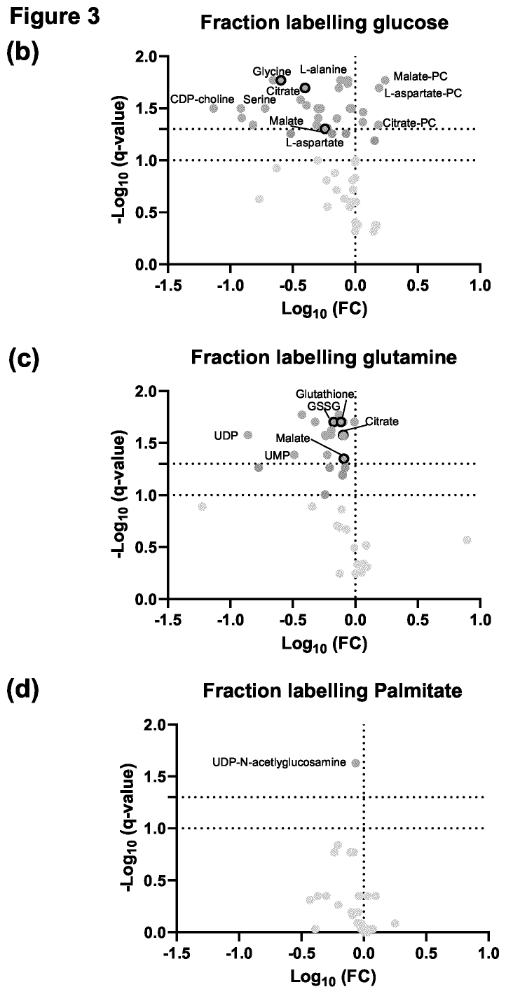

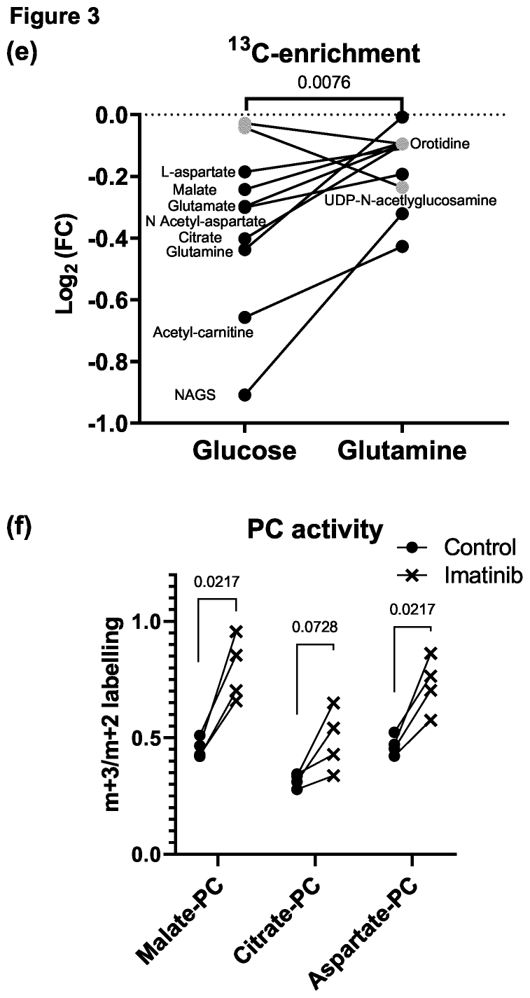

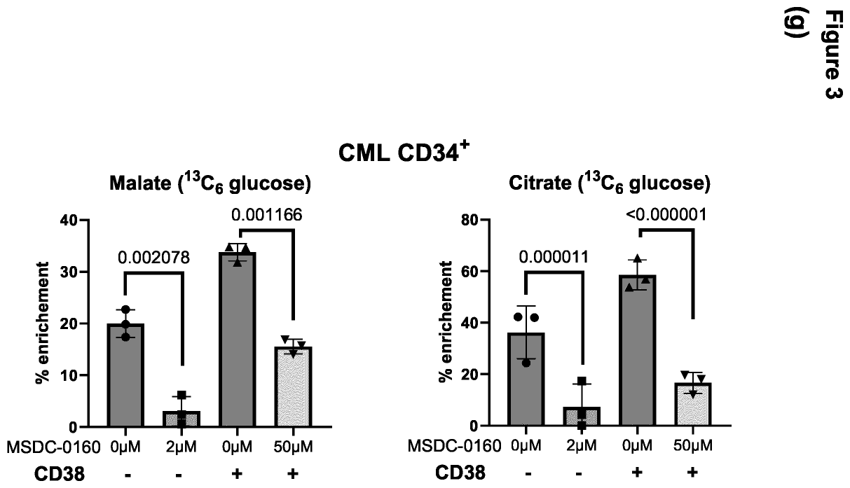

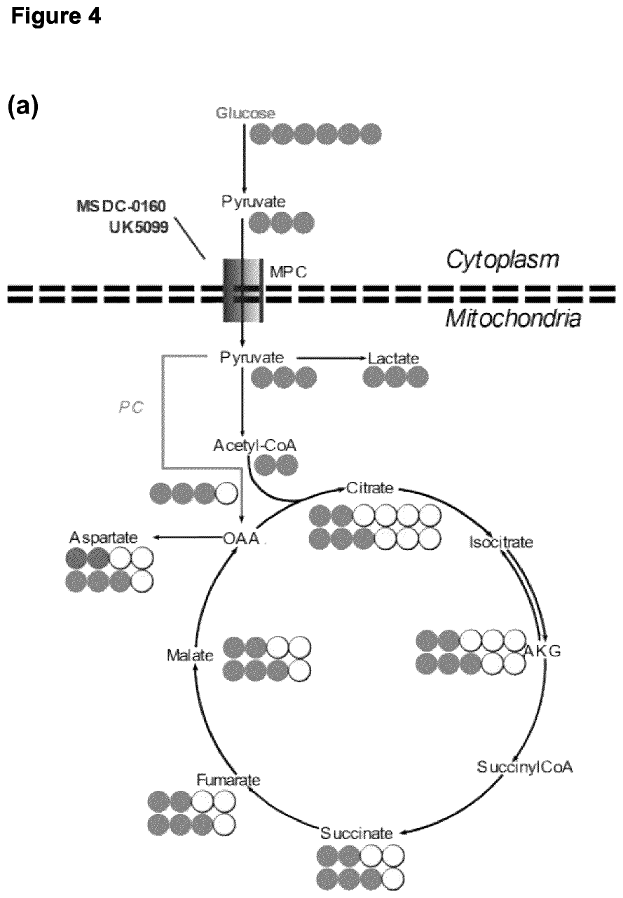

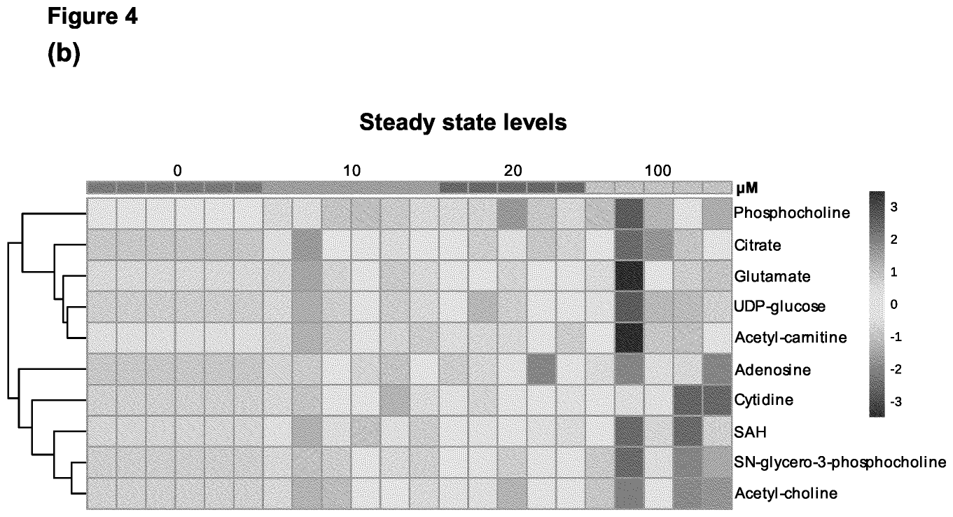

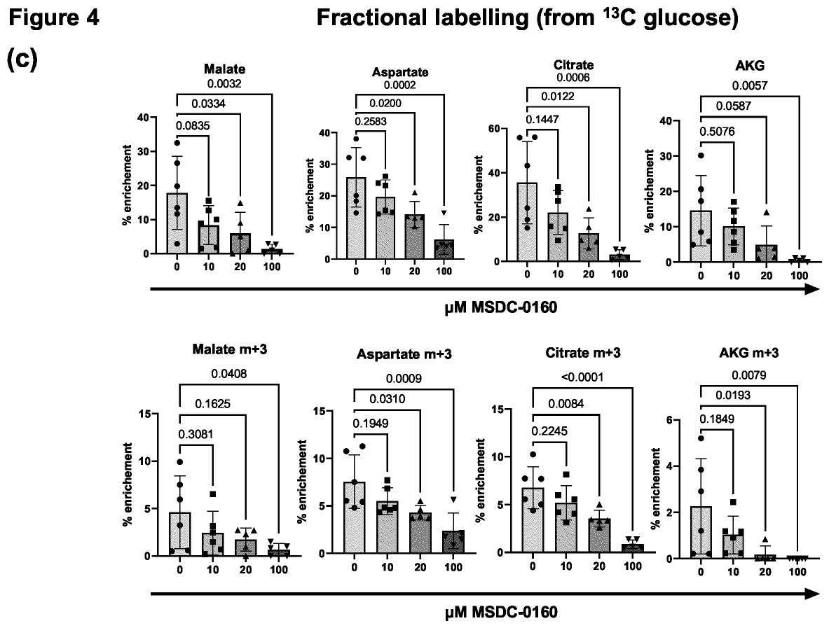

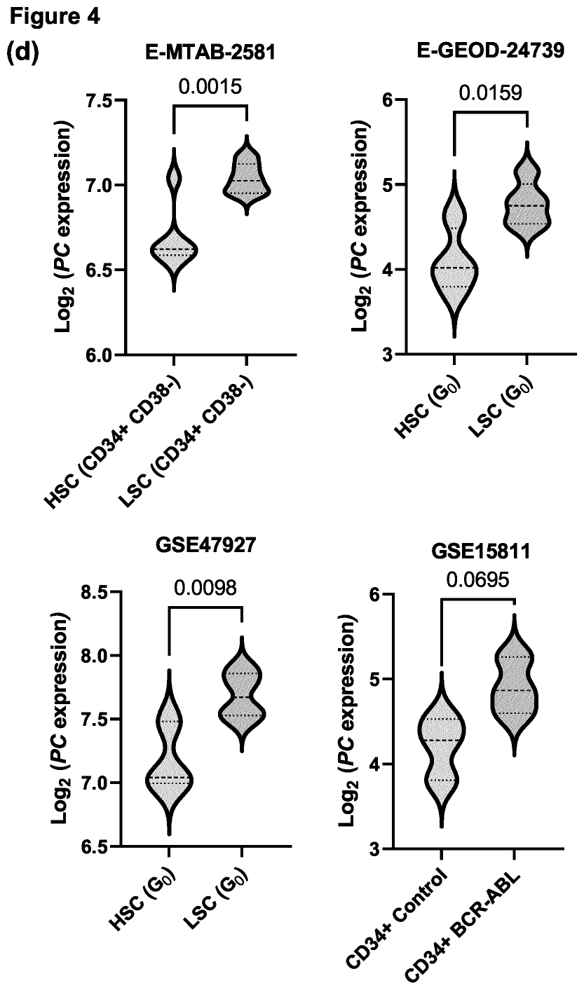

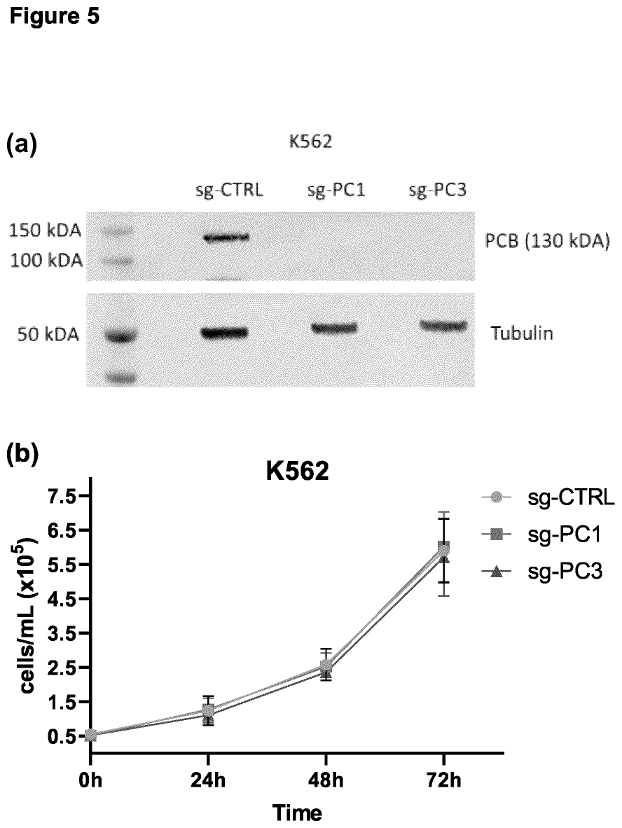

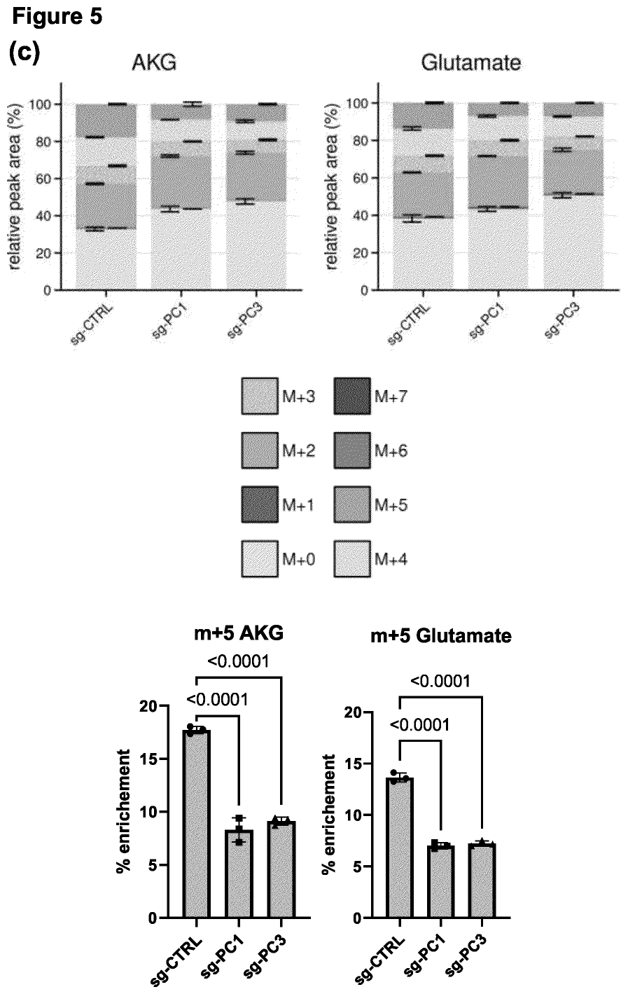

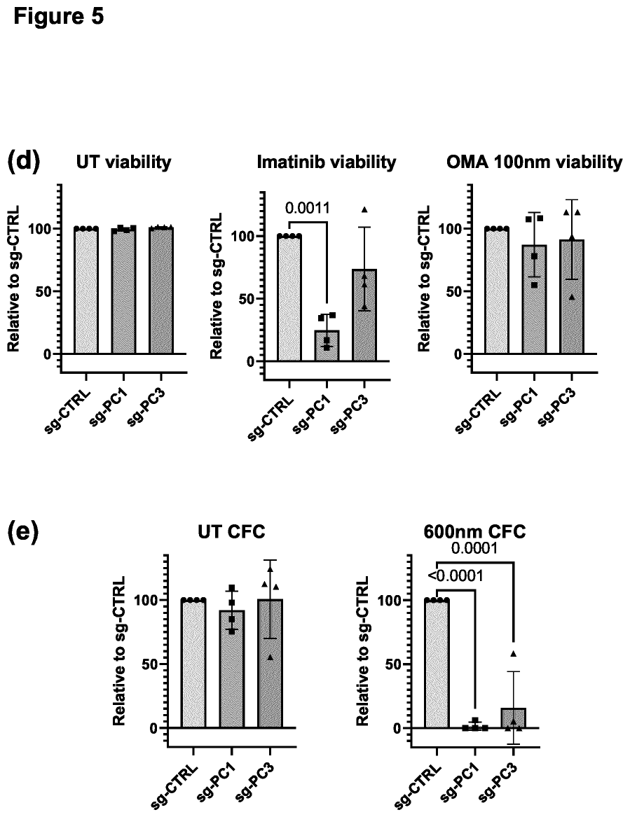

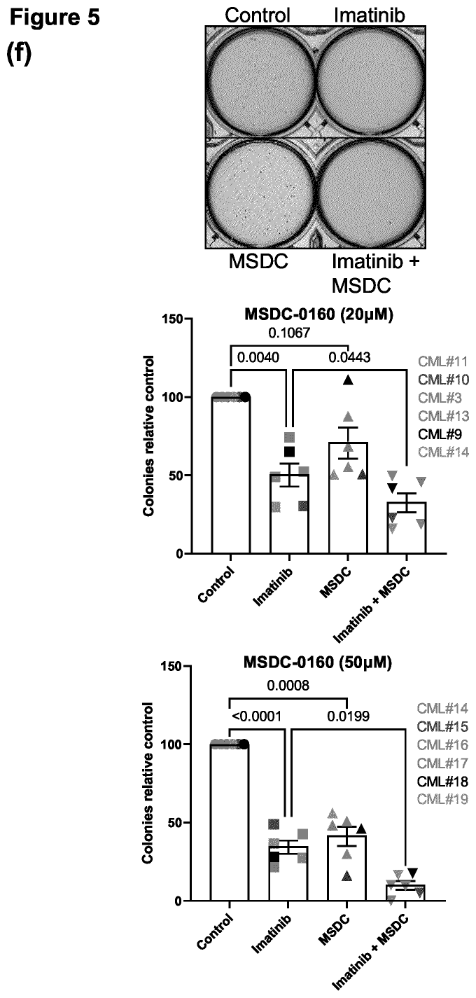

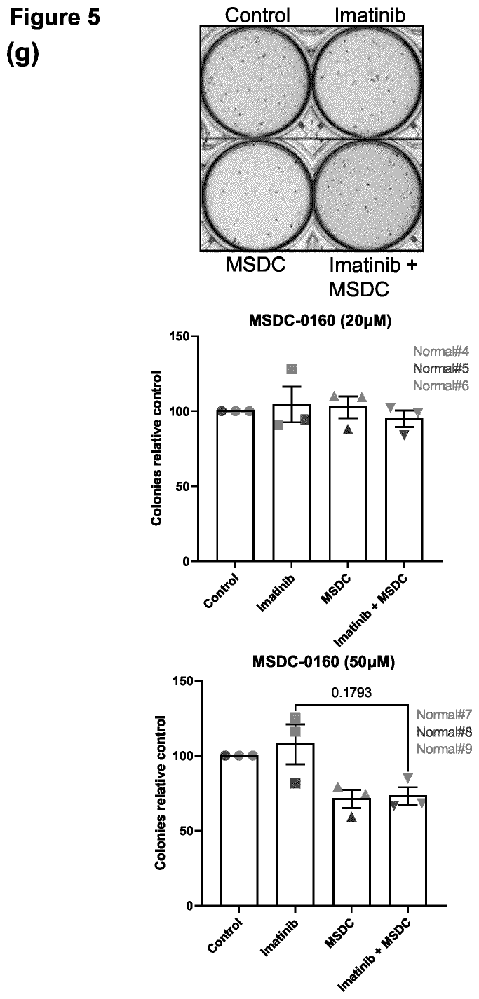

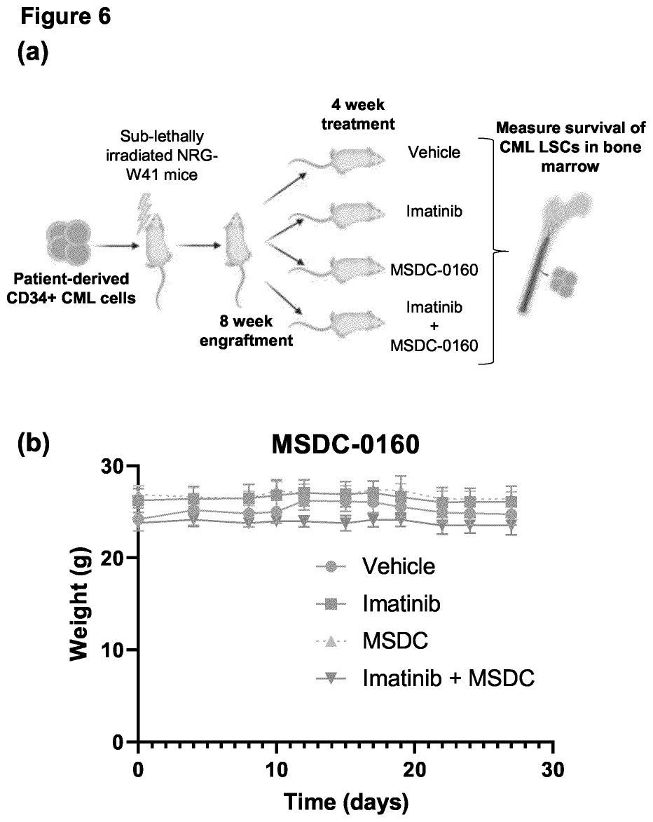

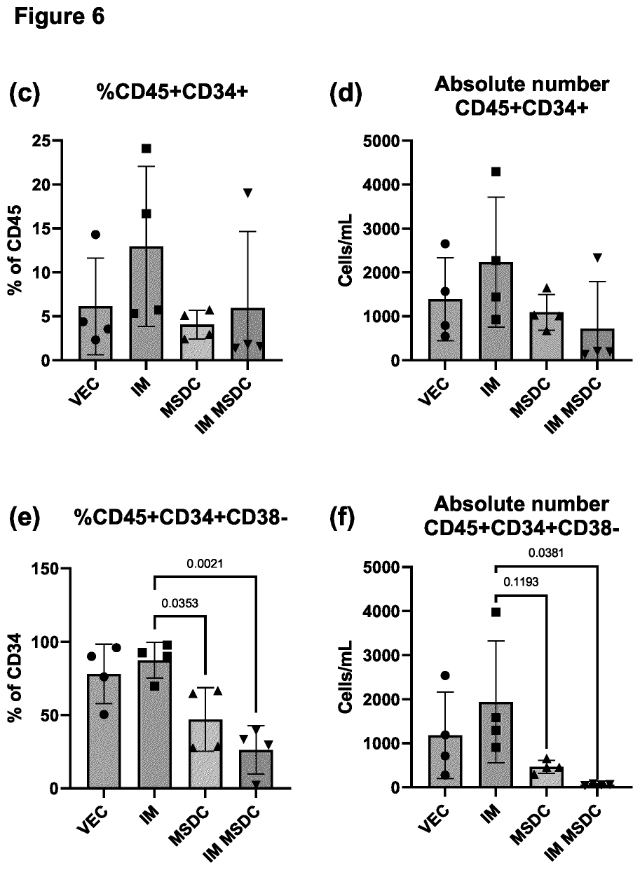

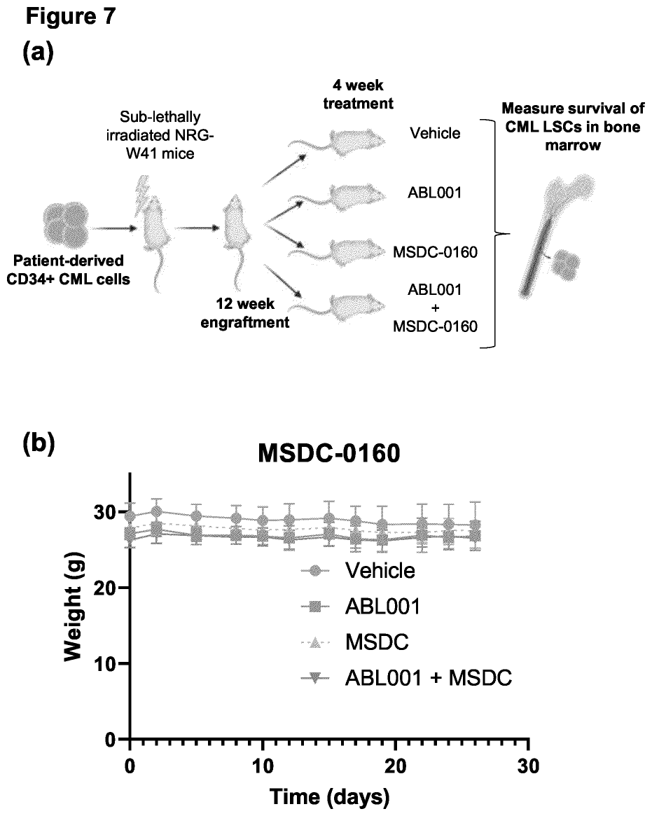

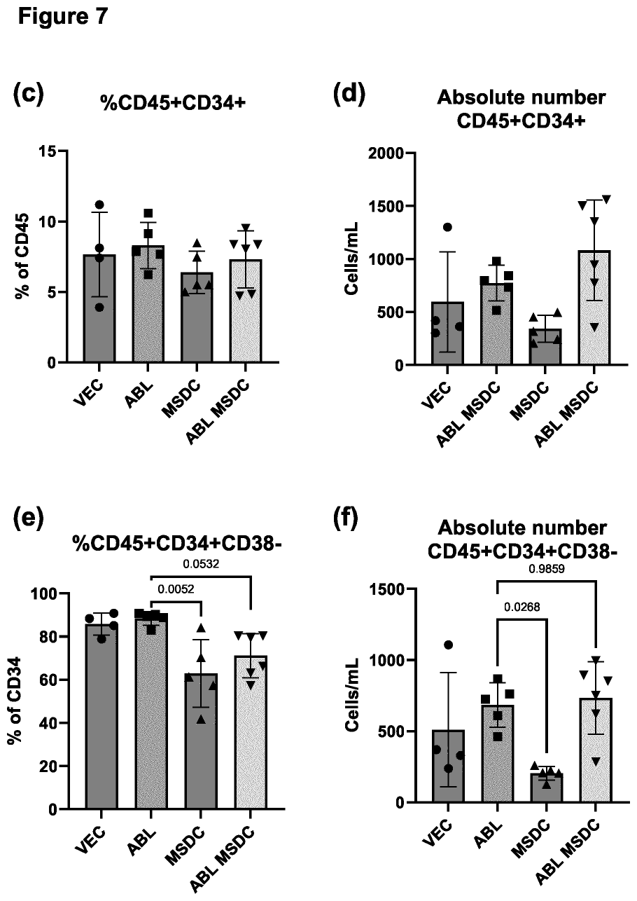

(a) Schematic overview of experimental design and omic dataset generation from human CML and non-leukaemic (healthy) samples. (b) Volcano plot of differentially expressed metabolism genes comparing CML LSCs and normal HSCs (CD34+CD38−). Red coloured genes (above upper horizontal dotted line) have a q value<0.05 and blue coloured genes (between horizontal dotted lines) have q value<0.1. The Benjamini-Hochberg adjustment was used to correct for multiple comparisons. (c) GSEA conducted using global Reactome pathway gene sets (see source data). (d) GSEA conducted using Hallmark gene sets. (e) CML-upregulated gene sets (from D) shown are glycolysis, oxidative phosphorylation and fatty acid oxidation. (f) Schematic overview of stable isotope labelling experiments. Tracers employed are ¹³C₆ glucose, ¹³C₅ glutamine and ¹³C₁₆ palmitate. (g)-(i) Volcano plots of metabolite fraction labelling following 24 hours culture in ¹³C₆ glucose, (n=4 each group; g), ¹³C₅ glutamine (n=6 CML, n=3 normal; h) and ¹³C₁₆ palmitate (n=3 for each group; i), comparing CML CD34+ and normal CD34+ cells. Red coloured metabolites (above upper horizontal dotted line) have q value<0.05 and blue coloured metabolites (between horizontal dotted lines) have a q value<0.1. Multiple unpaired t-tests were used and the two-stage step-up (Benjamini, Krieger, and Yekutieli) was used to correct for multiple comparisons. (j) Overlay of fold changes from G and H with transcriptomic data from FIG. 1B. Significant changes in gene expression are indicated by gene symbol in blue [SLC2A1, HK1, TPI1, PKM, PC, CS, ACO2, IDH1, IDH2, SUGL2, SDHB, FH, MDH1, GOT2, SLC7A5 (LAT1), SLC1A5 (ASCT2)]. Significant changes in fraction labelling are indicated by metabolite name in red [DHAP, Glucose 3P, PEP, Citrate, AKG, Malate, Aspartate, Glutamate, Glutamine, Glutamine (extracellular)]. GLUC (red bar) refers to fold change in glucose fraction labelling and GLUT (blue bar) refers to fold change in glutamine fraction labelling with average shown. * Refers to q value<0.1. (k) Fractional labelling (n=3 each normal or CML (and indicated subpopulations)) following 24 hours culture in ¹³C₆ glucose. Mean and SEM are plotted and an one-way ANOVA was used with multiple comparisons corrected with the Benjamini-Hochberg adjustment.

FIG. 2. Imatinib partially reverses BCR-ABL driven metabolic reprogramming of human CML LSCs

(a) Schematic overview of paired omic dataset generation from human CML cells (n=4) exposed to imatinib. CD34+ cells were treated in vitro with 2 μm imatinib for 48 hours. (b) GSEA conducted on data described in A using Hallmark gene sets. The down-regulated gene sets are shown. (c) GSEA conducted using Hallmark gene sets on CML CD34+ cells treated with imatinib for 7 days. The down-regulated gene sets are shown. (d) Imatinib-downregulated gene sets (from B), shown are fatty acid oxidation, glycolysis and oxidative phosphorylation. (e) Imatinib-downregulated gene sets (from C), shown is oxidative phosphorylation. (f) Volcano plot comparing metabolite steady state levels between control and imatinib treated CML CD34+ cells (n=4 patient samples, 2 independent experiments). Cells were treated with 2 μm imatinib for 48 hours. Red coloured metabolites (above upper horizontal dotted line) have q value<0.05 and blue coloured metabolites (between horizontal dotted lines) have a q value<0.1. Multivariate analysis was carried out using MetaboAnalyst 5.0. The Benjamini-Hochberg FDR method was used to correct for multiple comparisons. (g) AMP/ATP ratios are shown (n=4 patient samples). Statistical analysis performed using a paired t-test. (h)-(j) Selected metabolites from A with corresponding q values shown. Plots were generated using Autoplotter. Average and SEM are plotted. (k) Joint pathway analysis of transcriptomics and metabolomics data from control and imatinib treated CML CD34+ cells (n=4 patient samples, 2 μm imatinib for 48 hours). Analysis was carried out using MetaboAnalyst 5.0, and hypergeometric test, degree centrality and combined queries were selected. The Benjamini-Hochberg FDR method was used to correct for multiple comparisons. (I) The TCA cycle from F is shown and significant changes denoted in the key.

FIG. 3. Imatinib disrupts nutrient contribution to TCA cycle in CML CD34+ cells

(a) Left panel: Schematic overview of experimental design. Right panel: Fractional labelling (m+2 and above) to carbon pool from ¹³C₆ glucose, ¹³C₅ glutamine and ¹³C₁₆ palmitate in control and imatinib treated CML CD34+ cells (n=4 patients-derived cells per group). Average and SEM are plotted. Cells were treated with 2 μm imatinib for 48 hours. (b)-(d) Volcano plots of metabolite fraction labelling from ¹³C₆ glucose (b), ¹³C₅ glutamine (c) and ¹³C₁₆ palmitate (d) comparing control and imatinib treated CML CD34+ cells (n=4 patients-derived cells per group, 2 μm imatinib for 48 hours). Red coloured metabolites (above upper horizontal dotted line) have a q value<0.05 and blue coloured metabolites (between horizontal dotted lines) have a q value<0.1. Multiple paired t-tests were used and the two-stage step-up (Benjamini, Krieger, and Yekutieli) was used to correct for multiple comparisons. (e) Fold changes (imatinib/control) for fraction labelling from ¹³C₆ glucose and ¹³C₅ glutamine in CML CD34+ cells (n=4 patients-derived cells per group, 2 μm imatinib for 48 hours). In red are the two metabolites with larger decreases in ¹³C₅ glutamine contribution. Statistical analysis was performed using a paired t-test. (f) For relative PC/PDH activity CML LSCs (CD34+, n=4 patients-derived cells per group, 2 μm imatinib for 48 hours), the m+3/m+2 ratio is shown for the indicated metabolites. Multiple paired t-tests were used and the two-stage step-up (Benjamini, Krieger, and Yekutieli) was used to correct for multiple comparisons. (g) Fractional labelling from ¹³C₆ glucose for indicated cell fractions (n=3 each group, 2 μm imatinib for 24 hours). A two-way ANOVA was used for statistical analysis.

FIG. 4. CML CD34+ cells have increased PC activity that can be abrogated using MPC inhibitor

(a) Schematic of ¹³C₆ glucose conversion into ¹³C₃ pyruvate and its subsequent transport into mitochondria via MPC where it can be metabolised by pyruvate carboxylase or pyruvate dehydrogenase. Inhibition of MPC by UK-5099 or MSDC-0160 are indicated. (b) Steady state levels of significantly different metabolites (p<0.1) in CML CD34+ cells (n=5-6 patients-derived cell samples) exposed to indicated concentrations of MSDC-0160 for 24 hours. Analysis was carried out using MetaboAnalyst 5.0. A one-way ANOVA was performed, and the Benjamini-Hochberg FDR method was used to correct for multiple comparisons. (c) Individual patient samples from B are plotted for statistical analysis. Average and SEM are plotted. An ordinary one-way ANOVA was used with Dunnett's test used to correct for multiple comparisons. (d) Violin plots showing expression of pyruvate carboxylase (PC) in normal HSCs and CML LSCs in indicated datasets with stemness marker listed on X-axis. An unpaired t-test was used for statistical analysis.

FIG. 5. PC ablation or inhibition of mitochondrial pyruvate import sensitises CML to imatinib

(a) Western blot of PC and loading control (tubulin) from vector control or PC knockout K562 cells. (b) Proliferation of K562 cells from A. Shown is average and SEM from 3 independent experiments. (c) Fractional labelling from ¹³C₆ glucose into indicated metabolites of PC knockout cells are shown (n=3) after 24 hours. Plots were generated using Autoplotter, average and SD are plotted. Statistical analyses were conducted on the m+5 fraction. An ordinary one-way ANOVA was used with Dunnett's test used to correct for multiple comparisons. (d) Cells viability was measured using Annexin V and 7AAD. Cells were either left untreated (UT) or exposed for 72 hours to 600 nm imatinib or 100 nm omacetaxine (OMA). Shown is average and SEM from 4 independent experiments. An ordinary one-way ANOVA was used with Dunnett's test used to correct for multiple comparisons. (e) Colony Forming Cell (CFC) potential of control and PK knockout cells. Cells were either left untreated (UT) or exposed for 72 hours to 600 nm imatinib. Shown is average and SEM from 4 independent experiments. An ordinary one-way ANOVA was used with Dunnett's test used to correct for multiple comparisons. (f)-(g) Representative images of colonies and bar plots of colony numbers resulting from CML CD34+ cells (n=6 CML patients; f) or normal CD34+ cells (n=3 healthy donors; g) exposed to 2 μm imatinib, 20 μm or 50 μm MSSC-0160 or combination for 72 hours. Average and SEM are plotted. A repeated measure one-way ANOVA was used with multiple comparisons corrected for using Tukey's test.

FIG. 6. Combining inhibition of Mitochondrial pyruvate transport and BCR-ABL eradicates CML stem cells in vivo

(a) Schematic overview of experimental design for in vivo experiment. (b) Weights of mice taken over experiment. Average and SD are plotted. (c) and (e) The percentage of CML CD34+ cells (from CD45; c) or CML CD34+CD38−cells (from CD34+; e) is shown (n=4 mice per group). Average and SD are plotted. An ordinary one-way ANOVA was used with Dunnett's test used to correct for multiple comparisons. (d) and (f) The absolute number of CML CD34+ cells (d) or CML CD34+CD38−cells (f) is shown (n=4 mice per group). Average and SD are plotted. An ordinary one-way ANOVA was used with Dunnett's test used to correct for multiple comparisons.

FIG. 7. Combination of asciminib (ABL001) and MSDC-0160

(a) Schematic overview of experimental design for in vivo experiment. (b) Weights of mice taken over experiment. Average and SD are plotted. (c) and (e) The percentage of CML CD34+ cells (from CD45; c) or CML CD34+CD38−cells (from CD34+; e) is shown (n=4 mice per group). Average and SD are plotted. An ordinary one-way ANOVA was used with Dunnett's test used to correct for multiple comparisons. (d) and (f) The absolute number of CML CD34+ cells (d) or CML CD34+CD38−cells (f) is shown (n=4 mice per group). Average and SD are plotted. An ordinary one-way ANOVA was used with Dunnett's test used to correct for multiple comparisons.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

### CML

Chronic myeloid leukaemia (CML) is a myeloproliferative disorder characterised by unregulated proliferation of myeloid cells in the bone marrow and their accumulation in the blood. It is typically diagnosed in the chronic phase, which may be largely asymptomatic. The disease may progress from the chronic phase to the accelerated phase, and subsequently to the blast (or blast crisis) phase, which may be lethal.

CML is caused by a reciprocal translocation between chromosomes 9 and 22 t(9;22)(q34;q11) that leads to the formation of the so-called Philadelphia chromosome (22q-). The translocation generates a constitutively active gene fusion designated BCR-ABL, which places the ABL1 gene adjacent a portion of the BCR (breakpoint cluster region) gene. The size of the encoded fusion protein varies depending on the precise location of the recombination event, typically ranging from 185 kDa (p185) to 210 kDa (p210).

The BCR-ABL kinase (sometimes referred to as BCR-ABL1) contains domains from both ABL1 and BCR proteins. The wild type ABL1 protein contains a myristoylated cap region which interacts with a myristoyl binding site within ABL1 as an auto-regulatory measure. In the BCR-ABL fusion, this is replaced by a portion of the BCR protein, which renders the fusion protein constitutively active.

TKIs capable of targeting the BCR-ABL fusion (i.e. BCR-ABL inhibitors, discussed in more detail below) have become first-line therapy options for patients with CML. While a small fraction (˜10%) of patients respond well to TKI therapy and can be withdrawn from treatment following TKI monotherapy (i.e. they enter therapy-free remission), this is not possible for the large majority of patients.

This is believed to be due, at least in part, to the persistence of a population of primitive stem cells (leukemic stem cells: LSCs), which appear largely resistant to TKI therapy, and are able to re-establish the CML cell population in the blood after TKI treatment is withdrawn. The persistence of the LSC population also greatly increases the chance of developing resistance to the TKI therapy, e.g. by the acquisition of mutations in the BCR-ABL kinase which reduce or abrogate binding of the TKI. This has led to the development of next-generation TKIs specifically tailored to be effective against the more common BCR-ABL variants.

An LSC for the purposes of the invention can be considered to be a hematopoietic (CD34⁺CD38⁻) stem cell comprising a BCR-ABL fusion gene, typically as a result of a reciprocal translocation between chromosomes 9 and 22, and capable of expressing the BCR-ABL protein.

### BCR-ABL Inhibitors

A very considerable amount of work has been performed in the development of BCR-ABL inhibitors. A great many are known, and a number have achieved regulatory approval and are marketed for treatment of CML. Some of the best known are illustrated below but those in the field will be well aware of potential alternatives.

The majority of BCR-ABL inhibitors bind to the kinase at or near the active site, and may be designated “active site-binding inhibitors”. For example, they may bind at or near the ATP-binding site, and may inhibit binding of ATP to the kinase, e.g. competitively or semi-competitively. Certain examples of active site-binding inhibitors are shown below.

### Imatinib (ST1571):

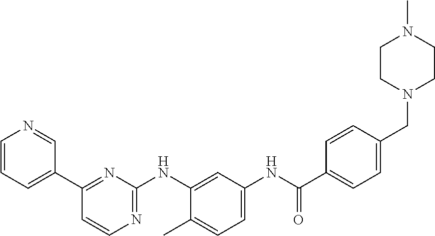 [SMILES: Cc1ccc(NC(=O)c2ccc(CN3CCN(C)CC3)cc2)cc1Nc1nccc(-c2cccnc2)n1]

Imatinib binds close to the ATP binding site, locking the kinase in a closed or self-inhibited conformation, and therefore inhibiting the enzyme activity of the protein semi-competitively. Many BCR-ABL mutations can cause resistance to imatinib by shifting its equilibrium toward the open or active conformation.

The mesylate salt may be particularly preferred.

### Nilotinib (AMN107):

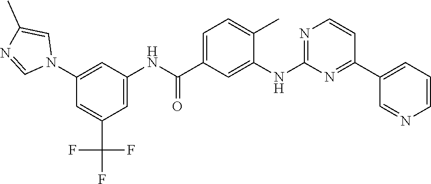 [SMILES: Cc1cn(-c2cc(NC(=O)c3ccc(C)c(Nc4nccc(-c5cccnc5)n4)c3)cc(C(F)(F)F)c2)cn1]

Nilotinib is structurally related to, but more potent than, imatinib.

The hydrochloride salt may be particularly preferred, especially in monohydrate form.

### Dasatinib (BMS-354825):

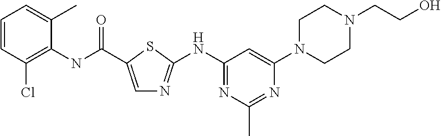

Dasatinib displays particularly strong binding to the BCR-ABL kinase compared to imatinib and nilotinib.

Consequently, while it has a relatively short plasma half-life, its duration of action is longer.

### Bosutinib (SKI-606):

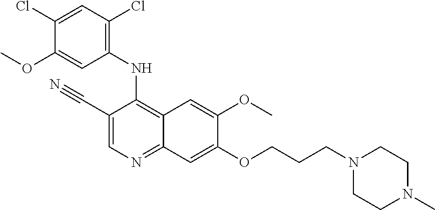

Bosutinib retains inhibitory activity against some, although not all, imatinib-resistant forms of BCR-ABL.

### Ponatinib (AP24534):

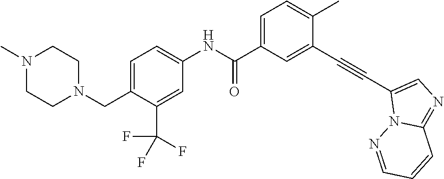 [SMILES: Cc1ccc(C(=O)Nc2ccc(CN3CCN(C)CC3)c(C(F)(F)F)c2)cc1C#Cc1cnc2cccnn12]

Ponatinib was designed using a computational and structure-based drug design platform to inhibit the enzymatic activity of BCR-ABL with high potency and broad specificity, and was intended to have activity against a variety of isoforms which are to treatment with other TKIs, including the T3151 mutation.

### Bafetinib (INNO-406):

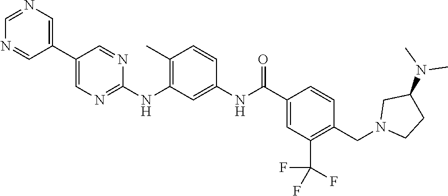 [SMILES: Cc1ccc(NC(=O)c2ccc(CN3CC[C@H](N(C)C)C3)c(C(F)(F)F)c2)cc1Nc1ncc(-c2cncnc2)cn1]

Bafetinib is effective against a number of imatinib-resistant isoforms of BCR-ABL (not including those having the T3151 mutation) and some dasatinib resistant mutations, and is also more specific for BCR-ABL than some other TKIs.

Other inhibitors have different mechanisms of action, such as binding to the ABL myristoyl pocket, so mimicking the auto-regulatory mechanism exerted by the myristoylated cap portion of the wild type ABL protein. Such inhibitors can be referred to as “STAMP” (specifically targeting the ABL myristoyl pocket) inhibitors. Examples include:

### Asciminib (ABL001):

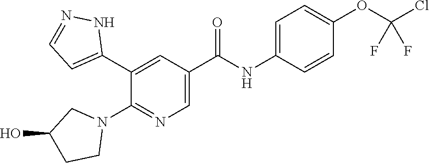 [SMILES: O=C(Nc1ccc(OC(F)(F)Cl)cc1)c1cnc(N2CC[C@@H](O)C2)c(-c2ccn[nH]2)c1]

The hydrochloride salt may be particularly preferred.

Since STAMP inhibitors have a different mechanism of action to the active site-binding inhibitors, they have different (typically non-overlapping) groups of resistance mutations.

It will be apparent that BCR-ABL inhibitors may be formulated and/or administered in the form of a pharmaceutically acceptable salt. Reference to any specific inhibitor should be taken to include pharmaceutically acceptable salt forms of that inhibitor unless the context demands otherwise.

### MPC Inhibitor

The inventors have found that pyruvate anaplerosis via pyruvate kinase (PC) and fatty acid oxidation in LSCs is unaffected by treatment with BCR-ABL inhibitors such as imatinib. They have further found that mitochondrial pyruvate carrier (MPC) inhibitors have a number of effects on the LSC population.

Firstly, they have a direct effect, inhibiting expansion of the LSC population. As noted above, the term “inhibiting expansion” in the context of the LSC population is used to encompass slowing of the rate of increase of the stem cell population as compared to the rate of increase in the absence of treatment, holding the population size steady, and also actively reducing the size of the stem cell population. Without wishing to be bound by theory, the MPC inhibitor may exert its effect on the LSC population by inhibiting LSC proliferation, inducing LSC differentiation (e.g. into a less primitive cell type, such as CML progenitor cells or fully differentiated myeloid CML cells) and/or inducing LSC death (e.g. by apoptosis).

Further, MPC inhibitors are capable of sensitising LSCs to BCR-ABL inhibitor treatment, thus expanding the clinical utility of TKIs in the majority of CML patients, and enabling the LSC population in CML to be targeted via a simple combination therapy.

Individually or together, these activities open up the prospect of completely eliminating the disease (i.e. providing therapy-free remission) from far more patients than is currently possible using TKI therapy alone.

The MPC inhibitor may be a thiazolidinedione (“glitazone”) compound. Pioglitazone has previously been proposed for use in treatment of CML. See, for example, Rousselot et al., Cancer 2017 May 15; 123(10):1791-1799 (doi: 10.1002/cncr.30490); Prost et al., Nature 2015 Sep. 17;525(7569):380-3. (doi: 10.1038/nature15248); Pagnano et al., Am. J. Hematol. 2020 December; 95(12):E321-E323 (doi: 10.1002/ajh.25986). However, the effects observed for pioglitazone were attributed to its principal activity as a peroxisome proliferator-activated receptor gamma (PPAR-gamma) agonist, with no suggestion that an effect on pyruvate metabolism might be involved.

So-called “PPAR-sparing” thiazolidinedione compounds, having MPC inhibitory activity but reduced or no activity on PPAR-gamma, are described extensively in (for example) WO 2007/109024, WO 2007/109037, WO 2011/017244, WO 2010/105048, WO 2012/178142, WO 2014/093114, WO 2015/013187, U.S. Pat. Nos. 8,629,159, 9,155,729, 8,912,335, 9,126,959, 8,067,450 and 8,304,441.

Thus the MPC inhibitor is a compound of Formula (I) or a pharmaceutically acceptable salt thereof:

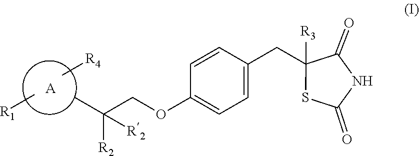

wherein
   each of R₁ and R₄ is independently selected from —H, halo, C₁₋₆ alkyl, and C₁₋₆ alkoxy, wherein the C₁₋₆ alkyl and C₁₋₆ alkoxy are optionally substituted with 1-3 of halo;
   R′₂ is —H and R₂ is selected from halo, hydroxy, optionally substituted C₁₋₆ alkyl, —OC(O)R^{A}, —O(SO₂)NH₂, —OCH(Rₘ)OC(O)Rₙ, —OCH(Rₘ)OP(O)(ORₙ)₂, —OP(O)(ORₙ)₂, and

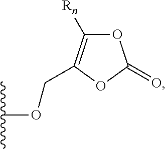

wherein R^{A} is selected from optionally substituted C₁₋₁₂ alkyl, optionally substituted C₆₋₁₂ aryl and optionally substituted C₅₋₁₂ heteroaryl, each Rₘ is independently optionally substituted C₁₋₁₂ alkyl, each Rₙ is independently selected from optionally substituted C₁₋₁₂ alkyl, optionally substituted C₃₋₈ cycloalkyl, and optionally substituted phenyl; or R₂ and R′₂ together form oxo;
R₃ is —H or optionally substituted C₁₋₃ alkyl; and
A is a phenyl, pyridin-2-yl, pyridin-3-yl, or pyridin-4-yl.

R₁ and R₄ are preferably independently selected from —H, fluoro, chloro, C₁₋₆ alkyl optionally substituted with 1-3 of halo, and C₁₋₆ alkoxy optionally substituted with 1-3 of halo.

In some embodiments R₁ and R₄ are independently selected from C₁₋₆ alkyl and C₁₋₆ alkoxy, either of which may be substituted with 1-3 halo groups. A halo substituent, where present, may be bonded to any carbon atom in the C₁₋₆ alkyl or C₁₋₆ alkoxy group. Where two or more halo substituents are present, they may be bonded to the same carbon atom or they may be bonded to different carbon atoms such as in a C₂₋₆ alkyl or C₂₋₆ alkoxy group.

In some embodiments R₁ and R₄ are independently C₁₋₆ alkyl, such as C₁₋₄ alkyl, that is optionally substituted with 1, 2 or 3 halo groups. Examples include methyl, ethyl, —CF₃, —CHF₂, and —CH₂F. A C₁₋₆ alkyl group may be straight or branched. Preferably, the C₁₋₆ alkyl group is unsubstituted.

In some embodiments R₁ and R₄ are independently C₁₋₆ alkoxy, such as C₁₋₄ alkoxy. Examples include methoxy, ethoxy, propoxy (—O-n-propyl), isopropoxy (—O-isopropyl), butoxy (—O-n-butyl), or tert-butoxy (—O-tert-butyl). These alkoxy groups may be substituted with 1-3 halo groups, such as where R₁ and R₄ are independently —OCHF₂ or OCF₃. A C₁₋₆ alkylene moiety in a C₁₋₆ alkoxy group may be straight or branched. Preferably, the C₁₋₆ alkoxy group is unsubstituted.

In preferred embodiments R₁ and R₄ are independently selected from —H, methyl, methoxy, ethyl, ethoxy, —O-isopropyl, —CF₃, —OCHF₂ and —OCF₃. Preferably, R₁ and R₄ are independently selected from methyl, ethyl, methoxy and ethoxy, and of these, ethyl and methoxy may be preferred.

R₁ and R₄ may be the same, or R₁ and R₄ may be different. For example, R₁ and R₄ may be the same and may both be —H, methyl, ethyl, methoxy or ethoxy. Preferably, one of R₁ and R₄ is —H, and the other of R₁ and R₄ is selected from halo, C₁₋₆ alkyl, and C₁₋₆ alkoxy, wherein the C₁₋₆ alkyl or C₁₋₆ alkoxy group is optionally substituted with 1-3 of halo.

In some embodiments R₁ is selected from C₁₋₆ alkyl optionally substituted with 1-3 of halo and C₁₋₆ alkoxy optionally substituted with 1-3 of halo; and R₄ is —H.

In some embodiments R₁ is selected from —H, methyl, methoxy, ethyl, ethoxy, —O-isopropyl, —CF₃, —OCHF₂, —OCF₃, and halo such as fluoro or chloro. R₁ may be selected from C₁₋₆ alkyl and C₁₋₆ alkoxy, which are optionally substituted with 1-3 halo groups. In preferred embodiments, R₁ is selected from methyl, ethyl, methoxy and ethoxy. More preferably, R₁ is selected from ethyl and methoxy.

In some embodiments R₄ is selected from —H, methyl, methoxy, ethyl, ethoxy, —O-isopropyl, —CF₃, —OCHF₂, —OCF₃ and halo such as fluoro or chloro. R₄ may be selected from C₁₋₆ alkyl and C₁₋₆ alkoxy, which are optionally substituted with 1-3 halo groups. In preferred embodiments, R₄ is —H.

In some embodiments, R₁ and R₄ may independently be C₁₋₆ alkoxy, such as where R₁ and R₄ are both methoxy or where R₁ and R₄ are both ethoxy. In other embodiments R₁ and R₄ may both be —H.

In some embodiments, R′₂ is H and R₂ is selected from
halo, such as fluoro or chloro;
hydroxy;
optionally substituted C₁₋₆ alkyl, such as methyl, CF₃, ethyl, iso-propyl, or tert-butyl;
—OC(O)R^{A}, such as —O-acetyl, —O-propanoyl, —O-butanoyl, —O-iso-butyryl, —O-n-pentanoyl, —O-pivaloyl, —O-hexanoyl, —O-succinoyl, —O-benzoyl, —O-naphthoyl, —O-imidazolyl, —O-thiazoloyl, or —O-pyridinoyl;
—O(SO₂)NH₂;
—OCH(Rₘ)OC(O)Rₙ;
—OCH(Rₘ)OP(O)(ORₙ)₂;
—OP(O)(ORₙ)₂, such as —OP(O)(OMe)₂, —OP(O)(OEt)₂, —OP(O)(O-ⁱPr)₂; and

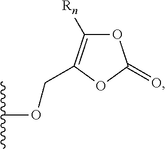

where R^{A} is selected from optionally substituted C₁₋₁₂ alkyl, optionally substituted C₆₋₁₂ aryl and optionally substituted C₅₋₁₂ heteroaryl, each Rₘ is independently an optionally substituted C₁₋₆ alkyl, each Rₙ is independently selected from optionally substituted C₁₋₁₂ alkyl, optionally substituted C₃₋₈ cycloalkyl, and optionally substituted phenyl.

When R′₂ is H and R₂ is as defined herein, the compound of Formula (I) may be in an R configuration or the S configuration. The compound of Formula (I) may be a single enantiomer, such as a compound of Formula (I′) or (I″). In some embodiments the compound of Formula (I) is a racemic mixture.

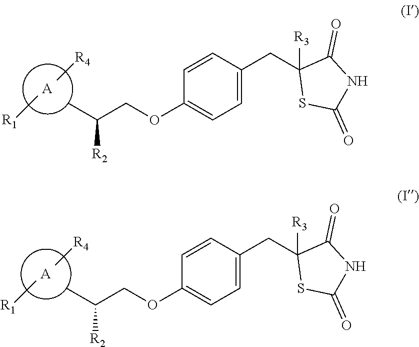

R₂ may be an optionally substituted C₁₋₆ alkyl group, which may be straight or may be branched. Examples of an optionally substituted C₁₋₆ alkyl include an optionally substituted C₁₋₄ alkyl group, such as an optionally substituted C₁₋₃ alkyl group. Examples of an optionally substituted C₁₋₆ alkyl group include methyl, CF₃, ethyl, iso-propyl, n-butyl, tert-butyl, n-pentyl and n-butyl;

An optionally substituted group as described herein may be substituted with one or more substituents selected from:
hydroxy;
halo, such as fluoro, chloro or bromo;
carboxy (COOH);
—OC(O)R^{A}, where R^{A} is selected from optionally substituted C₁₋₁₂ alkyl, optionally substituted C₆₋₁₂ aryl and optionally substituted C₅₋₁₂ heteroaryl, such as —O-acetyl, —O-propanoyl, —O-butanoyl, —O-iso-butyryl, —O-n-pentanoyl, —O-pivaloyl, —O-hexanoyl, —O-succinoyl, —O-benzoyl, —O-naphthoyl, —O-imidazolyl, —O-thiazoloyl, or —O-pyridinoyl.

Preferably, when a group is substituted the substituent is one or more of hydroxy, halo and carboxy.

In some embodiments R₂ is a substituted C₁₋₆ alkyl group, such as a substituted methyl group, such as CF₃. In other embodiments R₂ is unsubstituted C₁₋₆ alkyl.

R₂ may be selected from methyl, ethyl, propyl, iso-propyl, butyl, tert-butyl, pentyl, and hexyl, each of which is optionally substituted with hydroxy. Preferably, R₂ is methyl or ethyl, each of which is optionally substituted with hydroxy.

R₂ may be —OC(O)R^{A}, where R^{A} is selected from optionally substituted C₁₋₁₂ alkyl, optionally substituted C₆₋₁₂ aryl and optionally substituted C₅₋₁₂ heteroaryl. Preferably, R^{A} is selected from the following, which may each be optionally substituted:
C₁₋₆ alkyl, such as C₁₋₄ alkyl, such as C₁₋₃ alkyl;
phenyl or naphthyl, such as phenyl;
C₅₋₉ heteroaryl; such as furyl, pyrrolyl, thiazolyl, tetrazolyl, pyridyl, indolyl and indazolyl.

In some embodiments R₂ is —OCH(Rₘ)OC(O)Rₙ, —OCH(Rₘ)OP(O)(ORₙ)₂, —OP(O)(ORₙ)₂ or

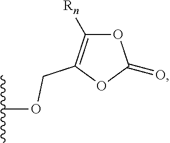

Where each Rₘ is independently an optionally substituted C₁₋₁₂ alkyl, and each Rₙ is independently selected from optionally substituted C₁₋₁₂ alkyl, optionally substituted C₃₋₈ cycloalkyl, and optionally substituted phenyl.

Rₘ is preferably an optionally substituted C₁₋₆ alkyl group, such as methyl, ethyl, —CF₃ or —CHF₂.

Rₙ is preferably selected from methyl, ethyl, iso-propyl, n-propyl, n-butyl, tert-butyl, cyclopropyl, cyclopentyl, cyclohexyl, and phenyl, each of which may be substituted such as with a substituent as described herein.

When R₂ is —OCH(Rₘ)OC(O)Rₙ, it may be selected from:

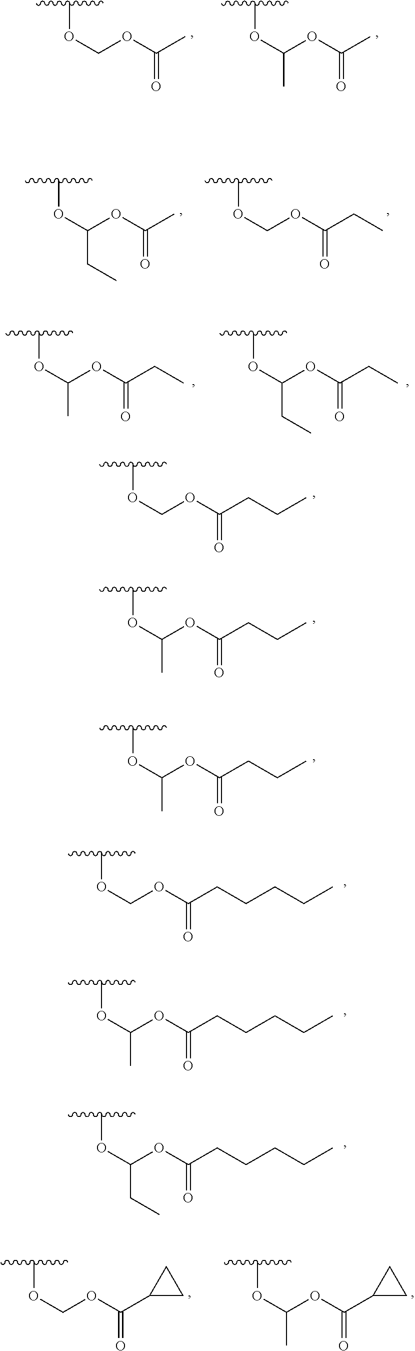

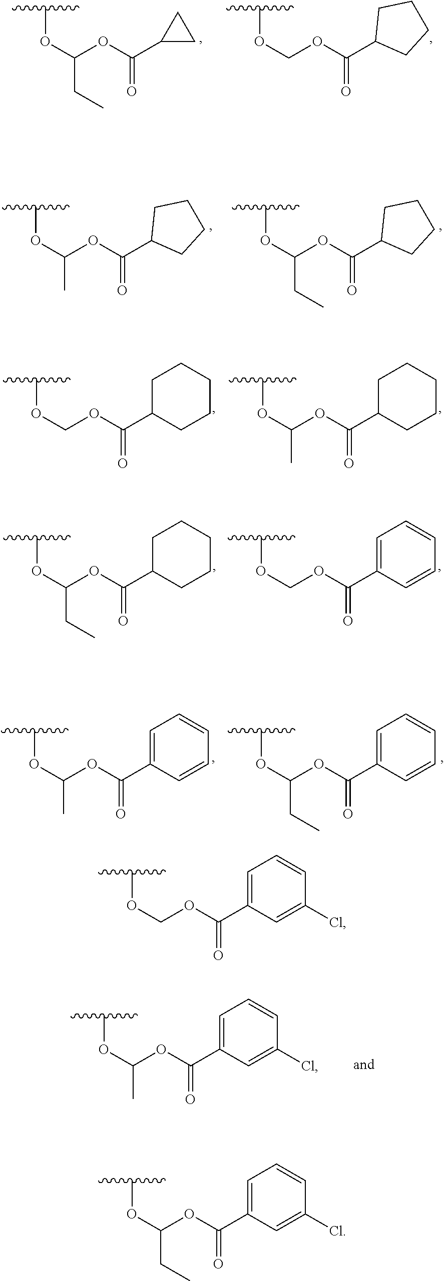

When R₂ is —OCH(Rₘ)OP(O)(ORₙ)₂, it may be selected from:

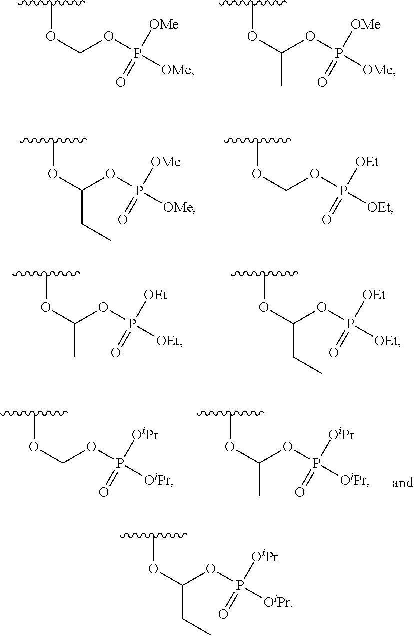

In some preferred embodiments, R₂ and R′₂ together form oxo, or R′₂ is H and R₂ is selected from hydroxy, and —OC(O)R^{A}, where R^{A} is as defined herein. More preferably, R₂ and R′₂ together form oxo, or R′₂ is H and R₂ is hydroxy. Most preferably, R₂ and R′₂ together form oxo.

When R₂ is

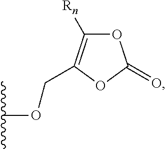

Rₙ is preferably selected from methyl, ethyl and isopropyl.

R₃ is preferably —H, methyl or ethyl, and more preferably is —H or methyl. Most preferably, R₃ is —H.

A is a ring which may be C₆ aryl or C₆ heteroaryl. Ring A is selected from:

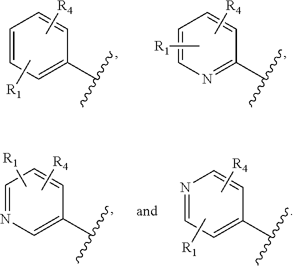

Preferably, ring A is selected from phenyl and pyridin-2-yl.

Ring A may be substituted with R₁ and R₄ at any chemically feasible ring atom. Preferably, when ring A is pyridin-2-yl, pyridin-3-yl, or pyridin-4-yl, R₁ and R₄ are each bonded to a carbon ring atom.

R₁ and R₄ may independently be at the ortho, meta or para position of ring A. By “ortho”, it is meant at the 2-position relative to the carbon atom bonded to R₂ and R₂′. This is shown in Formula (I^{ortho}) below, where both R₁ and R₂ are at an ortho position in ring A that is phenyl. By “meta” and “para” it is meant the 3-position and 4-positions, respectively, relative to the carbon atom bonded to R₂ and R₂′.

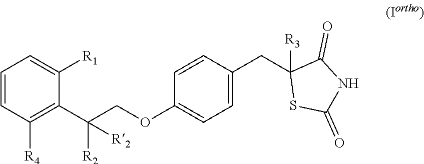

In some preferred embodiments, R₁ is in the meta or para position (3-position or 4-position relative to the carbon bonded to the R₂ and R′₂ groups). In these embodiments, R₄ is preferably hydrogen and can be bonded to a ring atom at any position that R₁ is not bonded to. Preferably, in these embodiments ring A is phenyl or pyridin-2-yl.

Preferably, when ring A is phenyl R₁ is in the ortho or meta position, more preferably in the meta position.

In these embodiments R₄ may be —H that is bonded to any remaining ring position.

In some embodiments the compound of Formula (I) is a compound of Formula (Ia).

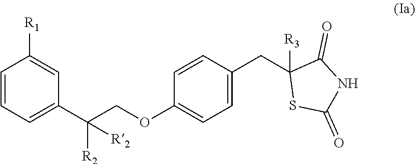

where R₁, R₂, R′₂ and R₃ are as defined herein.

In some embodiments ring A is phenyl and R₁ is selected from fluoro, chloro, bromo, methyl, ethyl, methoxy, ethoxy, —OCHF₂ and —OCF₃, more preferably R₁ is selected from methoxy and ethoxy. In these embodiments R₄ may be hydrogen.

Preferably, when ring A is pyridin-2-yl R₁ is in the 5-position relative to the nitrogen atom of the pyridyl ring. This position may also be referred to as the para position (4-position) relative to the carbon atom bonded to R₂ and R₂′. In these embodiments R₄ may be —H that is bonded to any remaining ring position.

In some embodiments the compound of Formula (I) is a compound of Formula (Ib).

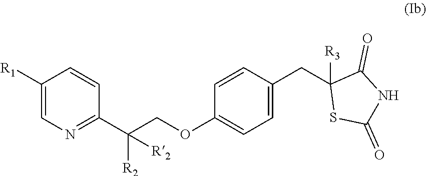

where R₁, R₂, R′₂ and R₃ are as defined herein.

In some embodiments ring A is pyridin-2-yl and R₁ is selected from fluoro, chloro, bromo, methyl, ethyl, methoxy, ethoxy, —OCHF₂ and —OCF₃, more preferably R₁ is selected from methyl and ethyl. In these embodiments R₄ may be hydrogen.

In some preferred embodiments, the compound of Formula (I) is selected from a compound of Formula (Ia) and a compound of Formula (Ib).

Preferably, R₂ and R′₂ together form an oxo group. In these embodiments the thiazolidedione compound may be a compound of Formula (11) or a pharmaceutically acceptable salt thereof:

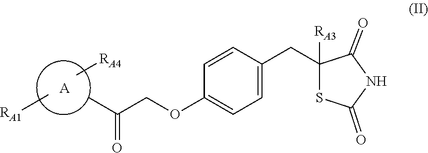

wherein each of R_{A1} and R_{A4} is independently selected from —H, halo, C₁₋₆ alkyl, and C₁₋₆ alkoxy, wherein the C₁₋₆ alkyl and C₁₋₆ alkoxy are optionally substituted with 1-3 of halo;
R_{A3} is —H or optionally substituted C₁₋₃ alkyl; and
   A is a phenyl, pyridin-2-yl, pyridin-3-yl, or pyridin-4-yl.

Preference for each of R_{A1}, R_{A3} and R_{A4} are as described herein for R₁, R₃ and R₄, respectively.

In further preferred embodiments, ring A is phenyl. In these embodiments the compound of Formula (I) or a pharmaceutically acceptable salt thereof may be a compound of Formula (IIa) or a pharmaceutically acceptable salt thereof:

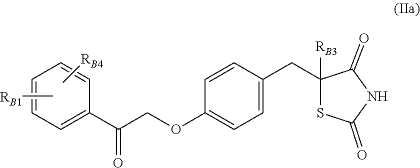

wherein each of R_{B1} and R_{B4} is independently selected from —H, halo, C₁₋₆ alkyl, and C₁₋₆ alkoxy, wherein the C₁₋₆ alkyl and C₁₋₆ alkoxy are optionally substituted with 1-3 of halo; and
R₃ is —H or optionally substituted C₁₋₃ alkyl.

Preference for each of R_{B1}, R_{B3} and R_{B4} are as described herein for R₁, R₃ and R₄, respectively.

Preferably, R_{B1} is C₁₋₆ alkyl or C₁₋₆ alkoxy, including methyl, ethyl, methoxy and ethoxy, in particular ethyl and methoxy. More preferably R_{B1} is C₁₋₆ alkoxy, more preferably methoxy or ethoxy, most preferably methoxy.

Preferably, R_{B3} and R_{B4} are each independently —H.

In some embodiments the compound of Formula (I) is a compound of Formula (IIa′):

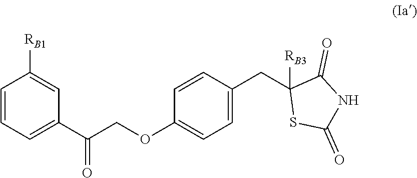

wherein R_{B1} is selected from —H, halo, C₁₋₆ alkyl, and C₁₋₆ alkoxy, wherein the C₁₋₆ alkyl and C₁₋₆ alkoxy are optionally substituted with 1-3 of halo; and
R₃ is —H or optionally substituted C₁₋₃ alkyl.

Preference for R_{B1} and R_{B3} are as described for compounds of Formula (IIa).

In other preferred embodiments, ring A is pyridin-2-yl. In these embodiments the compound of Formula (I) or a pharmaceutically acceptable salt thereof is preferably a compound of Formula (IIb) or a pharmaceutically acceptable salt thereof:

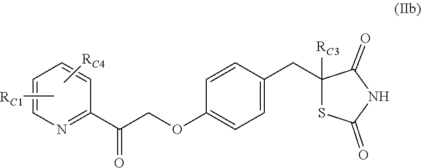

wherein each of R_{C1} and R_{C4} is independently selected from —H, halo, C₁₋₆ alkyl, and C₁₋₆ alkoxy, wherein the C₁₋₆ alkyl and C₁₋₆ alkoxy are optionally substituted with 1-3 of halo; and
R_{C3} is —H or optionally substituted C₁₋₃ alkyl.

Preference for each of R_{C1}, R_{C3} and R_{C4} are as described herein for R₁, R₃ and R₄, respectively.

Preferably, R_{C1} is C₁₋₆ alkyl or C₁₋₆ alkoxy, including methyl, ethyl, methoxy and ethoxy, in particular ethyl and methoxy. More preferably R_{C1} is C₁₋₆ alkyl, more preferably methyl or ethyl, most preferably ethyl.

Preferably, R_{C3} and R_{C4} are each independently H.

In some embodiments the compound of Formula (I) is a compound of Formula (IIb′):

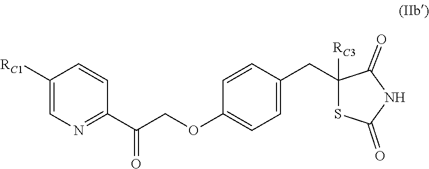

where R_{C1} is selected from —H, halo, C₁₋₆ alkyl, and C₁₋₆ alkoxy, wherein the C₁₋₆ alkyl and C₁₋₆ alkoxy are optionally substituted with 1-3 of halo; and
R_{C3} is —H or optionally substituted C₁₋₃ alkyl.

Preference for R_{C1} and R_{C3} are as described for compounds of Formula (IIb).

In preferred embodiments, the compound of Formula (I) is a compound of Formula (IIa) or (Ilb). More preferably, the compound is of Formula (IIa′) or (IIb′).

Most preferably, the compound of Formula (I) is selected from Compound A and Compound B, or a pharmaceutically acceptable salt thereof:

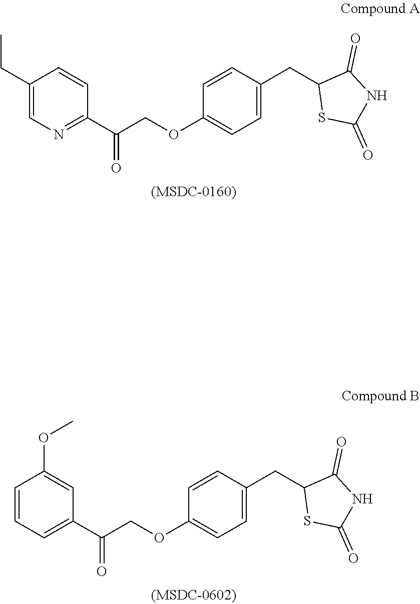 [SMILES: CCc1ccc(C(=O)COc2ccc(CC3SC(=O)NC3=O)cc2)nc1 ; COc1cccc(C(=O)COc2ccc(CC3SC(=O)NC3=O)cc2)c1]

A thiazolidinedione compound as described herein may be a salt of a compound of Formula (I), (Ia), (Ib), (II), (IIa), (IIa′), (IIb) or (IIb′), such as a sodium, lithium, potassium, calcium or magnesium salt. Of these, sodium and potassium salts are preferred, and sodium salts are most preferred.

Preferred compounds include the sodium and potassium salts of Compound A and Compound B, such as the sodium salt of Compound A or the potassium salt of Compound B.

The MPC inhibitor inhibits pyruvate transport into mammalian mitochondria, and could simply designated a mitochondrial pyruvate transport inhibitor, without reliance on any particular mechanism. However, wishing to be bound by theory, it is believed that this activity is mediated via the mitochondrial pyruvate carrier (MPC). The mammalian MPC is a complex (likely a heterodimer) composed of two protein components designated mitochondrial pyruvate carrier 1 (MPC1) and mitochondrial pyruvate carrier 2 (MPC2). The MPC inhibitor is believed to exert its inhibitory effect by binding to the MPC complex.

A compound may be tested for an inhibitory activity on mammalian mitochondrial pyruvate transport, or for direct inhibition of the mammalian (e.g. human) MPC by any appropriate means. For example, a suitable assay may measure pyruvate transport into isolated mitochondria, e.g. using an assay system as described by Herzig et al., Science (2012) vol. 337, issue 6090, p. 93-96. Alternatively, a pyruvate transport assay may employ recombinantly expressed mammalian (e.g. human) MPC1 and MPC2 components reconstituted into liposomes, as described for the corresponding yeast system by Tavoulari et al., EMBO J. (2019) 38: e100785. In either system, pyruvate transport may be measured in the presence and absence of the test compound to determine the test compound's activity.

The MPC inhibitor is not a PPAR-gamma agonist, or has lower PPAR-gamma agonist activity than pioglitazone and/or rosiglitazone. Preferably it has at least a 10-fold reduced potency as compared to pioglitazone and less than 50% of the full activation produced by rosiglitazone, e.g. in assays conducted in vitro for transactivation of the PPAR-gamma receptor. Suitable assays are described, for example, in WO 2012/149083, at Example 13.

Such assays may be conducted by first evaluation of the direct interactions of the molecules with the ligand binding domain of PPAR-gamma, e.g. using a commercial interaction kit that measures the direct interaction by fluorescence using rosiglitazone as a positive control, e.g. by a TR-FRET competitive binding assay. This assay may employ a terbium-labeled anti-GST antibody to label the GST tagged human PPAR-gamma ligand binding domain (LBD). A fluorescent small molecule pan-PPAR ligand tracer binds to the LBD causing energy transfer from the antibody to the ligand resulting in a high TR-FRET ratio. Competition binding by PPAR-gamma ligands displace the tracer from the LBD causing a lower FRET signal between the antibody and tracer. The TR-FRET ratio may be determined by reading the fluorescence emission at 490 and 520 nm.

PPAR-gamma activation in intact cells may be measured using a cell reporter assay, e.g. using the human PPAR-gamma ligand binding domain (LBD) fused to the GAL4 DNA binding domain (DBD) stably transfected into HEK 293H cells containing a stably expressed beta-lactamase reporter gene under the control of an upstream activator sequence. When a PPAR-gamma agonist binds to the LBD of the GAL4/PPAR fusion protein, the protein binds to the upstream activator sequence activating the expression of beta-lactamase. Following incubation with the agonists the cells are loaded with a FRET substrate and fluorescence emission FRET ratios are obtained at 460 and 530 nm after a suitable incubation time.

Preferably the MPC inhibitor has no, or substantially no, PPAR-gamma agonist activity.

For the avoidance of doubt, the MPC inhibitor is not pioglitazone or rosiglitazone.

### Pharmaceutical Compositions

The active agents described, i.e. the MPC inhibitor and the BCR-ABL inhibitor, will typically be formulated as pharmaceutical compositions, each independently for administration to a subject by a suitable route. As with all aspects of the invention, it is to be understood that reference to any given compound encompasses reference to a pharmaceutically acceptable salt thereof.

Examples of pharmaceutically acceptable salts are discussed in Berge et al., 1977, “Pharmaceutically Acceptable Salts,” J. Pharm. Sci., Vol. 66, pp. 1-19.

For example, if the compound is anionic, or has a functional group which may be anionic (e.g., —COOH may be —COO⁻), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al⁺³. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

If the compound is cationic, or has a functional group which may be cationic (e.g., —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous.

Examples of suitable organic anions include, but are not limited to, those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, glucheptonic, gluconic, glutamic, glycolic, hydroxymaleic, hydroxynaphthalene carboxylic, isethionic, lactic, lactobionic, lauric, maleic, malic, methanesulfonic (mesylate), mucic, oleic, oxalic, palmitic, pamoic, pantothenic, phenylacetic, phenylsulfonic, propionic, pyruvic, salicylic, stearic, succinic, sulfanilic, tartaric, toluenesulfonic, and valeric. Examples of suitable polymeric organic anions include, but are not limited to, those derived from the following polymeric acids: tannic acid, carboxymethyl cellulose.

The active agents may be formulated together in the same composition, or in separate compositions. Where they are provided in separate compositions, they may be administered to a subject simultaneously or substantially simultaneously, e.g. within 1 hour of each other. Alternatively they may be administered at different times, e.g. spaced by at least 1 hour, at least 6 hours, at least 12 hours, or at least 24 hours. Still further alternatively, they may be administered according to different administration regimes.

A pharmaceutical composition typically comprises a therapeutically effective amount of the relevant active agent, together with a pharmaceutically acceptable carrier, excipient or vehicle. A “therapeutically effective amount” is typically one sufficient to show benefit to the individual. The actual amount administered, and rate and time-course of administration, will depend on the nature and severity of the condition being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners.

The term “pharmaceutically acceptable carrier” includes any of the standard pharmaceutical carriers. Pharmaceutically acceptable carriers for therapeutic use are well known in the pharmaceutical art and are described, for example, in Remington's Pharmaceutical Sciences, 20th Edition, 2000, pub. Lippincott, Williams & Wilkins. For example, sterile saline and phosphate-buffered saline at slightly acidic or physiological pH may be used. Suitable pH-buffering agents may, e.g., be phosphate, citrate, acetate, tris(hydroxymethyl)aminomethane (TRIS), N-tris(hydroxymethyl)methyl-3-aminopropanesulfonic acid (TAPS), ammonium bicarbonate, diethanolamine, histidine, arginine, lysine or acetate (e.g. as sodium acetate), or mixtures thereof. The term further encompasses any carrier agents listed in the US Pharmacopeia for use in animals, including humans.

A pharmaceutical composition of the invention may be in unit dosage form. In such form, the composition is divided into unit doses containing appropriate quantities of the active component or components. The unit dosage form may be presented as a packaged preparation, the package containing discrete quantities of the preparation, for example, packaged tablets, capsules or powders in vials or ampoules.

The unit dosage form may also be, e.g., a capsule, cachet or tablet in itself, or it may be an appropriate number of any of these packaged forms. A unit dosage form may also be provided in single-dose injectable form, for example in the form of a pen device containing a liquid-phase (typically aqueous) composition.

The compositions may independently be formulated for any suitable route and means of administration, e.g. oral, intravitreal, rectal, vaginal, nasal, topical, enteral or parenteral (including subcutaneous (sc), intramuscular (im), intravenous (iv), intradermal and transdermal) administration or administration by inhalation. The formulations may conveniently be presented in unit dosage form and may be prepared by any of the methods well known in the art of pharmaceutical formulation.

Oral administration may be particularly preferred, e.g. in tablet formulations, since the MPC inhibitors and BCR-ABL inhibitors described in this specification generally display good bioavailability when administered by oral means.

The subject to be treated may be any animal or human. The subject is preferably mammalian, more preferably human. The subject may be a non-human mammal, but is more preferably human. The subject may be male or female.

### Definitions

An “alkyl” group refers to a saturated aliphatic hydrocarbon group containing 1-12 (e.g., 1-8, 1-6, or 1-4) carbon atoms. An alkyl group can be straight or branched. Examples of alkyl groups include, but are not limited to, methyl, ethyl, propyl, iso-propyl, iso-butyl, sec-butyl, tert-butyl, n-pentyl, n-heptyl, and 2-ethylhexyl. An alkyl group can be substituted (i.e. is optionally substituted) with one or more substituents such as hydroxy; halo, such as fluoro, chloro or bromo; carboxy (COOH); acyl, aroyl or heteroaroyl. A substituent may be bonded to a terminal carbon atom in the alkyl group, or a carbon atom that is away from a terminus of the alkyl group (i.e. a non-terminal carbon).

A “cycloalkyl” group refers to a saturated carbocyclic mono- or bicyclic (fused or bridged) ring of 3-8 (e.g. 5-8) carbon atoms. Examples of cycloalkyl groups include cyclopropyl, cyclobutyl, cyclopentyl, cyclohexyl, cycloheptyl, adamantyl, norbornyl, cubyl, octahydro-indenyl, decahydro-naphthyl, bicyclo[3.2.1]octyl, bicyclo[2.2.2]octyl, bicyclo[3.3.1]nonyl, bicyclo[3.3.2.]decyl, bicyclo[2.2.2]octyl, adamantyl, and ((aminocarbonyl)cycloalkyl)cycloalkyl. A cycloalkyl group may be substituted with one or more substituents as described herein.

An “aryl” group is a monocyclic, bicyclic or tricyclic ring systems in which the monocyclic ring system is aromatic or at least one of the rings in a bicyclic or tricyclic ring system is aromatic. Examples of an aryl group include phenyl and naphthyl. An aryl group may be optionally substituted with one or more substituents as described herein. Examples of a substituted aryl group include haloaryl, carboxyaryl, and hydroxyaryl.

A “heteroaryl” group refers to a monocyclic, bicyclic, or tricyclic ring system having 5 to 12 ring atoms wherein one or more of the ring atoms is a heteroatom (e.g., N, O, S, or combinations thereof) and in which the monocyclic ring system is aromatic or at least one of the rings in the bicyclic or tricyclic ring systems is aromatic. A heteroaryl group includes a benzofused ring system having 2 to 3 rings. For example, a benzofused group includes benzo fused with one or two 4 to 8 membered heterocycloaliphatic moieties (e.g., indolizyl, indolyl, isoindolyl, 3H-indolyl, indolinyl, benzo[b]furyl, benzo[b]thiophenyl, quinolinyl, or isoquinolinyl). Some examples of heteroaryl are azetidinyl, pyridyl, 1H-indazolyl, furyl, pyrrolyl, thienyl, thiazolyl, oxazolyl, imidazolyl, tetrazolyl, benzofuryl, isoquinolinyl, benzthiazolyl, xanthene, thioxanthene, phenothiazine, dihydroindole, benzo[1,3]dioxole, benzo[b]furyl, benzo[b]thiophenyl, indazolyl, benzimidazolyl, benzthiazolyl, puryl, cinnolyl, quinolyl, quinazolyl,cinnolyl, phthalazyl, quinazolyl, quinoxalyl, isoquinolyl, 4H-quinolizyl, benzo-1,2,5-thiadiazolyl, or 1,8-naphthyridyl. Monocyclic heteroaryls include furyl, thiophenyl, 2H-pyrrolyl, pyrrolyl, oxazolyl, thiazolyl, imidazolyl, pyrazolyl, isoxazolyl, isothiazolyl, 1,3,4-thiadiazolyl, 2H-pyranyl, 4-H-pranyl, pyridyl, pyridazyl, pyrimidyl, pyrazolyl, pyrazyl, or 1,3,5-triazyl. Monocyclic heteroaryls are numbered according to standard chemical nomenclature. Bicyclic heteroaryls include indolizyl, indolyl, isoindolyl, 3H-indolyl, indolinyl, benzo[b]furyl, benzo[b]thiophenyl, quinolinyl, isoquinolinyl, indolizyl, isoindolyl, indolyl, benzo[b]furyl, bexo[b]thiophenyl, indazolyl, benzimidazyl, benzthiazolyl, purinyl, 4H-quinolizyl, quinolyl, isoquinolyl, cinnolyl, phthalazyl, quinazolyl, quinoxalyl, 1,8-naphthyridyl, or pteridyl. Bicyclic heteroaryls are numbered according to standard chemical nomenclature. Examples of a substituted heteroaryl group include haloheteroaryl, carboxyheteroaryl, and hydroxyheteroaryl.

An “alkoxy” group refers to an —O-alkyl group where “alkyl” is as defined herein. Examples include —O-methyl (methoxy), —O-ethyl (ethoxy), —O-iso-propyl, —O-n-propyl, —O-tert-butyl, —O-n-butyl, —O-pentyl, and —O-hexyl.

A “hydroxyl” or “hydroxy” group refers to an —OH moiety.

A “halo” group refers to —F, —Cl, —Br or —I.

An “oxo” group refers to ═O.

### EXAMPLES

Metabolism is Highly Deregulated in CML LSCs with a Bias Towards Glucose Oxidation

To assess whether deregulation in metabolic pathway gene expression occurs in primitive CML cells and translates into differences in metabolite levels, requires analysis of transcriptomic (28, 29) and liquid chromatography-mass spectrometry (LC-MS) metabolomic datasets (12), comparing patient-derived CML cells to normal human stem/progenitor cells (FIG. 1a and Table 1). The LSC population in CML can be reproducibly characterised using the same cell surface expression markers as HSCs (30). Interrogation of a transcriptomic dataset (E-MTAB-2581) (29) generated from samples enriched for CML LSCs (CD34⁺CD38⁻) and normal HSCs (CD34⁺CD38⁻), using differential gene expression analysis, revealed an upregulation of genes involved in energy metabolism, including those belonging to fatty acid metabolism (CD36), one carbon metabolism (MTHFD1L) and amino acid uptake (SLC1A5 and SLC7A5) (FIG. 1b).

Gene Set Enrichment Analysis (GSEA) using general Reactome classifiers revealed that metabolism and metabolism-related processes were among the significantly enriched pathways (FIG. 1c). We subsequently examined the ontology of significantly upregulated genes using Panther (31). Using three different ontology datasets (Molecular function; Biological process; Protein class), significant increases were noted in the fraction of upregulated genes that belong to Catalytic activity, Metabolic process and Metabolite interconversion ontology categories in CMVL LSC, compared to all genes expressed in either HSCs or LSCs (data not shown). We next conducted GSEA on all genes using the Hallmark gene sets. In addition to MYC, p53 and E2F pathways that have previously been identified as dysregulated (29), multiple metabolic pathways display enrichment with the only negatively regulated pathways being KRAS signalling-down regulated genes and Bile acid metabolism (FIG. 1d). Upregulated pathways include central carbon metabolism pathways: glycolysis, oxidative phosphorylation, and fatty acid metabolism (FIG. 1e). These results agree with previous studies that suggest functional importance of these pathways in myeloid leukaemias (4, 5, 12, 32).

TABLE 1
Patient samples used in this study

| | | Age at | |
|---|---|---|---|
| Sample | Gender | Diagnosis | Clinical information |
| CML#1 | Male | 58 | ELN warning in CP on imatinib. |
| CML#2 | Male | 39 | ELN failure Nilotinib at diagnosis -> dasatinib. |
| CML#3 | Male | 61 | ELN failure. |
| CML#4 | Male | 46 | ELN failure Imatinib->nilotinib->dasatinib->SCT. |
| CML#5 | Male | 56 | ELN failure: BCR-ABL 0.11% at 12 months, |
| | | | MMR by 18 months: not resistant. |
| CML#6 | Female | 63 | Failed imatinib -> dasatinib. |
| CML#7 | Male | 61 | Optimal response to imatinib: BCR-ABL |
| | | | 0.04% at 12 months. |
| CML#8 | Male | 61 | Initial optimal response to imatinib, loss of |
| | | | response -> dasatinib; mutation screen |
| | | | negative; in MMR; optimal response. |
| CML#9 | Female | 35 | Optimal response to imatinib (MR4). |
| CML#10 | Female | 60 | Suboptimal response to imatinib 400 mg, |
| | | | increased to 600 mg, then reduced to |
| | | | 400 mg, optimal response: BCR-ABL 0.01%. |
| CML#11 | Male | 47 | ELN warning on dasatinib. |
| CML#12 | Male | 39 | Failed imatinib (compliance issues). |
| CML#13 | Female | 27 | ELN warning. |
| CML#14 | Male | 70 | Failed imatinib -> dasatinib, died. |
| CML#15 | Male | 70 | No TKI, no molecular monitoring; allogeneic |
| | | | SCT. Cured. |
| CML#16 | Male | 47 | Imatinib 400 mg; only achieved MR2, |
| | | | treatment failure, continued imatinib due to |
| | | | co-morbidities. |
| CML#17 | Male | 24 | Optimal response to imatinib at 3 months: |
| | | | BCR-ABL 0.07%, -> dasatinib due to side effects. |
| CML#18 | Female | 40 | ELN warning. |
| CML#19 | Male | 55 | Non-optimal response to imatinib 400 mg, |
| | | | therapy interrupted due to neutropenia |
| | | | intermittently -> dasatinib 100 mg daily: |
| | | | BCR-ABL PCR <0.87% at 6 weeks. |
| CML#20 | Female | 28 | ELN warning, responded to imatinib, |
| | | | achieved MMR on dasatinib |
| CML#21 | Male | 43 | |
| CML#22 | Female | 27 | ELN warning, non-optimal response to |
| | | | imatinib, achieved MMR on nilotinib |
| CML#23 | Male | 64 | Optimal response to imatinib, MMR at 6 months |
| CML#24 | Male | 33 | Achieved MMR on imatinib |
| CML#25 | Female | 31 | |

-> refers to next treatment; ELN: European LeukaemiaNet (recommendations for the management of CML); E: Extended Data Figure; CP: chronic phase; SCT: stem cell transplantation; MMR: major molecular response; MR4: BCR-ABL ≤0.01% international scale (IS); MR2: BCR-ABL <1% IS. All samples were >95% positive for BCR-ABL by FISH.

Steady state metabolite analysis from normal and patient-derived samples showed that malate was one of the metabolites with the largest fold change in CML (data not shown), in line with the deregulation of the TCA cycle and oxidative phosphorylation observed at the transcriptional level. To accurately assess metabolic activity and specific substrate contribution, we employed uniformly labelled ¹³C-tracing analysis of key carbon sources: glucose, palmitate, and glutamine (FIG. 1f). Analysis of ¹³C₆ glucose labelled samples revealed that for all metabolites labelled from glucose, the fraction of labelling was consistently higher in CML CD34⁺ cells (FIG. 1g) (12). While glutamine catabolism was increased in CML CD34⁺ cells compared to normal CD34⁺ cells, the difference was not as pronounced as for glucose (lower fold changes; FIG. 1h). Palmitate contributed fraction of labelling to a slightly higher extent in CML CD34⁺ cells but this did not reach statistical significance (FIG. 1i). However, given that the steady state levels of these metabolites are higher in CML cells than normal, increased fatty acid uptake via CD36 and subsequent oxidation may be needed to sustain these higher levels. Finally, we overlaid fraction labelling from glucose and glutamine with transcriptomics data (E-MTAB-2581) for joint-pathway analysis. This underlines that CML has a marked increase in central carbon metabolism both at the transcript and metabolite level (FIG. 1j). Notably, the increases fraction of labelling was more pronounced from glucose compared to glutamine. Specifically, there was an upregulation of genes involved in glucose uptake (SLC2A1), glycolytic enzyme transcripts and metabolites, as well as TCA cycle enzyme transcripts and metabolites. There was no upregulation in genes encoding enzymes involved in glutaminolysis (GLS and GLUD) but there was an upregulation of glutamine transporters (SLC7A5 and SLC1A5). These findings agree with transcriptomic analyses of murine and human CML studies (33-36). While CD34+ enrich for progenitors and stem cells, CD34⁺CD38⁻ enriches further for primitive stem cells. Thus, we compared the fate of ¹³C-glucose in these two cell populations in normal and CML samples (FIG. 1(k). While there was only modest amount of labelling in normal CD34⁺CD38⁻ and CD34⁺CD38⁺ cells, the labelling was significantly increased in both CML primitive populations. Notably there was only a slight, non-significant difference between CML CD34⁺CD38⁻ and CML CD34⁺CD38⁺ cells. Taken together these results demonstrate that CML LSCs are more metabolically active than their normal counterparts and favour glucose to fuel this upregulation.

### Imatinib Partially Reverses BCR-ABL Driven Reprogramming of CML LSC Transcriptome and Metabolome

As CML LSCs are more metabolically active than normal HSCs, we hypothesized that at least some of this activity was mediated by BCR-ABL kinase activity and would therefore be reversed following TKI treatment. To test this, we generated paired transcriptomics and metabolomic datasets from four CML CD34⁺ patient samples following culture in the physiological medium Plasmax supplemented as specified in Table 2 (FIG. 2a) (37).

TABLE 2
Supplements added for medium formulation used in this study.

| | | Catalogue | Final | |
|---|---|---|---|---|
| Supplement | Supplier | number | Concentration | |
| Albumax II | Thermofisher | 11021037 | 1 | mg/mL |
| | Scientific | | | |
| Human insulin | Merck | 19278-5ML | 10 | μg/mL |
| Glutamine | Thermofisher | 25030-024 | 0.65 | mM |
| | Scientific | | | |
| ¹³UC₅ Glutamine | Cambridge | CLM-1166-0.25 | 0.65 | mM |
| | Isotopes | | | |
| Glucose | Merck | G7021-100G | 5.5 | mM |
| ¹³UC₆ Glucose | Cambridge | CLM-1396 | 5.5 | mM |
| | Isotopes | | | |
| Sodium Palmitate | Merck | P9767 | 100 | uM |
| ¹³UC₁₆ Sodium | Cambridge | CLM-6059 | 100 | uM |
| Palmitate | Isotopes | | | |
| Transferrin | Merck | T4132-100MG | 7.5 | μg/mL |
| Beta | Merck | 21980 | 100 | μM |
| mercaptoethanol | | | | |
| Pyruvate | Merck | S8636-100ML | 100 | μM |
| P/S | Thermofisher | 15140122 | 1% | |
| | Scientific | | | |
| SCF | Peprotech | 3000-07 | 0.20 | ng/mL |
| G-CSF | Peprotech | 300-23 | 1 | ng/mL |
| GM-CSF | Peprotech | 300-03 | 0.20 | ng/mL |
| IL6 | Peprotech | 200-06 | 1 | ng/mL |
| MIP-a | Peprotech | 300-08 | 0.20 | ng/mL |
| LIF | Peprotech | 300-05 | 0.05 | ng/mL |

We applied the most commonly used TKI, imatinib, at a clinically relevant dose (2 μM) (38), which inhibited cell proliferation (inhibition of cell doubling time; p-value=0.0659) while inducing a modest, sample-dependent increase in the level of apoptosis (p-value=0.081) (data not shown) in agreement with previous studies (39). Differential gene expression revealed upregulation of 452 genes (2.8%) and downregulation of 576 genes (3.6%), including several genes that encode proteins that regulate metabolic pathways (data not shown). A significant increase in the negative regulator of cell cycle, p21 (CDKN1A) was observed, and downregulated genes included those involved in cell cycle progression CDK1 and TPX2, indicative of cell cycle arrest. At a protein level, albeit in different samples, the response in CDK1 levels varied in a sample-dependent manner, while phospho-CRKL, which is immediately downstream of BCR::ABL1 signalling, was decreased by imatinib in the two patient samples tested. Interestingly there was no change in CD36 which is highly upregulated in CMVL LSCs compared to normal HSCs. Furthermore, no changes were detected in glutaminase or any of the glutamine transporters. (Data not shown.) These results were cross-referenced with a patient dataset (E-MTAB-2594), obtained from CMVL CD34⁺ cells treated with imatinib for 7 days (40), where a higher proportion of expressed genes were significantly different (10,762: 56%) (data not shown). GSEA on generated transcriptomic data demonstrated that while immune response pathways, adipogenesis and p53 pathway were upregulated in response to imatinib (data not shown), the most downregulated pathways included oxidative phosphorylation, glycolysis, and fatty acid oxidation (FIG. 2b, d). Notably, similar results were obtained following longer (7 days) imatinib treatment (FIG. 2c, e), including consistent downregulation of oxidative phosphorylation in both datasets (FIG. 2d, e). Collectively, these data show that TKI-mediated BCR-ABL inhibition causes downregulation of key metabolic pathways, although not sufficiently to prevent LSC survival, suggesting that additional metabolism-targeting drugs may be required to effectively eradicate CML LSCs. However, the effect of imatinib on metabolism should be considered when adding metabolic inhibitors to imatinib treatment, with the preference to target deregulated pathways unaffected by imatinib or aiming to completely shut down pathways that are only partially inhibited by imatinib.

To obtain a global overview of metabolism during TKI treatment, we performed steady state analysis on imatinib-treated primary CML CD34+ cells, focusing on metabolites that can be reproducibly detected in primary samples (FIG. 2f). A significant increase in AIR with a corresponding trending (non-significant) decrease in AICAR likely reflects a decrease in purine synthesis related to decreased proliferation (data not shown). We observed a trend towards reduced ATP levels in imatinib-treated samples and a corresponding significant increase in AMP/ATP ratio in line with an induction of AMP-activated protein kinase (AMPK) (41,42) (FIG. 2g). We also detected decreases in oxidative stress and cellular redox (FIG. 2h, i) and TCA cycle metabolites (FIG. 2j). Mitochondrial respiratory capacity (measured by FCCP-induced oxygen consumption rate) was reduced in imatinib-treated samples. At a protein level, AMP-activated protein kinase (AMPK) and phospho-AMPK levels were variable depending on patient sample.

These variances may also be due to differences in time-point used for western blots compared to RNAseq (24 hours vs 48 hours). (Data not shown.) Notably, the more potent TKI nilotinib has been previously shown to increase phospho-AMPK in CD34+ CML cells (42). To examine pathways in an unsupervised manner, joint pathway analysis was conducted on data from paired RNA-seq and LC-MS samples. The most significantly affected pathways were pyruvate metabolism and the TCA cycle (FIG. 2k, l). Overall, the results of the integrated omic analysis are consistent with TKI treatment causing a reduction of the main CML central carbon metabolism pathways.

Imatinib Disrupts Nutrient Contribution to TCA Cycle and Redox Metabolites in CML CD34⁺ Cells without Affecting PC Activity

Given the reduction in central carbon metabolism observed following TKI treatment, we explored how CML cells maintain lower levels of metabolism while supporting survival. Using patient-derived CML CD34⁺ cells we examined the effect of imatinib on TCA cycle substrates: glucose, palmitate, and glutamine as well as on their contribution to the TCA cycle intermediates. To obtain a global overview of the relative substrate contribution to TCA cycle, and how this is altered by imatinib, the percentage of carbon pool from each tracer was merged to give the nutrient contribution plots of TCA cycle metabolites (FIG. 3a). Analysis using a two-way ANOVA revealed that palmitate and glutamine contribute a similar fraction of labelling to the TCA cycle metabolites except to citrate and glutamate, with palmitate contributing more to the former and glutamine more to the latter. For all TCA cycle-related metabolites, there was less contribution from glucose. However, this may be affected by the high palmitate concentration (100 μM) required for tracing experiments, resulting in an underestimation of glucose contribution. Indeed, the addition of 100 μM palmitate had a significant effect on the contribution of glucose, which can be seen in the comparison to the much higher contribution from glucose seen in medium where the only source of fatty acids is from lipid-rich Albumax II (data not shown).

Examining each dataset in detail, we found that glucose and glutamine contributions were reduced following imatinib treatment, while palmitate contribution was not significantly different (FIG. 3b-d). Decreases in glucose contribution were observed in TCA cycle metabolites (citrate, malate and aspartate), serine and glycine biosynthetic pathways (serine and glycine) and Cahill cycle (L-alanine). Decreases in labelling of TCA cycle metabolites and a decrease in the labelling of redox metabolites, glutathione, and glutathione disulfide (GSSG), was also observed in ¹³C₅ glutamine samples. Further analysis showed that for most metabolites, the decrease in glucose occurred to a greater extent than the decrease in glutamine fractional contribution (FIG. 3e). Notably, PC relative to PDH contribution of glucose was increased, measured by the ratio of m+3 (¹³C₃) to m+2 (¹³C₂) labelling in malate, citrate, and aspartate (FIG. 3f). This is due to a decrease in the PDH-derived fraction (m+2) without a decrease in the PC-derived (m+3) fraction (data not shown). These results are in agreement with paired transcriptomic data showing downregulation of PDH but no change in PC levels following imatinib treatment (FIG. 2I). Similar results were seen following nilotinib treatment, with reduction in total metabolite levels and ¹³C-glucose incorporation into TCA cycle metabolites, suggesting that the effect is mediated through inhibition of BCR::ABL1, but not other kinases affected by imatinib such as c-Kit (data not shown).

Changes in intracellular metabolism can be driven by changes in metabolite uptake and secretion. Thus, we quantified extracellular flux of spent medium samples from imatinib-treated and untreated primary CML CD34⁺ cells using a YSI bioanalyser. Analysis of these data showed that imatinib treatment decreased glutamine consumption (data not shown). While imatinib-treated samples consumed less glucose than control cells, when corrected for a decrease in proliferation this difference disappeared. It is noteworthy that the cell density used here was designed to maintain metabolic steady state and avoid depleting nutrients, so it is possible that differences in glucose consumption were below the assay detection limit.

Overall, these data suggest that PC-mediated glucose anaplerosis persists in imatinib-treated CML CD34⁺ cells and may contribute to residual TCA cycle activity. To investigate the relevance of these findings to the therapy resistant CD34⁺CD38⁻ population we quantified the effect of imatinib on ¹³C-glucose labelling in sorted cells (FIG. 3g). While imatinib caused a statistically significant reduction in glucose incorporation in CML CD34⁺CD38⁻ cells, the relative PC/PDH activity increased in all three patient samples in this primitive population with a trend towards statistical significance (p-value=0.0870).

Additionally, given that orally-bioavailable TKIs provide life-long disease control for the majority of CML patients, it is important to consider the toxicity of available inhibitors for fatty acid metabolism, the absence of anaplerosis from fatty acids, as well as the high levels of fatty acid oxidation observed in normal LSCs when prioritising targetable metabolic pathways. We therefore aimed to target glucose anaplerosis in CML LSCs.

### PC Activity can be Abrogated Using an MPC Inhibitor in CML CD34⁺ Cells

As it is possible that ¹³C₃-labelled TCA metabolites could be the product cytoplasmic maleic enzyme 1 (ME1) activity (43), we decided to use inhibitors of mitochondrial pyruvate carriers (MPC) to prevent pyruvate entry into mitochondria, allowing for distinction between cytosolic and mitochondrial metabolites (FIG. 4a). While the most commonly used research-grade MPC inhibitor is the tool compound UK-5099, MSDC-0160 is of relevance as it has successfully undergone Phase 2 clinical trial for diabetes (NCT00760578). We screened several concentrations of these compounds in the K562 CML cell line. This is critical as the albumin present in either FBS or in Albumax II, which is present in medium for primary sample culture, has been shown to bind each drug, reducing their bioavailability and necessitating higher concentrations for complete inhibition in cell culture compared to Seahorse assay that does not comprise FBS (44). While both compounds led to a concentration-dependent reduction in glucose contribution to fraction of labelling as previously reported (44), we observed the persistence of the m+3 fraction in aspartate and malate which indicates the presence of cytosolic ME1 activity, while there are reductions in all other fractions (data not shown). Furthermore, these results indicate that alpha-ketoglutarate (AKG) and AKG-derived glutamate are a more appropriate approximation of mitochondrial glucose oxidation for this cell line, with both inhibitors reducing levels of all labelled fractions in these metabolites in a concentration-dependent manner.

We next tested MSDC-0160 treatment on primary CML CD34⁺ samples. Steady state analysis only showed significant changes at a metabolic level at the highest dose of 100 μM (FIG. 4b). Unlike that noted in K562 cells, MSDC-0160 reduced both total glucose contribution and m+3 contribution to TCA cycle metabolites in a dose-dependent manner in primary CML CD34⁺ samples (FIG. 4c). Using 20 or 100 μM MSDC-0160 caused significant decreases to both m+2 and m+3 fractions (FIG. 4c) unlike imatinib which only affected m+2 fractions (not shown), with variable effects on TCA cycle metabolite m+3/m+2 ratios. While the dose required to fully inhibit labelling is relatively high (100 μM), this is consistent with high FBS binding of the drug as previously reported (44). This inhibition was also achieved in the presence of stromal cells. Moreover, we confirmed that inhibition of glucose oxidation was achieved in the primitive CD34⁺CD38⁻ population, to a similar extent as in the CD34⁺CD38⁺ progenitor population. (Data not shown.) Subsequently, we compared expression of PC from the E-MTAB-2581 dataset with independent studies that focused on primary CML progenitors/stem cells using either cell surface markers or quiescence as markers of primitive cells. These studies show an increase in PC levels in all datasets, including GSE15811 where the effect of BCR-ABL induction was modelled by exogenous expression in normal (cord blood) CD34⁺ cells (FIG. 4d). Finally, we examined the effect of PC expression across other cancers and found that high PC expression is associated with poorer survival in stomach adenocarcinoma, sarcoma, and ovarian cancer (data not shown).

Taken together these data demonstrate that glucose oxidative metabolism is distinct in primary CML CD34⁺ cells (C34⁺CD38⁻ cells) compared with both normal CD34+ cells (C34⁺CD38⁻ cells) and CML cell lines, and mitochondrial pyruvate metabolism can be inhibited with a clinically relevant MPC inhibitor.

### PC Ablation or Inhibition of Mitochondrial Pyruvate Import Sensitises CML Cells to Imatinib

To test the functional impact of PC inhibition, we generated a PC knockout K562 CML cell line using CRISPR-Cas9 technology (FIG. 5a). PC deletion did not affect proliferation of K562 cells (FIG. 5b). LC-MS was then applied to investigate if PC deletion reduced glucose contribution to TCA cycle metabolites. Using AKG and AKG-derived glutamate as readouts of mitochondrial pools, we observed a marked reduction in the m+5 fraction (FIG. 5c). This m+5 fraction is derived from the condensation of PC-m+3 and PDH-m+2 (45). The m+2 fraction was unchanged or slightly increased while m+3 fractions were slightly decreased, which is likely due to persistent cytoplasmic maleic enzyme activity (data not shown). To test the effect of TKI on PC deletion, control and knockout cell lines were treated with imatinib and cell viability measured. While PC ablation sensitised K562 to imatinib in both viability and colony-forming cell (CFC) assays, this was not observed using the FDA-approved protein translational inhibitor omacetaxine mepesuccinate (used on occasions for advanced phases of CML), suggesting selective increased reliance on glucose anaplerosis in TKI treated cells (FIG. 5d, e). Supporting this, knockout of PC in the KCL22 cell line, which have lower levels of PC, failed to increase sensitivity to imatinib (data not shown). As there is no clinically applicable PC inhibitor available and MPC inhibition should replicate its effect, we next tested the impact of combining MSDC-0160 with imatinib on colony formation potential of CML CD34⁺ cells isolated from six CML patients. While imatinib reduced the number of colonies, there was a further concentration-dependent reduction by the addition of MSDC-0160 (FIG. 5f). We then tested the effect of this combination on primary normal CD34⁺ cells to ensure that it is not toxic due to off-target effects. Encouragingly, no significant effect of single agent or combination treatment was observed on the CFC potential of these cells (FIG. 5g). The above data therefore support the rationale of combining a mitochondrial pyruvate transport inhibitor with imatinib in vivo.

Inhibition of MPC Synergises with Imatinib to Target Human CML LSCs In Vivo

To assess the clinical relevance of these findings we employed a patient-derived xenograft model, transplanting CML CD34⁺ cells into sub-lethally irradiated immunocompromised NRG-W41 mice (46) (FIG. 6a). Eight weeks post-transplant, mice were randomly assigned to groups for 4 weeks of treatment: vehicle, imatinib (50 mg/kg BID), MSDC-0160 (30 mg/kg/day) and combination of imatinib and MSDC-0160. All treatment arms were well tolerated, indicated by consistent body weight throughout the study (FIG. 6b). At the experiment end point, bone marrow cells were collected, and engraftment was determined by flow cytometry (data not shown). While there was a reduction in percentage of human CD45⁺ leukocytes and total number of bone marrow CD45⁺ cells in the combination group, this did not reach statistical significance (data not shown). There was also a relative and total reduction of CD34⁺ cells when comparing vehicle or imatinib arms to the combination, although this also did not reach statistical significance (FIG. 6c, d). However, when examining the most primitive CML population (CD34⁺CD38⁻), there was a marked reduction in both the percentage and absolute number of human CML LSCs following combination treatment (FIG. 6e, f). Thus, the combination of MPC inhibition with standard-of-care drug imatinib causes a significant reduction in therapy resistant CD34⁺CD38⁻ CML cells at clinically administrable doses in vivo. A corresponding experiment was performed using ABL-001 (asciminib) as an alternative to imatinib, illustrated in FIG. 7. Engraftment was performed for 12 weeks rather than 8. ABL-001 caused a variable increase in engraftment of human CML cells in bone marrow with CD34⁺CD34⁺ cells not affected. While the combination treatment did not reduce the total numbers of CML CD45⁺CD34⁺CD38⁻ cells (LSC) due to the effect of ABL-001 on engraftment, it did cause a reduction in the percentage of human CML LSCs both on its own and in combination with ABL-001, showing that similar effects to those observed with imatinib can also be obtained with other BCR-ABL kinase inhibitors.

### Discussion

Transformation from a normal cell to a cancerous one requires metabolic changes to fuel bioenergetic demands required for increased proliferation or adaptation to a hostile environment (47). Metabolic adaptation may also occur during anti-cancer treatment. For example, AML cells have been shown to exhibit transient metabolic adaptation in vivo, driving resistance to chemotherapy (5, 48). An important consideration is that rapidly proliferating normal cells also undergo metabolic reprogramming. For example, pyruvate metabolism has previously been shown to inhibit clonal expansion of normal clonogenic haematopoietic progenitor cells, which necessitates the inclusion of normal cells in metabolic studies.

In CML, the constitutively active BCR-ABL kinase drives proliferation of the leukaemic clone, through stimulation of several key cell survival pathways frequently activated in cancer. Recently, upregulation in key metabolic pathways such as central carbon metabolism has also been demonstrated in CML cells, including therapy resistant CML LSCs, which is required to facilitate and sustain CML LSC survival (12,36). However, the role or existence of these cytoprotective changes following acute or prolonged exposure to TKI treatment in clinically relevant cell population is not well understood and requires detailed metabolic analysis of primary human samples. Transcriptomic analyses revealed a significant deregulation of multiple metabolic pathways in chronic phase CML LSCs when compared with normal HSCs, suggesting BCR-ABL mediated metabolic reprogramming in the early phase of leukemogenesis. This was also apparent following analysis of stable isotope tracing of TCA cycle substrates. Specifically, when compared to normal cells, primitive CML cells have significantly higher glucose metabolism, slightly higher glutamine metabolism and only modest differences in fatty acid metabolism. Overlay of transcriptomic data (28, 29) with metabolomic data (generated here and previously (12)) confirmed that CML LSCs have increased central carbon metabolism at both transcript and metabolic levels. Specifically, there was an upregulation of glucose transporter (SLC2A1) and glycolytic enzyme transcripts. Interestingly, there was no upregulation of transcripts of glutaminolysis enzymes (GLS and GLUD) but there was an upregulation of glutamine transporters (SLC7A5 and SLC1A5). Glutamine transporter upregulation driven by c-MYC has been reported previously (49-51), but whether deregulated c-MYC (29) drives this upregulation in CML LSCs warrants further investigation.

While metabolic reprogramming offers multiple targets for therapeutic intervention, we focused on pathways that remain deregulated in the presence of imatinib, as its clinical use in CML is so ubiquitous it can be considered as a baseline. To model the effect of TKI treatment, we isolated and cultured patient derived CML CD34⁺ cells in physiological culture medium, in the absence or presence of imatinib-mediated BCR-ABL inhibition and subjected them to RNA-seq and LC-MS mediated metabolomics to generate paired multi-omics datasets. We subsequently used isotope assisted metabolomics and uniformly labelled ¹³C tracers of the key TCA cycle carbon sources glucose (¹³C₆), glutamine (¹³C₅) and palmitate (¹³C₁₆) and showed that the fractional contribution of pyruvate anaplerosis via PC and fatty acid oxidation are the only metabolic pathways not decreased (reverted) following imatinib treatment. While deregulated PC activity has been shown to be important in other malignancies (52, 53), its importance to leukaemia is unknown.

As mitochondrial and cytosolic metabolite pools can confound the interpretation of isotope tracing experiments, we utilised MPC inhibitors (UK-5099 and MSDC-0160 (25-27)) to confirm that pyruvate carboxylation occurs in the mitochondria. Additionally, we demonstrated that CRISPR-Cas9 mediated deletion of PC sensitises CML cells to imatinib. Leveraging this metabolic vulnerability, we combined MSDC-0160 with imatinib to target CML LSCs in vitro and in vivo. In conclusion, our data highlights a strong bias towards glucose metabolism in CML LSCs and a novel clinically relevant downstream target, mitochondrial pyruvate metabolism.

In terms of potential limitations and future work, while our experimental design aims to examine nutrient contribution in a physiological setting and shows that palmitate (100 μM) is a major contributor to TCA cycle, it is important to state that the non-assigned fraction is unknown. In addition, a wide range of human plasma free palmitate concentrations has been reported (HMDB: 25-1000 μM), even within the same study using different methods (66±9.9-122±48 μM) (54). The variability of reported palmitate levels, the potential effect from non-labelled palmitate from plastic, and the absence of other types of lipids means that FIG. 4a may not present an accurate reflection of relative nutrient importance. Furthermore, the precise mechanism by which PC is upregulated in CML LSCs and persists in the presence of TKI treatment is unclear. Finally, whether this TKI-escape mechanism exists in other malignancies warrants additional investigations.

In terms of clinical relevance, CML represents a paradigm for targeted therapy in cancer with the potential for cure. While the introduction of TKIs have revolutionised treatment of CML, a small but important minority of patients fail to respond (55). CML LSCs are inherently insensitive to TKI treatment, and it is within this cellular fraction that drug resistance and disease progression evolve. As CML is managed by orally-available TKI treatments in the majority of patients, testing medication that requires parenteral administration is not practical. MSDC-0160 has been shown to specifically inhibit MPC activity in a variety of cell types (27). It belongs to a class of thiazolidinediones (also called glitazones), and has already completed a promising Phase II trial for type 2 diabetes (NCT00760578; NCT01103414) (25) and for patients with Alzheimer's disease (NCT01374438) (26). Here we have shown that the clinically relevant and orally-available MPC inhibitor, MSDC-0160, could be both efficacious and practical to combine with TKI to improve treatment responses in CML patients.

### Materials and Methods

### Statistical Analyses

No statistical methods were used to determine sample size. For experiments, a minimum of four samples was used to give adequate power. The investigators were not blinded to sample or treatment during experiments.

### Reagents

Imatinib mesylate was purchased from LC Laboratories (1-5508). A stock solution of 10 mM was prepared in sterile Milli-Q water and stored at 4° C. MSDC-0160 (Apex Biotechnology: B3702) UK5099 (Merck: PZ0160-5MG) and Omacetaxine mepesuccinate (ChemGenex Pharmaceuticals) were made up in DMSO. Stock concentrations were 50 mM for MSDC-0160, and 10 mM for UK5099 and Omacetaxine. For in vivo experiments MSDC-0160 was made up weekly at 3 mg/ml in vehicle that consisted of 1% sodium carboxymethylcellulose (Sigma: Cat #C5678) and 0.01% Tween 80 (Sigma Cat #P4780) (stored at 4° C.). This forms a colloidal solution that was resuspended using magnetic stirrer prior to each dose.

Asciminib (ABL001) was purchased from MedChemExpress (Cat #HY-104010/CS-7655). ABL001 was made up weekly at 1 mg/ml in vehicle that consisted of 0.5% methylcellulose (Sigma: Cat #M7027) and 0.5% Tween 80 (Sigma Cat #P4780) (stored at 4° C.). This forms a colloidal solution that was resuspended using magnetic stirrer prior to each dose.

### Primary Samples and Ethical Approval

Primary CML samples were sourced from CML patients either from 50 mL peripheral blood or leukapheresis product. Patients were in chronic phase CML at the time of diagnosis and gave informed consent in accordance with the Declaration of Helsinki and approval of the National Health Service (NHS) Greater Glasgow and CLyde Institutional Review Board. The CD34⁺ cells were isolated using the CliniMACS (Miltenyi Biotec) and purity verified to be >95% by flow cytometry (Apoptosis and CD34 analysis). Non-leukemic cells were isolated from femoral head material using human CD34 MicroBeads (Miltenyi Biotec), according to the manufacturer's instructions. Purity was verified by flow cytometry to be >90%. Ethical approval has been granted to the research tissue bank (REC 15/WS/0077) and for use of surplus human tissue in research (REC 10/S0704/60).

### Cell Culture

Primary CML samples were thawed and recovered overnight in Plasmax, a physiological cell culture medium (37). This medium was supplemented with nutrients and growth factors as described in Table 2, then filter sterilised through a 0.2 μM filter (Fisher Scientific: 10509821). Cells were seeded at a density of 400,000 cells/mL, a density that was found to avoid nutrient depletion (data not shown).

For conjugation of palmitate, a 20 mM stock was made in 100% EtOH. This was incubated on a shaking heater, 60° C. until the solution clarified. The palmitate was then added to 10% BSA (ultra-fatty acid free in EBSS, Roche, 03117057001) to give a palmitate:BSA ratio of 1:3 BSA. This solution was left for 15 min in water bath (37° C.), and then added to final concentration in complete medium. For cell line experiments, the medium was RPMI with 10% dialysed FBS and 1% penicillin/streptomycin. For tracing experiments 11 mM ¹³C₆ glucose was added to glucose-free RPMI.

### Sample Preparation

Cells were counted using a CASY counter with the following parameters: E-cur 7.5-22.5, N-cur 5.25-20.5. Cell number was multiplied by peak volume (size) and this number was used to calculate volume of solvent to extract in. Cells were pelleted by centrifugation (400 RCF, 10 minutes, room temperature) at which medium was removed for YSI analysis. Cells were then washed twice with ice cold PBS (Calcium and Magnesium free: Thermo Fisher Scientific) with pulse centrifugation (12,000RCF, 15 seconds, 4° C.) used between and after washes. Cells were then extracted in LC-grade ACN:MeOH:H₂O solvent (−20° C., 5:3:2) by disrupting the pellet by pipetting followed by a 5 second vortex. Finally, debris was pelleted by centrifugation (16,000RCF, 10 minutes, 4° C.), supernatant transferred to LC-MS glass vials that were stored at −80° C. until analysis.

Doubling time over 48 hours was calculated by dividing the cell density into 4 times the seeding density (4×4×10\hat{ }5 cells/mL=16). For stromal co-culture experiment, irradiated M2-10B4 and S1/S1 mouse cell lines that are genetically-engineered to express human cytokines were seeded (8×10\hat{ }4 cells each in 1000 uL) in DMEM supplemented with 10% FBS and hydrocortisone onto collagen-coated plates. The following day, medium was removed and 2×10\hat{ }5 primary CML resuspended in 1000 μl of Plasmax (13-UC-glucose), seeded on top of the feeder cell layer for 24-hour culture in absence or presence of 50 uM MSDC-1060. Non-adherent cells were then harvested, and samples prepared for LC-MS.

### LC-MS

The LC system composed of a ZIC-pHILIC column (SeQuant, 150×2.1 mm, 5 μm, Merck KGaA) with a ZIC-pHILIC guard column (SeQuant, 20×2.1 mm) with an UltiMate 3000 HPLC system (Thermo Fisher Scientific). The aqueous mobile-phase solvent was 20 mM ammonium carbonate-0.1% ammonium hydroxide solution and with acetonitrile being used for organic mobile phase. A linear biphasic LC gradient was conducted from 80% organic to 80% aqueous for 15 min for a total run time of 22 min. The column temperature was maintained at 45° C. flow rate set to 200 μL/min.

The MS used in this study was a qExactive Plus Orbitrap Mass Spectrometer (Thermo Fisher Scientific) operating in polarity switching mode. The MS set up was calibrated using a custom CALMIX in both ionization modes before analysis and a tune file targeted towards the lower m/z range was used. Full scan (MS1) data was acquired in both ionization modes in profile mode at 70,000 resolution (at m/z range 75-1000), an automatic gain control (AGC) target of 1×10⁶ (max fill time of 250 ms), with spray voltages +4.5 kV (capillary +50V, tube: +70 kV, skimmer: +20V) and −3.5 kV (capillary −50V, tube: −70 kV, skimmer: −20V) and s-lens RF level of 50 for the front optics. The capillary temperature 375° C., probe temperature 50′C, sheath gas flow rate 25, auxiliary gas flow rate 15a.u. 15 and sweep gas flow rate of 1a.u.

The mass accuracy achieved for all metabolites was below 5 ppm. Data acquisition was achieved with Thermo Xcalibur 4.3.73.11 software.

### LC-MS Analysis

The peak areas of different metabolites were determined using Tracefinder 4.1 software (Thermo Fisher Scientific). Metabolites were identified by accurate mass of the singly charged ion and by known retention times on the pHILIC column. A commercially available standard compound mix (Merck: MSMLS-1EA) had been analysed previously on our LC-MS system to determine accurate ion masses and retention times. The 13C labelling was determined by quantifying peak areas for the accurate mass of all isotopologues of each metabolite.

### Steady State Analysis.

Two independent experiments were performed, data normalised to first sample in each experiment, processed through Autoplotter (56), samples as experiments, and normalised data as replicates. These data were processed on Metabonalyst using the R log transformation and mean-centring to ensure data followed a normal distribution. For MSDC-0160 treated samples a batch correction (to non-drug control) was used prior to analysis using Metabonalyst. Volcano plots were generated, and an FDR adjusted t-test threshold 0.1 was utilised. Note both fold changes and p-values are log transformed. Top-25 scoring metabolites are included in heatmap.

### Analysis of Stable Isotope Tracing Experiments

Biological and technical replicates were processed through Autoplotter (56) and natural abundance was corrected for. Fraction of enrichment was calculated as m+2 and above. For CML samples treated with or without imatinib, multiple paired T-tests were conducted on Log 10 transformed values in Graphpad Prism. The correction for multiple comparisons was the two-stage step-up method of Benjamini, Krieger and Yekutieli with an FDR (Q) value of 10%. For volcano plots Log 10 FC vs Log 10 (q value) are plotted with q values>5% in red and >10% in blue. For normal vs CML comparisons, the process was the same with the exception that unpaired t-tests were used. For PC activity in normal vs CML, this is denoted by fraction m+3. For imatinib vs untreated we look at relative activity to PDH. IE., the m+3/m+2 ratio and conducted multiple t-tests with correction for multiple comparisons being the two-stage step-up method of Benjamini, Krieger and Yekutieli with an FDR (Q) value of 10%. To examine contribution to carbon pool we corrected contribution of each isotopologue by the number of carbons it contributes to a given metabolite, i.e., for glutamine contribution from m+5 is greater than m+3. Note that for cell line experiments standard deviation is shown.

### Bioanalyser

A YSI 2900 biochemistry analyser was used as per the manufacturer's instructions to quantify glucose, lactate, glutamine, and glutamate in the media. The concentration of each metabolite was normalised to cell number and the rate of uptake or secretion per hour was calculated relative to cell free medium. The exchange rate per 48 hours [secretion (+) or consumption (−)] for a specific metabolite (x) was obtained according to the following equation:

$$\frac{Δ metabolite}{( cell number day 0 + cell number day 2 ) / 2}$$

where Δmetabolite=((x) mmol spent medium−(x) mmol cell-free medium).

### Multi-Omic Analysis

The joint pathway tool of Metabonalyst (57) was used for analysis of paired data. Here we used the hypergeometric test for enrichment analysis, the topology measure was degree centrality, and the integration method was combined queries.

### Gene Ontology Analysis

Panther (31), (version 17.0) was used for mapping to different sets and Fisher's exact test used to compare fraction of number of upregulated genes per pathway/total number of upregulated genes with same fraction of all expressed genes.

### RNA Extraction

RNA was extracted from 200,000 CML CD34+ cells using an RNA easy mini kit (Qiagen) according to the manufacturer's instructions.

### RNA-Seq

Libraries were prepared using the Illumina TruSeq Stranded mRNA LT Kit (Illumina) and run on the Illumina Next Seq 500 using the High Output 75 cycles kit (2×36 cycles, paired end reads, single index; Illumina). FastQ files were generated using Illumina's bcl2fastq (v. 2.20.0.422).

QC, alignment, and parsing of files into count matrices was performed in command line, with subsequent differential gene expression (DGE) analysis performed in R (1.2.1335). Adapter trimming was performed using Scythe (version 0.981), and Sickle (version 0.940), was used to trim bases with quality scores of less than 20. Prior to and after this pre-processing fastqc (version 0.11.2) was run to ascertain sequence quality, alongside the efficacy of the pre-processing steps. Trimmed reads were indexed and aligned using Hisat2 (version 2.1.0). Hisat2 indexes (GRCh38 genome_tran) were obtained from the John Hopkins Center for Computational Biology, 2020. Samtools (version 0.1.19044428cd) view was used to convert the resulting .sam to .bam files, whilst samtools sort was used to sort the .bam files. Assembly was achieved through the use of stringtie (John Hopkins Center for Computational Biology, 2020), with output .gtf files converted to count matrices using the python script prepDE.py (stringtie version 1.3.3b.Linux_x86_64). Reads were assembled using an annotated reference human genome (GRCh38.p13), obtained from GENCODE (GENECODE, 2020). DESeq2 (version 1.26.0) was used to generate results sets from the gene and transcript count matrices. G genes with read counts too low to allow for the calculation of p and adjusted p-values (padj: Benjamini-Hochberg) were removed from the data sets leaving gene and transcript counts of sizes 16,069 and 45,218 respectively.

### Microarray Analysis

Data were analysed using Limma (version 3.34.9).

### GSEA Analysis

GSEA (version 4.1) was conducted on pre-ranked lists (ranked by pi score calculated by multiplying LOG fold change by −LOG (corrected p-value)).

### Survival Analysis

Forest plot of Hazard ratios and Kaplan Meier plots, Log-rank p-values and 95% confidence intervals were calculated using Kaplan-Meier plotter or SurvivalGenie (AML). Cut-off points were automatically calculated within software.

### Western Blot Analysis

Cells were lysed in RIPA buffer containing mini-Complete protease inhibitor cocktail and phosphatase inhibitors (both Roche). Total protein concentration was quantified using a Pierce BCA kit (Thermo Fisher Scientific: 23227). Equal amounts of protein (5-7.5 μg) were heated at 95° C. for 5 min and separated (120V) in 4-12% gels (Novex) for SDS-PAGE. Proteins were transferred onto PDVF membranes (Thermo Fisher Scientific: 21882) then blocked in 2% BSA (in Tris-buffered saline, 0.01% Tween (TBS-T)) for one hour. Next, membranes were incubated overnight at 4° C. with the primary antibodies. The membranes were rinsed three times with TBS-T, then incubated with secondary HRP-linked antibodies (1:10,000) for 1 hour at room temperature. The SuperSignal West Femto Maxi detection system was used (Thermo Fisher Scientific: 34095) and imaging was carried out using a LI-COR Odyssey Fc gel-doc system.

### Apoptosis and CD34 Analysis

Cells were stained with Annexin V (fluorescein isothiocyanate (FITC, BioLegend: 640906, 5 uL/test), 7-AAD (BD Bioscience: 559925, 5 uL/test) and CD34+(APC, BD Bioscience: 555824, 2 uL/test) in 50 uL Hanks' Balanced Salt Solution (HBSS) for 20 minutes. CML cells were analysed by flow cytometry (BD FACSVerse) and data were analysed using Flo Jo (version 10).

### CR/SPR-Cas9 Mediated Deletion

To target the human PC gene, guides were designed using the optimized tool https://www.genscript.com/gRNA-database.html. Two guides were chosen and ordered from Integrated DNA Technologies. These were annealed and cloned in Bsmb I-digested lentiCRISPRv.2-puro (RRID: Addgene_52961). After stable integration of lentiCRISPRv.2 using lentiviral transfection and 1-week selection using puromycin (2.5 μg/ml), guides were validated by performing Western blotting. Oligonucleotides from IDT are as follows:

g1 forward:
CACCGCAGGCCGCGGCCGATGAGAT
g1 reverse:
AAACATCTCATCGGCCGCGGCCTGC
g3 forward:
CACCGACAGGTGTTCCCGTTGTCCC
g3 reverse:
AAACGGGACAACGGGAACACCTGTC

### Lentivirus Production

Lentiviruses for pLentiCRISPRv.2 were produced by the calcium phosphate method using pCMV-VSV-G (envelope plasmid: RRID: Addgene_8454) and psPAX2 (packaging constructs: RRID: Addgene_12260) vectors and human embryonic kidney (HEK) 293FT cells for transfection.

### Patient Derived Xenografts Experiments

For in vivo engraftment, 1 million CML CD34⁺ cells, were transplanted via tail vein into sublethally irradiated (2.5Gy) female NRG-W41 (NOD.Cg-Rag1^{tm1Mom}Kit^{W-41J}Il2rg^{tm1Wjl})) mice aged 8-10 weeks. Mice were housed under conditions of alternating 12 hours light and 12 hours darkness, at 20-24° C., 45-65% humidity, with feeding (LBS biotech: SDS Cat #SDS801730) and water ad libitum.

For treatment with imatinib plus MSDC-0160, eight weeks following transplant, drug treatment was started with both imatinib (LC Laboratories, Cat #1-5508) dosed at 100 mg/kg/day (50 mg per kg body weight BID) oral gavage and MSDC-0160 (30 mg/kg; oral gavage once daily). Treatment was given for 4 weeks.

For treatment with asciminib (ABL001) plus MSDC-0160, twelve weeks following transplant, drug treatment was started with both asciminib (Fisher Scientific Cat #1642956) dosed at 10 mg/kg/day (5 mg/kg BID); oral gavage and MSDC-0160 (Apex Biotechnology: Cat #B3702) dosed at 30 mg/kg; oral gavage once daily. Treatment was given for 4 weeks.

At the respective endpoints, bone marrow cells were collected. This was done by placing inverted cut leg bones into 0.5 mL Eppendorf tubes with holes at bottom. These in turn were placed within 1.5 mL Eppendorf tubes containing PBS, centrifuged (12,000 RCF, 20 seconds). Cells were stained (300 μl/test) with anti-mouse (APC-Cy7 BD Biosciences: Cat #557659, RRID: AB_396774, 1 μl), anti-human CD45 (FITC; BD Biosciences: Cat #555482, RRID: AB_395874, 10 μl), anti-human CD34 (APC; BD Biosciences: Cat #555824, RRID: AB_398614, 2 μl) and anti-human CD38 (PerCP; BioLegend: Cat #303520, RRID: AB_893313, 2 μl) antibodies for flow cytometry analysis as described above.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example+/−10%.

For standard molecular biology techniques, see Sambrook, J., Russel, D. W. Molecular Cloning, A Laboratory Manual. 3 ed. 2001, Cold Spring Harbor, New York: Cold Spring Harbor Laboratory Press.

### REFERENCES

1. Chen, C., et al. Oxidative phosphorylation enhances the leukemogenic capacity and resistance to chemotherapy of B cell acute lymphoblastic leukemia. Science Advances 7, eabd6280 (2021).
2. Škrtić, M., et al. Inhibition of Mitochondrial Translation as a Therapeutic Strategy for Human Acute Myeloid Leukemia. Cancer Cell 20, 674-688 (2011).
3. Zhang, L., et al. Metabolic reprogramming toward oxidative phosphorylation identifies a therapeutic target for mantle cell lymphoma. Science Translational Medicine 11, eaau1167 (2019).
4. Ye, H., et al. Leukemic Stem Cells Evade Chemotherapy by Metabolic Adaptation to an Adipose Tissue Niche. Cell Stem Cell 19, 23-37 (2016).
5. Farge, T., et al. Chemotherapy-Resistant Human Acute Myeloid Leukemia Cells Are Not Enriched for Leukemic Stem Cells but Require Oxidative Metabolism. Cancer Discovery 7, 716-735 (2017).
6. Raffel, S., et al. BCAT1 restricts αKG levels in AML stem cells leading to IDHmut-like DNA hypermethylation. Nature 551, 384-388 (2017).
7. Gallipoli, P., et al. Glutaminolysis is a metabolic dependency in FLT3ITD acute myeloid leukemia unmasked by FLT3 tyrosine kinase inhibition. Blood 131, 1639-1653 (2018).
8. Guièze, R., et al. Mitochondrial Reprogramming Underlies Resistance to BCL-2 Inhibition in Lymphoid Malignancies. Cancer Cell 36, 369-384.e313 (2019).
9. Forte, D., et al. Bone Marrow Mesenchymal Stem Cells Support Acute Myeloid Leukemia Bioenergetics and Enhance Antioxidant Defense and Escape from Chemotherapy. Cell Metabolism 32, 829-843.e829 (2020).
10. Jones, C. L., et al. Nicotinamide Metabolism Mediates Resistance to Venetoclax in Relapsed Acute Myeloid Leukemia Stem Cells. Cell Stem Cell 27, 748-764.e744 (2020).
11. Stevens, B. M., et al. Fatty acid metabolism underlies venetoclax resistance in acute myeloid leukemia stem cells. Nature Cancer 1, 1176-1187 (2020).
12. Kuntz, E. M., et al. Targeting mitochondrial oxidative phosphorylation eradicates therapy-resistant chronic myeloid leukemia stem cells. Nature Medicine 23,1234-1240 (2017).
13. Rowley, J. D. A New Consistent Chromosomal Abnormality in Chronic Myelogenous Leukaemia identified by Quinacrine Fluorescence and Giemsa Staining. Nature 243, 290-293 (1973).
14. Groffen, J., et al. Philadelphia chromosomal breakpoints are clustered within a limited region, bcr, on chromosome 22. Cell 36, 93-99 (1984).
15. Konopka, J. B., Watanabe, S. M. & Witte, O. N. An alteration of the human <em>c-abl</em>protein in K562 leukemia cells unmasks associated tyrosine kinase activity. Cell 37, 1035-1042 (1984).
16. Giralt, S., Kantarjian, H. & Talpaz, M. The natural history of chronic myelogenous leukemia in the interferon era. Semin Hematol 32, 152-158 (1995).
17. Rousselot, P., et al. Imatinib mesylate discontinuation in patients with chronic myelogenous leukemia in complete molecular remission for more than 2 years. Blood 109, 58-60 (2006).
18. Saussele, S., et al. Discontinuation of tyrosine kinase inhibitor therapy in chronic myeloid leukaemia (EURO-SKI): a prespecified interim analysis of a prospective, multicentre, non-randomised, trial. Lancet Oncol 19, 747-757 (2018).
19. Huang, X., Cortes, J. & Kantarjian, H. Estimations of the increasing prevalence and plateau prevalence of chronic myeloid leukemia in the era of tyrosine kinase inhibitor therapy. Cancer 118, 3123-3127 (2012).
20. Copland, M., et al. Dasatinib (BMS-354825) targets an earlier progenitor population than imatinib in primary CML but does not eliminate the quiescent fraction. Blood 107, 4532-4539 (2006).
21. Hamilton, A., et al. Chronic myeloid leukemia stem cells are not dependent on Bcr-Abl kinase activity for their survival. Blood 119, 1501-1510 (2012).
22. Ng, K. P., et al. Physiologic hypoxia promotes maintenance of CML stem cells despite effective BCR-ABL1 inhibition. Blood 123, 3316-3326 (2014).
23. Corbin, A. S., et al. Human chronic myeloid leukemia stem cells are insensitive to imatinib despite inhibition of BCR-ABL activity. J Clin Invest 121, 396-409 (2011).
24. Mitchell, R., et al. Targeting BCR-ABL-Independent TKI Resistance in Chronic Myeloid Leukemia by mTOR and Autophagy Inhibition. J Nat/Cancer Inst 110, 467-478 (2018).
25. Colca, J. R., et al. Clinical proof-of-concept study with MSDC-0160, a prototype mTOT-modulating insulin sensitizer. Clin Pharmacol Ther 93, 352-359 (2013).
26. Shah, R. C., et al. An evaluation of MSDC-0160, a prototype mTOT modulating insulin sensitizer, in patients with mild Alzheimer's disease. Curr Alzheimer Res 11, 564-573 (2014).
27. Divakaruni, A. S., et al. Thiazolidinediones are acute, specific inhibitors of the mitochondrial pyruvate carrier. Proc Nat/Acad Sci USA 110, 5422-5427 (2013).
28. Scott, M. T., et al. Epigenetic Reprogramming Sensitizes CML Stem Cells to Combined EZH2 and Tyrosine Kinase Inhibition. Cancer Discovery 6, 1248-1257 (2016).
29. Abraham, S. A., et al. Dual targeting of p53 and c-MYC selectively eliminates leukaemic stem cells. Nature 534, 341-346 (2016).
30. Houshmand, M., et al. Chronic myeloid leukemia stem cells. Leukemia 33, 1543-1556 (2019).
31. Mi, H., et al. Protocol Update for large-scale genome and gene function analysis with the PANTHER classification system (v.14.0). Nature Protocols 14, 703-721 (2019).
32. Lin, K. H., et al. Systematic Dissection of the Metabolic-Apoptotic Interface in AML Reveals Heme Biosynthesis to Be a Regulator of Drug Sensitivity. Cell Metabolism 29, 1217-1231.e1217 (2019).
33. Giustacchini, A., et al. Single-cell transcriptomics uncovers distinct molecular signatures of stem cells in chronic myeloid leukemia. Nature Medicine 23, 692-702 (2017).
34. Wang, Y.-H., et al. Cell-State-Specific Metabolic Dependency in Hematopoiesis and Leukemogenesis. Cell 158, 1309-1323 (2014).
35. Zhu, Y., et al. Targeting PFKFB3 sensitizes chronic myelogenous leukemia cells to tyrosine kinase inhibitor. Oncogene 37, 2837-2849 (2018).
36. Abraham, A., et al. SIRT1 regulates metabolism and leukemogenic potential in CML stem cells. J Clin Invest 129, 2685-2701 (2019).
37. Vande Voorde, J., et al. Improving the metabolic fidelity of cancer models with a physiological cell culture medium. Sci Adv 5, eaau7314 (2019).
38. Peng, B., et al. Pharmacokinetics and Pharmacodynamics of Imatinib in a Phase I Trial With Chronic Myeloid Leukemia Patients. Journal of Clinical Oncology 22, 935-942 (2004).
39. Chu, S., Holtz, M., Gupta, M. & Bhatia, R. BCR/ABL kinase inhibition by imatinib mesylate enhances MAP kinase activity in chronic myelogenous leukemia CD34+ cells. Blood 103, 3167-3174 (2004).
40. Pellicano, F., et al. hsa-mir183/EGR1-mediated regulation of E2F1 is required for CML stem/progenitor cell survival. Blood 131, 1532-1544 (2018).
41. Hardie, D. G. & Hawley, S. A. AMP-activated protein kinase: the energy charge hypothesis revisited. BioEssays 23, 1112-1119 (2001).
42. Ianniciello, A., et al. ULK1 inhibition promotes oxidative stress-induced differentiation and sensitizes leukemic stem cells to targeted therapy. Science Translational Medicine 13, eabd5016 (2021).
43. Bruntz, R. C., Lane, A. N., Higashi, R. M. & Fan, T. W. M. Exploring cancer metabolism using stable isotope-resolved metabolomics (SIRM). Journal of Biological Chemistry 292, 11601-11609 (2017).
44. Bader, D. A., et al. Mitochondrial pyruvate import is a metabolic vulnerability in androgen receptor-driven prostate cancer. Nature Metabolism 1, 70-85 (2018).
45. Sellers, K., et al. Pyruvate carboxylase is critical for non-small-cell lung cancer proliferation. The Journal of Clinical Investigation 125, 687-698 (2015).
46. Miller, P. H., et al. Analysis of parameters that affect human hematopoietic cell outputs in mutant c-kit-immunodeficient mice. Exp Hematol 48, 41-49 (2017).
47. Hanahan, D. & Weinberg, R. A. Hallmarks of cancer: the next generation. Cell 144, 646-674 (2011).
48. van Gastel, N., et al. Induction of a Timed Metabolic Collapse to Overcome Cancer Chemoresistance. Cell Metab 32, 391-403 e396 (2020).
49. Nachef, M., Ali, A. K., Almutairi, S. M. & Lee, S.-H. Targeting SLC1A5 and SLC3A2/SLC7A5 as a Potential Strategy to Strengthen Anti-Tumor Immunity in the Tumor Microenvironment. Frontiers in Immunology 12(2021).
50. Wise David, R., et al. Myc regulates a transcriptional program that stimulates mitochondrial glutaminolysis and leads to glutamine addiction. Proceedings of the National Academy of Sciences 105, 18782-18787 (2008).
51. Zhao, X., Petrashen, A. P., Sanders, J. A., Peterson, A. L. & Sedivy, J. M. SLC1A5 glutamine transporter is a target of MYC and mediates reduced mTORC1 signaling and increased fatty acid oxidation in long-lived Myc hypomorphic mice. Aging Cell 18, e12947 (2019).
52. Cardaci, S., et al. Pyruvate carboxylation enables growth of SDH-deficient cells by supporting aspartate biosynthesis. Nature Cell Biology 17, 1317-1326 (2015).
53. Cheng, T., et al. Pyruvate carboxylase is required for glutamine-independent growth of tumor cells. Proceedings of the National Academy of Sciences 108, 8674-8679 (2011).
54. Psychogios, N., et al. The Human Serum Metabolome. PLOS ONE 6, e16957 (2011).
55. Jabbour, E. J., Cortes, J. E. & Kantarjian, H. M. Resistance to Tyrosine Kinase Inhibition Therapy for Chronic Myelogenous Leukemia: A Clinical Perspective and Emerging Treatment Options. Clinical Lymphoma Myeloma and Leukemia 13, 515-529 (2013).
56. Pietzke, M. & Vazquez, A. Metabolite AutoPlotter—an application to process and visualise metabolite data in the web browser. Cancer & Metabolism 8, 15 (2020).
57. Pang, Z., et al. MetaboAnalyst 5.0: narrowing the gap between raw spectra and functional insights. Nucleic Acids Research 49, W388-W396 (2021).

1. An MPC inhibitor for use in the treatment of chronic myeloid leukemia (CML), wherein the MPC inhibitor is a compound of Formula (I) or a pharmaceutically acceptable salt thereof: 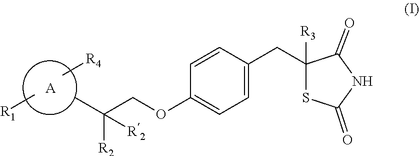
wherein
each of R₁ and R₄ is independently selected from —H, halo, C₁₋₆ alkyl, and C₁₋₆ alkoxy, wherein the C₁₋₆ alkyl and C₁₋₆ alkoxy are optionally substituted with 1-3 of halo;
R′₂ is —H and R₂ is selected from halo, hydroxy, optionally substituted C₁₋₆ alkyl, —OC(O)R^{A}, —O(SO₂)NH₂, —OCH(Rₘ)OC(O)Rₙ, —OCH(Rₘ)OP(O)(ORₙ)₂, —OP(O)(ORₙ)₂, and 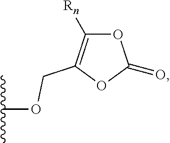
wherein R^{A} is selected from optionally substituted C₁₋₁₂ alkyl, optionally substituted C₆₋₁₂ aryl and optionally substituted C₅₋₁₂ heteroaryl, each Rₘ is independently optionally substituted C₁₋₁₂ alkyl, each Rₙ is independently selected from optionally substituted C₁₋₁₂ alkyl, optionally substituted C₃₋₈ cycloalkyl, and optionally substituted phenyl; or R₂ and R′₂ together form oxo;
R₃ is —H or optionally substituted C₁₋₃ alkyl; and
A is a phenyl, pyridin-2-yl, pyridin-3-yl, or pyridin-4-yl.

2. An MPC inhibitor for use according to claim 1 wherein the MPC inhibitor is for administration in conjunction with a BCR-ABL kinase inhibitor.

3. An MPC inhibitor for use in
(i) inhibiting expansion of the LSC population in CML; and/or
(ii) sensitising LSCs to treatment with a BCR-ABL kinase inhibitor in CML;
wherein the MPC inhibitor is a compound of Formula (I) or a pharmaceutically acceptable salt thereof: 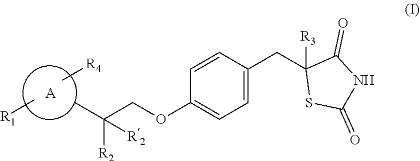
wherein
each of R₁ and R₄ is independently selected from —H, halo, C₁₋₆ alkyl, and C₁₋₆ alkoxy, wherein the C₁₋₆ alkyl and C₁₋₆ alkoxy are optionally substituted with 1-3 of halo;
R′₂ is —H and R₂ is selected from halo, hydroxy, optionally substituted C₁₋₆ alkyl, —OC(O)R^{A}, —O(SO₂)NH₂, —OCH(Rₘ)OC(O)Rₙ, —OCH(Rₘ)OP(O)(ORₙ)₂, —OP(O)(ORₙ)₂, and 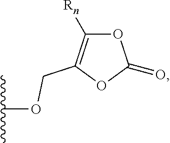
wherein R^{A} is selected from optionally substituted C₁₋₁₂ alkyl, optionally substituted C₆₋₁₂ aryl and optionally substituted C₅₋₁₂ heteroaryl, each Rₘ is independently optionally substituted C₁₋₁₂ alkyl, each Rₙ is independently selected from optionally substituted C₁₋₁₂ alkyl, optionally substituted C₃₋₈ cycloalkyl, and optionally substituted phenyl; or R₂ and R′₂ together form oxo;
R₃ is —H or optionally substituted C₁₋₃ alkyl; and
A is a phenyl, pyridin-2-yl, pyridin-3-yl, or pyridin-4-yl.

4. An MPC inhibitor for use in the treatment of chronic myeloid leukemia (CML), wherein the MPC inhibitor is for administration in combination with a BCR-ABL kinase inhibitor, and wherein the MPC inhibitor is a compound of Formula (I) or a pharmaceutically acceptable salt thereof: 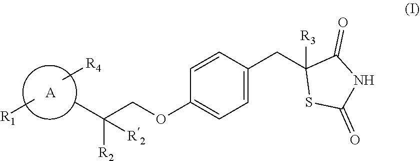
wherein
each of R₁ and R₄ is independently selected from —H, halo, C₁₋₆ alkyl, and C₁₋₆ alkoxy, wherein the C₁₋₆ alkyl and C₁₋₆ alkoxy are optionally substituted with 1-3 of halo;
R′₂ is —H and R₂ is selected from halo, hydroxy, optionally substituted C₁₋₆ alkyl, —OC(O)R^{A}, —O(SO₂)NH₂, —OCH(Rₘ)OC(O)Rₙ, —OCH(Rₘ)OP(O)(ORₙ)₂, —OP(O)(ORₙ)₂, and 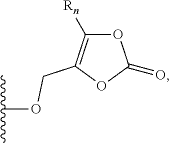
wherein R^{A} is selected from optionally substituted C₁₋₁₂ alkyl, optionally substituted C₆₋₁₂ aryl and optionally substituted C₅₋₁₂ heteroaryl, each Rₘ is independently optionally substituted C₁₋₁₂ alkyl, each Rₙ is independently selected from optionally substituted C₁₋₁₂ alkyl, optionally substituted C₃₋₈ cycloalkyl, and optionally substituted phenyl; or R₂ and R′₂ together form oxo;
R₃ is —H or optionally substituted C₁₋₃ alkyl; and
A is a phenyl, pyridin-2-yl, pyridin-3-yl, or pyridin-4-yl.

5. An MPC inhibitor for use in
(i) inhibiting expansion of the LSC population in CML; and/or
(ii) sensitising LSCs to treatment with a BCR-ABL kinase inhibitor in CML;
wherein the MPC inhibitor is for administration in combination with a BCR-ABL kinase inhibitor, and
wherein the MPC inhibitor is a compound of Formula (I) or a pharmaceutically acceptable salt thereof: 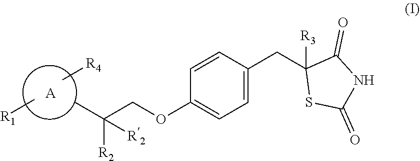
wherein
each of R₁ and R₄ is independently selected from —H, halo, C₁₋₆ alkyl, and C₁₋₆ alkoxy, wherein the C₁₋₆ alkyl and C₁₋₆ alkoxy are optionally substituted with 1-3 of halo;
R′₂ is —H and R₂ is selected from halo, hydroxy, optionally substituted C₁₋₆ alkyl, —OC(O)R^{A}, —O(SO₂)NH₂, —OCH(Rₘ)OC(O)Rₙ, —OCH(Rₘ)OP(O)(ORₙ)₂, —OP(O)(ORₙ)₂, and 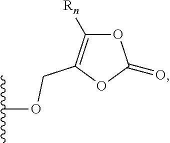
wherein R^{A} is selected from optionally substituted C₁₋₁₂ alkyl, optionally substituted C₆₋₁₂ aryl and optionally substituted C₅₋₁₂ heteroaryl, each Rₘ is independently optionally substituted C₁₋₁₂ alkyl, each Rₙ is independently selected from optionally substituted C₁₋₁₂ alkyl, optionally substituted C₃₋₈ cycloalkyl, and optionally substituted phenyl; or R₂ and R′₂ together form oxo;
R₃ is —H or optionally substituted C₁₋₃ alkyl; and
A is a phenyl, pyridin-2-yl, pyridin-3-yl, or pyridin-4-yl.

6. An MPC inhibitor for use according to any one of claims 2 to 5 wherein the BCR-ABL kinase inhibitor is an active site-binding inhibitor.

7. An MPC inhibitor for use according to claim 6 wherein the BCR-ABL kinase inhibitor is imatinib (ST1571), nilotinib (AMN107), dasatinib (BMS-354825), bosutinib (SKI-606), ponatinib (AP24534) or bafetinib (NS-187).

8. An MPC inhibitor for use according to any one of claims 2 to 5 wherein the BCR-ABL kinase inhibitor is a STAMP inhibitor.

9. An MPC inhibitor for use according to claim 6 wherein the BCR-ABL kinase inhibitor is asciminib (ABL001).

10. An MPC inhibitor for use according to any one of the preceding claims wherein, in Formula (I):
R′₂ is —H and R₂ is selected from hydroxy and —OC(O)R^{A}; or
R₂ and R′₂ together form oxo.

11. An MPC inhibitor for use according to claim 10 wherein R₂ and R′₂ together form oxo.

12. An MPC inhibitor for use according to any one of the preceding claims wherein:
R₁ is selected from C₁₋₆ alkyl optionally substituted with 1-3 of halo and C₁₋₆ alkoxy optionally substituted with 1-3 of halo; and
R₄ is —H.

13. An MPC inhibitor for use according to any one of the preceding claims wherein R₃ is —H.

14. An MPC inhibitor for use according to any one of claims 1 to 9 wherein the MPC inhibitor is a compound of formula (IIa′) or (IIb′), or a pharmaceutically acceptable salt thereof: 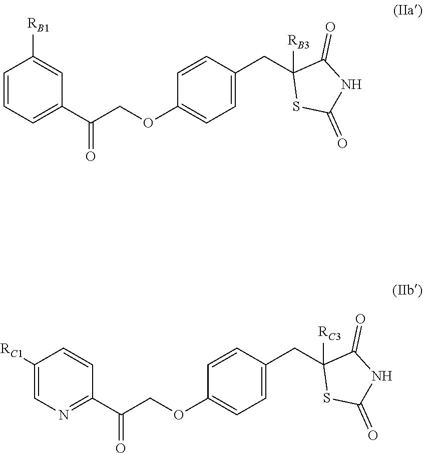
wherein
R_{B1} and R_{C1} are independently selected from —H, halo, C₁₋₆ alkyl, and C₁₋₆ alkoxy, wherein the C₁₋₆ alkyl and C₁₋₆ alkoxy are optionally substituted with 1-3 of halo; and
R_{B3} and R_{C3} are independently selected from —H and optionally substituted C₁₋₃ alkyl.

15. An MPC inhibitor for use according to any one of the preceding claims wherein the MPC inhibitor is selected from Compound A and Compound B:

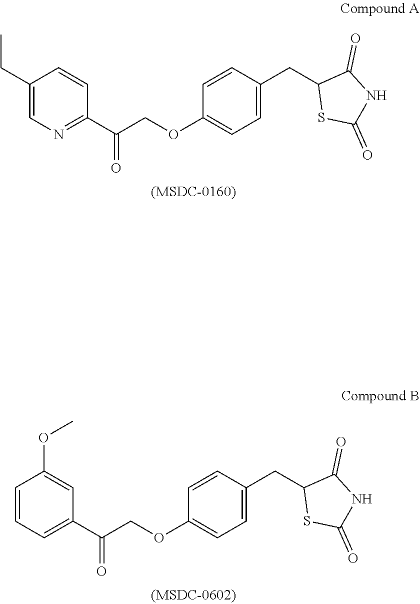 [SMILES: CCc1ccc(C(=O)COc2ccc(CC3SC(=O)NC3=O)cc2)nc1 ; COc1cccc(C(=O)COc2ccc(CC3SC(=O)NC3=O)cc2)c1]

16. A combination therapy comprising
(a) an MPC inhibitor; and
(b) a BCR-ABL kinase inhibitor;
for treatment of CML;
wherein the MPC inhibitor is a compound of Formula (I) or a pharmaceutically acceptable salt thereof: 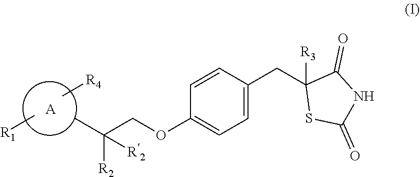
wherein
each of R₁ and R₄ is independently selected from —H, halo, C₁₋₆ alkyl, and C₁₋₆ alkoxy, wherein the C₁₋₆ alkyl and C₁₋₆ alkoxy are optionally substituted with 1-3 of halo;
R′₂ is —H and R₂ is selected from halo, hydroxy, optionally substituted C₁₋₆ alkyl, —OC(O)R^{A}, —O(SO₂)NH₂, —OCH(Rₘ)OC(O)Rₙ, —OCH(Rₘ)OP(O)(ORₙ)₂, —OP(O)(ORₙ)₂, and 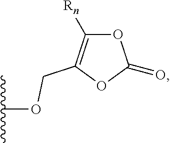
wherein R^{A} is selected from optionally substituted C₁₋₁₂ alkyl, optionally substituted C₆₋₁₂ aryl and optionally substituted C₅₋₁₂ heteroaryl, each Rₘ is independently optionally substituted C₁₋₁₂ alkyl, each Rₙ is independently selected from optionally substituted C₁₋₁₂ alkyl, optionally substituted C₃₋₈ cycloalkyl, and optionally substituted phenyl; or R₂ and R′₂ together form oxo;
R₃ is —H or optionally substituted C₁₋₃ alkyl; and
A is a phenyl, pyridin-2-yl, pyridin-3-yl, or pyridin-4-yl.

17. A combination therapy according to claim 4 wherein the MPC inhibitor is administered for
(i) inhibiting expansion of the LSC population; and/or
(ii) sensitising LSCs to treatment with a BCR-ABL kinase inhibitor.

18. A combination therapy according to claim 16 or claim 17 wherein the BCR-ABL kinase inhibitor is an active site-binding inhibitor.

19. A combination therapy according to claim 18 wherein the BCR-ABL kinase inhibitor is imatinib (ST1571), nilotinib (AMN107), dasatinib (BMS-354825), bosutinib (SKI-606), ponatinib (AP24534) or bafetinib (NS-187).

20. A combination therapy according to claim 16 or claim 17 wherein the BCR-ABL kinase inhibitor is a STAMP inhibitor.

21. A combination therapy according to claim 20 wherein the BCR-ABL kinase inhibitor is asciminib (ABL001).

22. A combination therapy according to any one of claim 16 to 21 wherein, in Formula (I):
R′₂ is —H and R₂ is selected from hydroxy and —OC(O)R^{A}; or
R₂ and R′₂ together form oxo.

23. A combination therapy according to claim 22 wherein R₂ and R′₂ together form oxo.

24. A combination therapy according to any one of claims 16 to 23 wherein:
R₁ is selected from C₁₋₆ alkyl optionally substituted with 1-3 of halo and C₁₋₆ alkoxy optionally substituted with 1-3 of halo; and
R₄ is —H.

25. A combination therapy according to any one of claims 16 to 24 wherein R₃ is —H.

26. A combination therapy according to any one of claims 16 to 21 wherein the MPC inhibitor is a compound of formula (IIa′) or (IIb′), or a pharmaceutically acceptable salt thereof: 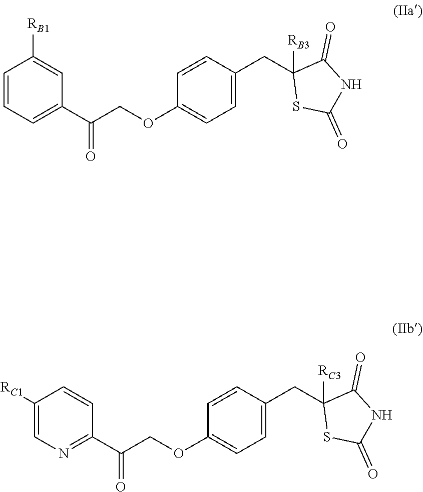
wherein
R_{B1} and R_{C1} are independently selected from —H, halo, C₁₋₆ alkyl, and C₁₋₆ alkoxy, wherein the C₁₋₆ alkyl and C₁₋₆ alkoxy are optionally substituted with 1-3 of halo; and
R_{B3} and R_{C3} are independently selected from —H and optionally substituted C₁₋₃ alkyl.

27. A combination therapy according to any one of claims 16 to 26 wherein the MPC inhibitor is selected from Compound A and Compound B:

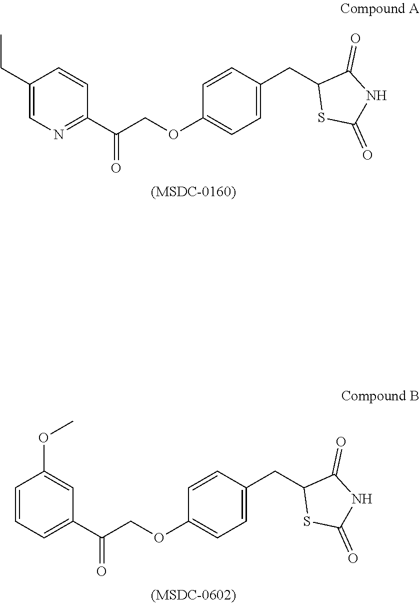 [SMILES: CCc1ccc(C(=O)COc2ccc(CC3SC(=O)NC3=O)cc2)nc1 ; COc1cccc(C(=O)COc2ccc(CC3SC(=O)NC3=O)cc2)c1]

28. A BCR-ABL kinase inhibitor for use in the treatment of CML, wherein the BCR-ABL kinase inhibitor is for administration in combination with an MPC inhibitor of Formula (I) or a pharmaceutically acceptable salt thereof: 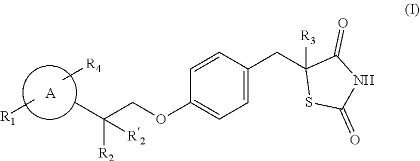
wherein
each of R₁ and R₄ is independently selected from —H, halo, C₁₋₆ alkyl, and C₁₋₆ alkoxy, wherein the C₁₋₆ alkyl and C₁₋₆ alkoxy are optionally substituted with 1-3 of halo;
R′₂ is —H and R₂ is selected from halo, hydroxy, optionally substituted C₁₋₆ alkyl, —OC(O)R^{A}—O(SO₂)NH₂, —OCH(Rₘ)OC(O)Rₙ, —OCH(Rₘ)OP(O)(ORₙ)₂, —OP(O)(ORₙ)₂, and 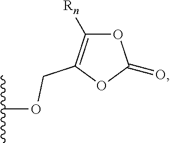
wherein R^{A} is selected from optionally substituted C₁₋₁₂ alkyl, optionally substituted C₆₋₁₂ aryl and optionally substituted C₅₋₁₂ heteroaryl, each Rₘ is independently optionally substituted C₁₋₁₂ alkyl, each Rₙ is independently selected from optionally substituted C₁₋₁₂ alkyl, optionally substituted C₃₋₈ cycloalkyl, and optionally substituted phenyl; or R₂ and R′₂ together form oxo;
R₃ is —H or optionally substituted C₁₋₃ alkyl; and
A is a phenyl, pyridin-2-yl, pyridin-3-yl, or pyridin-4-yl.

29. A BCR-ABL kinase inhibitor for use according to claim 28 wherein the MPC inhibitor is administered for
(i) inhibiting expansion of the LSC population; and/or
(ii) sensitising LSCs to treatment with a BCR-ABL kinase inhibitor.
